(19) United States
(12) Patent Application Publication
(10) Pub. No.: US 2019/0328857 A1
(43) Pub. Date: Oct. 31, 2019

(54) CALR AND JAK2 VACCINE COMPOSITIONS

(71) Applicant: IO Biotech ApS, Copenhagen N (DK)

(72) Inventors: Mads Hald ANDERSEN, Nærum (DK); Morten Orebo HOLMSTRÖM, Roskilde (DK); Hans HASSELBALCH, Søborg (DK)

(21) Appl. No.: 16/308,164

(22) PCT Filed: Jun. 9, 2017
(86) PCT No.: PCT/DK2017/050190

§ 371 Date: Dec. 7, 2018

(30) Foreign Application Priority Data
Jun. 10, 2016 (DK) PA 2016 70417

(51) Int. Cl. A61K 39/00; A61P 35/00; A61K 39/39

(52) U.S. Cl. CPC A61K 39/001162; A61P 35/00; A61K 45/06; A61K 2039/5154; A61K 2039/545; A61K 39/39

(57) ABSTRACT

The present disclosure relates to CALR and JAK2 as novel T cell targets in prophylaxis and treatment of a myeloproliferative disorder.

### FIELD OF INVENTION

The present invention relates to the field of prophylaxis and therapy of disorders, such as myeloproliferative disorders, in particular myeloproliferative neoplasms (MPNs). In particular there is provided peptide fragments from the mutated JAK2- and CALR genes that are capable of eliciting immune responses against these hematological cancers. Specifically, the invention relates to the use of peptides derived from the JAK2- and CALR-mutations and thereof from CALR-specific or JAK2-specific T-cells for treatment of MPNs. The invention thus relates to vaccines, which optionally may be used in combination with other immunotherapies. The invention may also be used for the adoptive transfer of T-cells specific for the JAK2 mutation and CALR mutations or be used to induce in vivo immunity to the JAK2 and CALR mutated peptide by vaccination as a treatment of MPNs. It is an aspect of the invention that the vaccine strategies herein provided may be used in combination with cancer chemotherapy. A further aspect relates to prophylaxis and therapy of myeloproliferative disorders such as essential thrombocythaemia, primary myelofibrosis or polycythaemia vera.

The use of immunogenically active peptide fragments derived from CALR and JAK2 mutants for treatment, diagnosis and prognosis of proliferative disorders is also provided.

### BACKGROUND OF INVENTION

More than 50% of patients with MPNs harbor the JAK2V617F mutation. In addition, mutations in exon 9 of the calreticulin (CALR) gene are found in approximately 60% of patients with JAK2 wild type essential thrombocytemia (ET) or primary myelofibrosis (PMF).

CALR exon 9 mutations result in a 1-bp frameshift mutation, which alters the C-terminus of the CALR protein (Klampfl et al., 2013; Nangalia et al, 2013). To date, more than 50 mutations have been described, and they all share a 36 amino acid-long consensus sequence in the C-terminus: The two most prominent CALR exon 9 mutations are found in 80% of all patients. This novel C-terminus is a potential tumor associated antigen. Such antigens are believed to be important for the immune system to obtain tumor control, as the immune system allegedly targets the tumor associated antigens (Schumacher et al., 2015).

The CALR exon 9 mutations and the JAK2V617F mutation are found exclusively in myeloid malignancies. They are therefore cancer-specific antigens. Exon 9 CALR mutants and JAK2V617F are consequently an attractive target for cancer immune therapy.

There is a need for methods of treatment and prophylaxis of myeloproliferative disorders such as MPNs.

### SUMMARY OF INVENTION

The invention is as defined in the claims.

The problem of providing such methods of treatment and prophylaxis of proliferative disorders, such as myeloproliferative disorders is solved by the present invention, which provides CALR and JAK2 as novel T cell targets. The inventors have surprisingly found that peptides derived from mutant CALR and JAK2 as defined below, are immunogenic and can elicit an immune response against cells expressing mutant CALR and/or mutant JAK2. Such peptides are thus useful in methods of treatment and prophylaxis of myeloproliferative disorders such as MPNs. The peptides disclosed herein can elicit a T-cell response and can be used to manufacture T cell vaccines useful in methods of treatment and prophylaxis of myeloproliferative disorders.

Thus, the present invention provides materials and methods for treatment of MPNs, by inducing an immune response targeting cells expressing mutant CALR or mutant JAK2 directly and thereby killing these cells.

This is done by enabling T cells to recognize the cells expressing mutant CALR or mutant JAK2. Interestingly, the present invention discloses that cytotoxic immune responses against cells expressing mutant JAK2 or CALR can be raised in healthy subjects, and that T-cell responses against the mutant CALR are identified in healthy donors. This provides a novel mechanism for treating and/or preventing myeloproliferative disorders such as MPNs.

The inventors herein disclose, that T cells are capable of killing antigen-presenting cells (APCs) presenting mutant JAK2 and that T cells are massively activated when exposed to CALR mutant peptide.

Thus, the invention exploits expression of the mutant CALR and/or JAK2 in MPN cells by stimulating the immune system to target cells expressing mutated JAK2 and/or CALR.

It is an aspect of the invention to provide vaccine compositions which surprisingly can generate an immune response against mutant JAK2 and/or mutant CALR.

### DESCRIPTION OF DRAWINGS

FIG. 1: (A) Example of triplicate ELISPOT with a response against CALR Long1 from patient P37. (B) Bar plot depicting the difference in the mean amount of spots between cells stimulated with CALR Long1 and control wells. (C) Example of triplicate ELISPOT with response against CALR Long2 from patient C39. (D) Bar plot depicting the difference in the mean amount of spots between cells stimulated with CALR Long2 and control wells. * denotes p≤105.

FIG. 2: (A) Intracellular cytokine staining on PBMCs from patient C42 that had been pulsed for a week with CALR Long1 peptide shows a strong CD8 T-cell and a more modest CD4 T-cell response against the peptide. (B) Intracellular cytokine staining on T-cells generated from PBMCs from patient C42 that were stimulated three times with autologous dendritic cells, which had been pulsed with CALR Long1 peptide. The experiment demonstrates an increased reactivity of the CD4 T-cells and a slightly lower reactivity of the CD8 T-cells against CALR Long1 peptide.

FIG. 3: Responses against CALR Long1 and CALR Long2 in TNF-α ELISPOT. (A) Example of triplicate wells with response against CALR Long2. (B) Bar chart displaying difference in spots between cells that were stimulated with CALR Long1, and control cells. (C) Bar chart displaying difference in spots between cells that were stimulated with CALR Long2, and control cells. * denotes p≤0.05.

FIG. 4: Responses against nonamer peptides from the mutated CALR sequence in healthy donors analyzed by IFN-γ ELISPOT. Nonameric peptides in both the CALR Long1 and CALR Long2 peptides elicit strong spontaneous immune responses in healthy donors. * denotes p≤0.05.

FIG. 5: Intracellular cytokine stain on PBMCs stimulated with CALR Long1 peptide. The two healthy donors display a CD4 T-cell response upon stimulation with CALR Long1 peptide.

FIG. 6: T-cell response requires expression of JAK2 mutant polypeptide.

FIG. 7: Specificity of the CD4+ T-cells in the CALRLong1-bulk culture to epitopes in the mutant CALR C-terminus. Cells stimulated with CALRLong1 (top, left), stimulation with CALRLong3 (middle, left), stimulation with CALRLong2 (bottom, left), stimulation with CALRLong4 (top, right), stimulation with CALRLong5 (middle, right) and a scrambled peptide.

FIG. 8: Establishment of a CD4⁺ T-cell culture specific for CALRLong1 peptide (RRMMRTKMRMRRMRRTRRKMRRKMSPARP). A. The top row shows specificity of the bulk culture after three stimulations with dendritic cells. The middle row shows specificity after IFN-γ enrichment of the bulk culture. The bottom row displays specificity of the CD4⁺ CALRLong1-specific T-cells. B. Phenotypic analysis of the CD4⁺ CALRLong1-specific T-cells.

FIG. 9: The CD4⁺ CALRLong1 specific T-cells recognize autologous CALRmut cells. A. The specific T-cells were stimulated with autologous CALRmut CD14⁺ monocytes at an effector:target ratio of 1:1. B. The specific T-cells were stimulated with autologous CALRmut CD14⁺ monocytes at an effector:target ratio of 3:1. C. Stimulation of the specific T-cells with autologous EBV-transformed B-cells at an effector:target ratio of 1:1. D. Purity analysis of the CD14⁺ enrichment.

FIG. 10: Recognition of autologous CALRmut target cells is enhanced by target cell stimulation with IFN-γ and decreases when target cells are transfected with CALR siRNA. A. CALRLong1-specific T-cells were stimulated with autologous CD14⁺ monocytes or autologous EBV transformed B-cells (BCL). To enhance antigen presentation, target cells were stimulated with IFN-γ 300 U/ml culture for 24 h before assaying. The effector:target ratio was 1:1 in all experiments. B. Autologous myeloid cells were either transfected with negative control RNA or transfected with CALR siRNA and then used as target cells in an intracellular cytokine staining with the CD4⁺ CALRLong1-specific T-cells as effector cells. C. For transfection control, autologous myeloid cells were transfected with FITC-conjugated siRNA (left) using the same electroporation parameters as the target cells in B. Cells transfected with negative control (right) were used to set gates.

FIG. 11: The CALRLong1-specific response is HLA class II-restricted with HLA-DR as the restriction element. A. The CD4⁺ CALRLong1 specific T-cells were stimulated with autologous CD14⁺ monocytes at an effector:target ratio of 1:1. Monocytes were either left untreated (A, top) or had been treated for 30 min with an HLA class II-blocking monoclonal antibody (T039) (A, bottom). B. The CD4⁺ CALRLong1 specific T-cells were stimulated with CALRLong1 peptide and then either left untreated (B, top), treated with an HLA-DQ-specific antibody for 15 min (B, middle) or an HLA-DR-specific antibody for 15 min (B, bottom).

FIG. 12: The CALRLong1-specific T-cells recognize autologous CD34⁺ cells from bone marrow and blood. A. The CALRLong1-specific T-cells were stimulated with autologous CD34⁺ monocytes isolated from a freshly drawn bone marrow aspiration at an effector:target ratio of 3:1 (top), or with a scrambled negative control peptide. Because of the limited amount of target cells, the experiment was performed in duplicates. B. Purity analysis of the CD34⁺ enriched cells from the bone marrow aspiration. C. Stimulation of the CALRLong1-specific T-cells with autologous CD34⁺ cells isolated from cryopreserved PBMC (top) and scrambled negative control (bottom). Both experiments were run with an effector:target ratio of 5:1. The isolated CD34⁺ cells were rested in X-VIVO with 5% human serum for 48 h before assaying. Due to lack of target cells, the experiment was run in one well. D. Purity analysis of the CD34⁺-enriched cells from the cryopreserved PBMC.

FIG. 13: The CD4⁺ CALRLong1-specific T-cells are cytotoxic to autologous cells pulsed with CALRLong1 peptide. A. The CALRLong1-specific T-cells were stimulated with autologous EBV-transformed B-cells (BCL) that had been pulsed with CALRLong1 peptide (top) or a scrambled peptide (bottom). B. Killing curve from a standard Cr⁵¹-cytotoxicity assay, where CALRLong1 specific T-cells were incubated with either BCL pulsed with CALRLong1 or BCL pulsed with a scrambled peptide. C. CD4⁺ CALRLong1-specific T-cells were stimulated with BCL pulsed with either CALRLong1 (top) or scrambled peptide (bottom) and then stained with a PE-conjugated CD107a antibody. D. Cells from the CALRLong1 bulk culture were stimulated with CALRLong1 (top) or scrambled peptide (bottom) and then stained with a PE-conjugated CD107a antibody.

FIG. 14: Spontaneous immune responses against peptide CALRLong1 in healthy donors (example 3). Of 21 analyzed patients, 17 (81%) displayed a significant response against the peptide.

FIG. 15: Spontaneous immune responses against peptide CALRLong2 in healthy donors (example 3). Of 23 analyzed patients, 18 (78%) displayed a significant response against the peptide.

FIG. 16: Spontaneous immune responses against peptide CALRLong4 in healthy donors (example 3). Of 9 analyzed patients, 9 (100%) displayed a significant response against the peptide.

FIG. 17: Spontaneous immune responses against peptide CALRLong5 in healthy donors (example 3). Of 7 analyzed patients, 7 (100%) displayed a significant response against the peptide.

FIG. 18: Spontaneous immune responses in healthy donors against peptide CALRLong1 and CALRLong2 analyzed by TNF-α ELISPOT. A. Ten healthy controls were analyzed for TNF-α response against CALRLong1 and 3 had a significant response. All of these 3 responders harbored an IFN-γ response against CALRLong1 as well. Ten healthy controls were analyzed for TNF-α response against CALRLong2. Five harbored a significant response, and all of these 5 individuals had an IFN-γ response against CALRLong2 as well. B. Ten healthy controls were analyzed for TNF-α response against CALRLong2. Five harboured a significant response, and all of these 5 individuals had an IFN-γ response against CALRLong2 as well.

FIG. 19: Ten healthy controls were screened for spontaneous immune responses using in vitro IFN-γ ELISPOT against nonamer peptides in the mutant CALR C-terminus. Peptide B1 is the first nonamer peptide in the mutant C-terminus, B2 is the second and so forth. We identified immune responses against nonamer epitopes in all parts of the mutant CALR C-terminus, however the majority were identified in the first half of the sequence with peptide B11 as the most immunogenic nonamer epitope.

FIG. 20: Intracellular cytokine staining was used to phenotype the IFN-γ secreting cells identified in the above mentioned ELISPOT analysis of the CALR-mutant nonamer library. The cells that secreted cytokines upon stimulation with CALR-mutant peptides were CD4+ T-cells (A) or mostly CD4+ T-cells (B). However one CD8+ T-cell response in donor BC342 was identified upon stimulation with peptide B11. Donor BC348 displayed a very strong CD4+ response upon stimulation with peptide C2 (B). In total five donors were analyzed, and we identified a response against at least one peptide in four of the donors.

FIG. 21: Specificity of JAK201-specific T cells. (a) ELISPOT assay demonstrating release of IFN-γ (left) and TNF-α (right) from JAK201-specific T cells upon stimulation with JAK201 peptide compared with HIV controls. (b) ELISPOT assay demonstrating release of IFN-γ (left) and TNF-α (right) from JAK201-specific T cells upon stimulation with JAK201 peptide compared with JAK201 wt peptide. The distribution-free resampling method described by Moodie et al. was used for statistical analysis of triplicates. P≤0.05 was considered statistically significant. (c) Standard Cr⁵¹ cytoxicity assay with titration of the concentration of JAK201 and JAK201 wt peptides. (d) Standard Cr⁵¹ cytoxicity assay with JAK201-specific T cells used as effector cells and either K562 cells transfected with HLA-A2 or HLA-A3 pulsed with JAK201 peptide or HLA-A2-transfected K562 cells without any peptide used as target cells.

FIG. 22: Cytolytic capacity of JAK201-specific T cells. (a) IFN-γ ELISPOT assay examining the reactivity of the JAK201-specific T cells towards the JAK2V617F-mutated cancer cells UKE-1 and JAK2 wt, HLA-A2-transfected K562 cells, the UKE-1 cells pretreated with IFN-γ for 2 days, and the HLA-A2-transfected K562 cells pretreated with IFN-γ for 2 days. Experiments were performed in triplicates or duplicates. The distribution-free resampling method described by Moodie et al. was used for statistical analysis of triplicates. P≤0.05 was considered statistically significant. (b) Standard Cr⁵¹ cytoxicity assay with JAK201-specific T cells used as effector cells and the HLA-A2-positive acute myeloid leukemia cancer cell line UKE-1 harboring the JAK2V617F mutation without or with pretreatment with IFN-γ for 2 days used as target cells. UKE-1 cells are homozygous for the JAK2V617F mutation. T2 cells were in addition used as target cells as controls. (c) Standard Cr⁵¹ cytoxicity assay with JAK201-specific T cells used as effector cells and UKE-1 cells either transfected with JAK2V617F siRNA or mock-transfected used as target cells. (d) ELISPOT assay examining the reactivity of the JAK201-specific T cells towards the HLA-A2-positive, JAK2 wt acute myeloid leukemia cancer cell line THP-1. Cells were either transfected with JAK2V617F encoding mRNA or with mRNA encoding NGFR for control. The T cells secrete more IFN-γ (left), TNF-α (middle) and Granzyme B (right) upon stimulation with JAK2V617F-mutated THP-1 cells compared with controls. The distribution-free resampling method described by Moodie et al. was used for statistical analysis of triplicates. P≤0.05 was considered statistically significant.

### DEFINITIONS

Adjuvant: Any substance whose admixture with an administered immunogenically active peptide/antigen/nucleic acid construct increases or otherwise modifies the immune response to said peptide/antigen.

Antigen: Any substance that can bind to a clonally distributed immune receptor (T-cell or B-cell receptor). Usually a peptide, polypeptide or a multimeric polypeptide. Antigens are preferably capable of eliciting an immune response.

APC: Antigen-presenting cell. An APC is a cell that displays antigen complexed with MHC on its surface. T-cells may recognize this complex using their T-cell receptor (TCR). APCs fall into two categories: professional (of which there are three types: Dendritic cells, macrophages and B-cells) or non-professional (does not constitutively express the Major histocompatibility complex proteins required for interaction with naive T cells; these are expressed only upon stimulation of the non-professional APC by certain cytokines such as IFN-γ).

Boost: To boost by a booster shot or dose is to administer an additional dose of an immunizing agent, such as a vaccine, administered at a time after the initial dose to sustain the immune response elicited by the previous dose of the same agent.

Carrier: Entity or compound to which antigens are coupled to aid in the induction of an immune response.

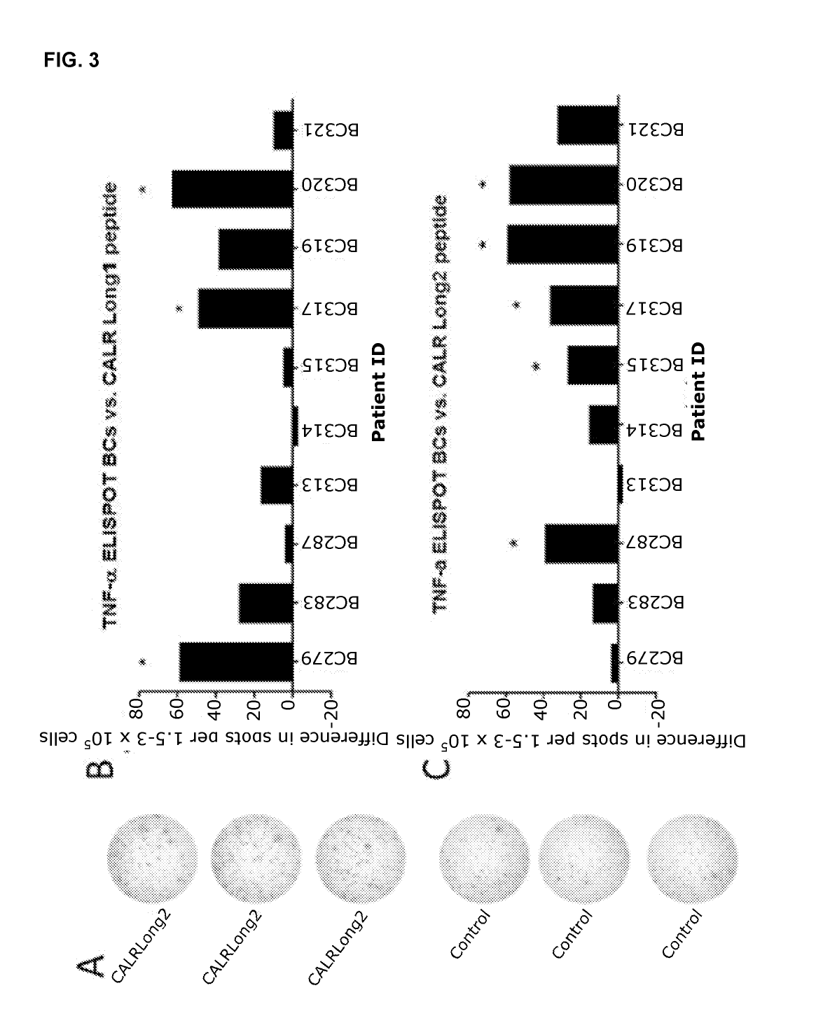

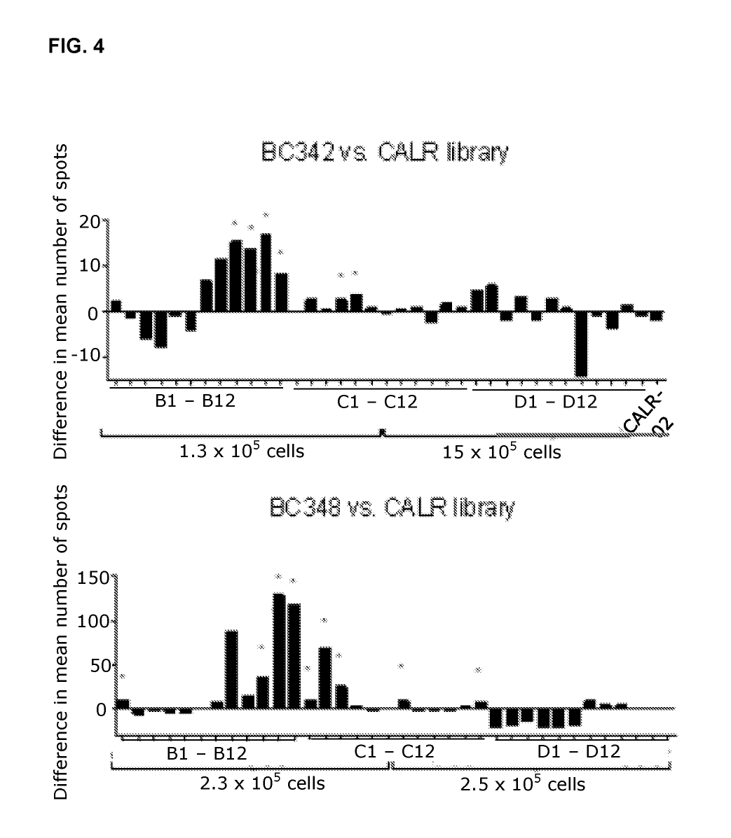

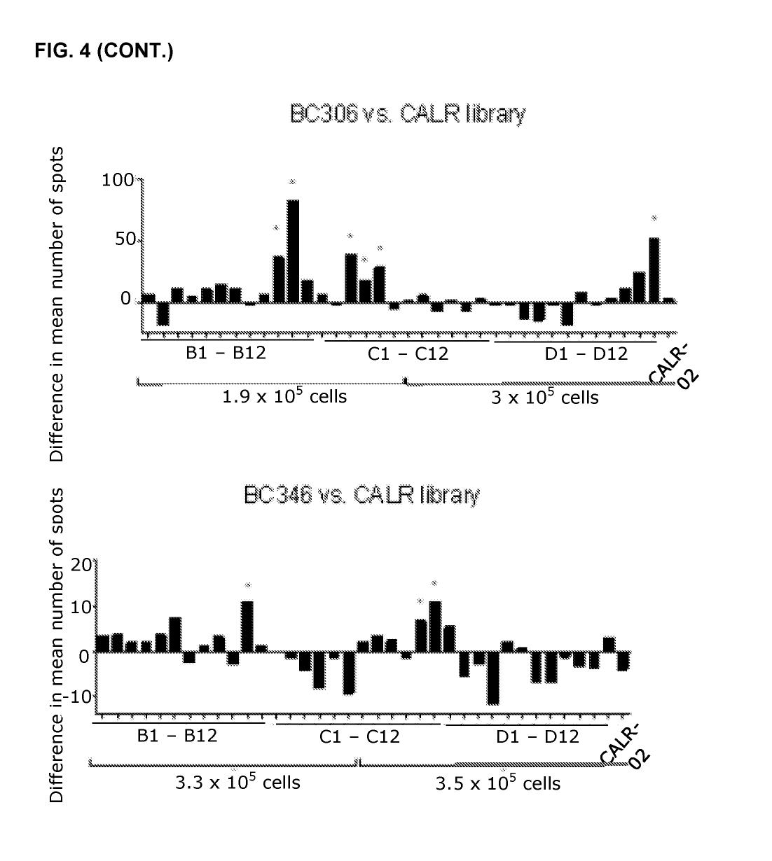

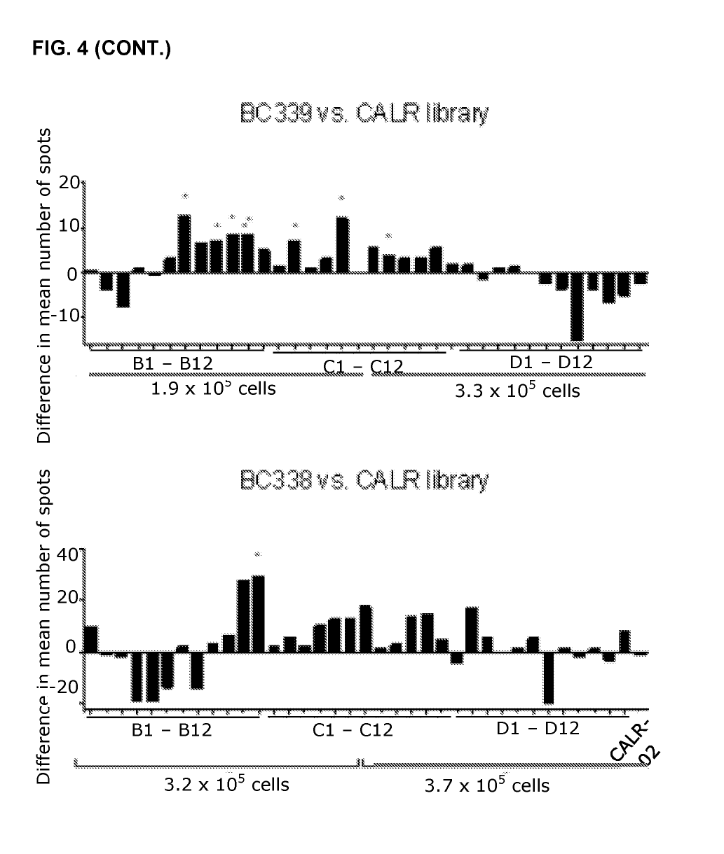

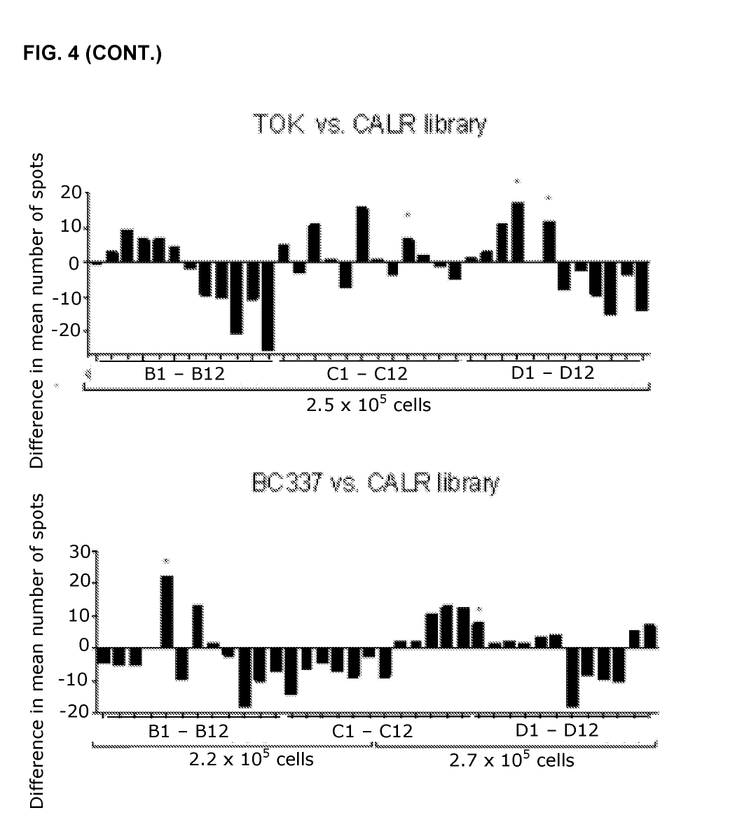

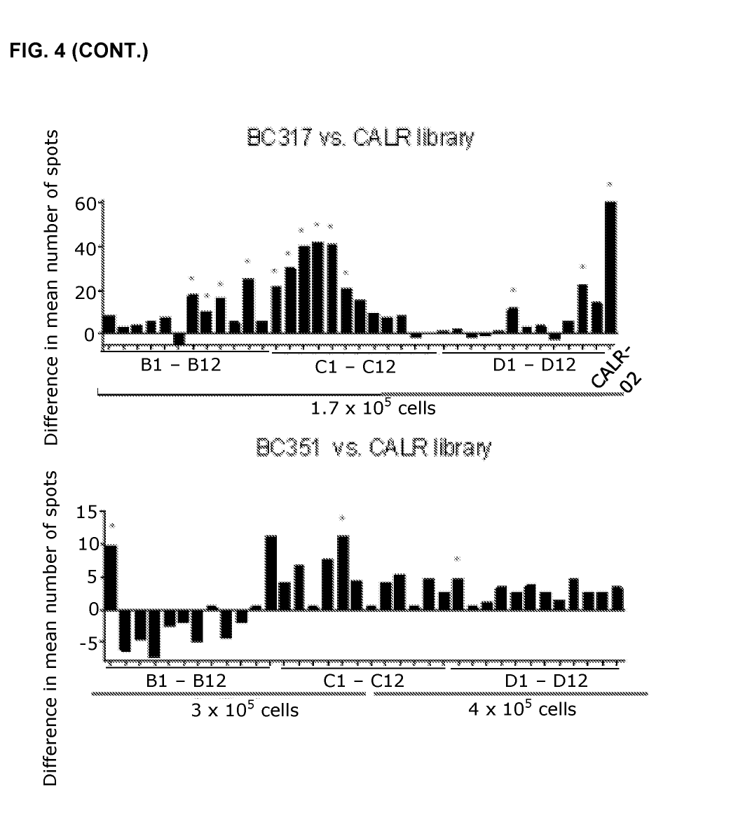

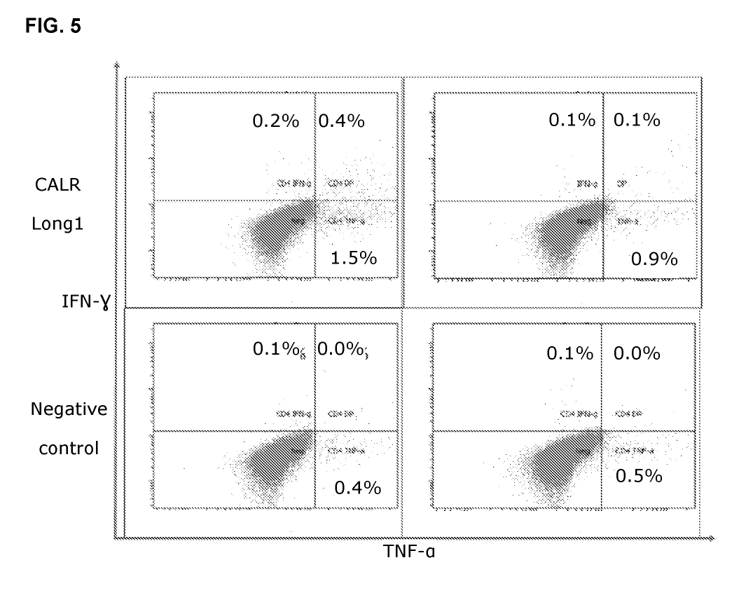

CALR: the CALR gene encodes Calreticulin in humans. Calreticulin is also known as calregulin, CRP55, CaBP3, calsequestrin-like protein, and endoplasmic reticulum resident protein 60 (ERp60). The term “exon 9 mutant CALR” refers throughout this disclosure to a mutant calregulin comprising at least one non-silent mutation in the exon 9 of CALR. For example, an exon 9 mutant CALR may comprise the amino acid sequence SEQ ID NO: 16. Examples of exon 9 mutants are L367fs*46 (full length set forth in SEQ ID NO: 10), E370fs*43, E370fs*48, L367fs*48, L367fs*44, K368fs*51, L367fs*52, R366fs*53, E371fs*49, K368fs*43, E370fs*37, D373fs*47, K374fs*53, E371fs*49, K385fs*47, K385fs*47, R376fs*55, K385fs*47, E381fs*48 (Nangalia et al., 2013; the sequences of the above mutations are listed in FIG. 3, panel A, p. 2400 of Nangalia et al.). Typically, said exon 9 mutant CALR will comprise N-terminal sequences, which are shared with the wild type calreticulin protein. Thus, the exon 9 mutant CALR may comprise amino acid 1 to 360 of SEQ ID NO:10 in addition to the sequence of SEQ ID NO:1. Thus, the exon 9 mutant CALR may be a polypeptide as set forth in SEQ ID NO: 10. In other words, the exon 9 mutant CALR may comprise the amino acid sequence as set forth in SEQ ID NO: 16.

CALR_{xx-yy}: As used herein this nomenclature refers to a polypeptide fragment of CALR consisting of amino acids xx-yy of the full length sequence referred to, i.e. SEQ ID NO: 9.

Exon 9 mutant CALR_{xx-yy}: As used herein this nomenclature refers to a polypeptide fragment of CALR consisting of amino acids xx-yy of the sequence SEQ ID NO: 10.

Chimeric protein: A genetically engineered protein that is encoded by a nucleotide sequence made by a splicing together of two or more complete or partial genes or a series of (non)random nucleic acids.

Clinical condition: A condition that requires medical attention, herein especially conditions associated with the expression of CALR or JAK2, in particular mutant forms of CALR or JAK2. Examples of such conditions include: proliferative disorders, such as myeloproliferative disorders, e.g. cancers.

CTL: Cytotoxic T lymphocyte. A subgroup of T-cells expressing CD8 along with the T-cell receptor and therefore able to respond to antigens presented by class I molecules.

Delivery vehicle: An entity whereby a nucleotide sequence or polypeptide or both can be transported from at least one media to another.

DC: Dendritic cell. (DCs) are immune cells and form part of the mammalian immune system. Their main function is to process antigen material and present it on the surface to other cells of the immune system, thus functioning as antigen-presenting cells (APCs).

Fragment: is used to indicate a non-full length part of a nucleic acid or polypeptide. Thus, a fragment is itself also a nucleic acid or polypeptide, respectively.

Functional homologue: A functional homologue may be any polypeptide that exhibits at least some sequence identity with a reference polypeptide and has retained at least one aspect of the original functionality. Herein a functional homologue of CALR or JAK2 or an immunogenically active peptide fragment thereof is a polypeptide sharing at least some sequence identity with CALR or JAK2 or a fragment thereof and which has the capability to induce an immune response to cells expressing CALR or JAK2. A functional homologue of an exon 9 mutant CALR or JAK2V617F mutant or an immunogenically active peptide fragment thereof is a polypeptide sharing at least some sequence identity with exon 9 mutant CALR of SEQ ID NO: 10 or JAK2V617F of SEQ ID NO: 6 or a fragment thereof and which has the capability to induce an immune response to cells expressing exon 9 mutant CALR or JAK2V617F. Typically, a functional homologue of JAK2V617F comprises at least amino acid 617 of SEQ ID NO: 6. A functional homologue of exon 9 mutant CALR typically comprises at least part of SEQ ID NO: 16.

Immunogenically active peptide: Peptide capable of eliciting an immune response, preferably a T-cell response, in at least one individual after administration to said individual. Peptides may be identified as immunogenically active using any suitable method, including in vitro. For example, a peptide may be identified as immunogenically active if it has at least one of the following characteristics:
(i) It is capable of eliciting INF-γ-producing cells in a PBL population of at least one cancer patient at a frequency of at least 1 per 10⁴ PBLs as determined by an ELISPOT assay, and/or
(ii) It is capable of in situ detection in a sample of tumor tissue of CTLs that are reactive with the epitope peptide; and/or
(iii) It is capable of inducing the growth of specific T-cells in vitro.

Methods suitable for determining whether a peptide is immunogenically active are also provided in the “Examples” section below.

Individual: Generally any species or subspecies of bird, mammal, fish, amphibian, or reptile, preferably a mammal, most preferably a human being.

Infection: Herein the term “infection” relates to any kind of clinical condition involving an invasion of the host organism by disease-causing agents. In particular, infection refers to a clinical condition involving invasion of an individual by a pathogen.

Isolated: used in connection with nucleic acids, polypeptides, and antibodies disclosed herein ‘isolated’ refers to these having been identified and separated and/or recovered from a component of their natural, typically cellular, environment. Nucleic acids, polypeptides, and antibodies of the invention are preferably isolated, and vaccines and other compositions of the invention preferably comprise isolated nucleic acids, polypeptides or isolated antibodies.

JAK2: the JAK2 gene codes for the non-receptor tyrosine kinase Janus kinase 2, which is a member of the Janus kinase family and has been implicated in signaling by members of the type II cytokine receptor family (e.g. interferon receptors), the GM-CSF receptor family (IL-3R, IL-5R and GM-CSF-R), the gp130 receptor family (e.g., IL-6R), and the single chain receptors (e.g. Epo-R, Tpo-R, GH-R, PRL-R). JAK2 signaling is activated downstream from the prolactin receptor. Other names for JAK2 are JTK10 and THCYT3.

JAK2_{xx-yy}: As used herein this nomenclature refers to a polypeptide fragment of JAK2 consisting of amino acids xx-yy of SEQ ID NO: 5.

JAK2V617F_{xx-yy}: As used herein this nomenclature refers to a polypeptide fragment of JAK2 consisting of amino acids xx-yy of SEQ ID NO: 6.

Myeloproliferative neoplasms (MPNs): The MPNs are acquired hematological cancers, arising from the hematopoietic stem cells, and are characterized by an excessive production of blood cells (essential thrombocythaemia, polycythaemia vera, myelofibrosis) with progressive bone marrow fibrosis leading to bone marrow failure (myelofibrosis) and ultimately acute myelogenous leukemia. The term includes Philadelphia-negative myeloproliferative neoplasms. Essential thrombocythaemia, polycythaemia vera and primary myelofibrosis, may evolve into myelodysplastic syndrome or acute myeloid leukemia. This evolution in a biological continuum from the early cancer stage (ET/PV) to the advanced cancer stage (myelofibrosis with huge splenomegaly) is defined by increasing genomic instability, subclone formation with additional mutations and ultimately resistance to conventional therapies. MHC: Major histocompatibility complex, two main subclasses of MHC, Class I and Class II exist.

Nucleic acid: A chain or sequence of nucleotides that convey genetic information. In regards to the present invention the nucleic acid is generally a deoxyribonucleic acid (DNA).

Nucleic acid construct: A genetically engineered nucleic acid. Typically comprising several elements such as genes or fragments of same, cDNAs, promoters, enhancers, terminators, polyA tails, linkers, polylinkers, operative linkers, multiple cloning sites (MCS), markers, STOP codons, other regulatory elements, internal ribosomal entry sites (IRES) or others.

Operative linker: A sequence of nucleotides or amino acid residues that bind together two parts of a nucleic acid construct or (chimeric) polypeptide in a manner securing the biological processing of the nucleic acid or polypeptide.

PBL: Peripheral blood cells are the cellular components of blood, consisting of red blood cells, white blood cells, and platelets, which are found within the circulating pool of blood and not sequestered within the lymphatic system, spleen, liver, or bone marrow.

PBMC: A Peripheral Blood Mononuclear Cell (PBMC) is a blood cell having a round nucleus, such as a lymphocyte or a monocyte. These blood cells are a critical component in the immune system to fight infection and adapt to intruders. The lymphocyte population consists of T cells (CD4 and CD8 positive ˜75%), B cells and NK cells (˜25% combined).

Polypeptide: Plurality of covalently linked amino acid residues defining a sequence and linked by amide bonds. The term is used analogously with oligopeptide and peptide. The term polypeptide also embraces post-translational modifications introduced by chemical or enzyme-catalyzed reactions, as are known in the art. The term can refer to a variant or fragment of a polypeptide.

Pharmaceutical carriers: also termed excipients, or stabilizers are non-toxic to the cell or individual being exposed thereto at the dosages and concentrations employed. Often the pharmaceutical carrier is an aqueous pH buffered solution. Examples of pharmaceutical carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

Plurality: At least two.

Proliferative disorder: Herein any preneoplastic or neoplastic disease, benign or malignant, where “neoplastic” refers to an abnormal proliferation of cells. A non-limiting example of a proliferative disorder is cancer.

Promoter: A binding site in a DNA chain at which RNA polymerase binds to initiate transcription of messenger RNA by one or more nearby structural genes.

Surfactant: A surface active agent capable of reducing the surface tension of a liquid in which it is dissolved. A surfactant is a compound containing a polar group which is hydrophilic and a non-polar group which is hydrophobic and often composed of a fatty chain.

Treg: Regulatory T cells/T lymphocytes

Treatment: The term “treatment” as used herein may refer to curative treatment and/or to ameliorating treatment and/or to treatment reducing symptoms of disease and/or treatment delaying disease progression.

Vaccine: A substance or composition capable of inducing an immune response in an individual, and particularly in a mammal, preferably in a human being. Also referred to as an immunogenic composition in the present text. A vaccine according to the present invention may frequently be a composition comprising at least an adjuvant and an immunogenically active peptide. An immune response against an agent is a humoral, antibody and/or cellular response inducing memory in an organism, resulting in that said agent is being met by a secondary rather than a primary response, thus reducing its impact on the host organism. Said agent may be pathogen. In the context of the present invention the agent is preferably a cell associated with a proliferative disorder, e.g. a cancer cell. A vaccine of the present invention may be given as a prophylaxis, in order to reduce the risk of encountering a clinical condition and/or as a therapeutic medicament for treatment of a clinical condition. The composition may comprise one or more of the following: antigen(s), nucleic acid constructs encoding one or more antigens, carriers, adjuvants and pharmaceutical carriers.

Variant: a ‘variant’ of a given reference nucleic acid or polypeptide refers to a nucleic acid or polypeptide that displays a certain degree of sequence homology/identity to said reference nucleic acid or polypeptide but is not identical to said reference nucleic acid or polypeptide.

### DETAILED DESCRIPTION OF THE INVENTION

The present disclosure relates to a vaccine composition for use in a method of treatment or prophylaxis of a myeloproliferative disorder.

In another aspect, the disclosure relates to a vaccine composition comprising:
a) one or more of the following:
   (i) an exon 9 mutant of CALR comprising SEQ ID NO: 1 or SEQ ID NO: 16, for example the exon 9 mutant of CALR set forth in SEQ ID NO: 10;
   (ii) an immunogenically active peptide fragment of the exon 9 mutant CALR as set forth in SEQ ID NO: 10, said fragment comprising at least some of amino acids 361 to 411 of SEQ ID NO: 10;
   (iii) an immunogenically active peptide consisting of SEQ ID NO: 16 or SEQ ID NO: 1 or a fragment thereof;
   (iv) a functional homologue of the polypeptides under (i), (ii) or (iii), wherein said functional homologue shares at least 70% sequence identity with SEQ ID NO: 10, and/or said functional homologue is an immunogenically active polypeptide consisting of a sequence identical to a consecutive sequence of amino acids of SEQ ID NO: 16, SEQ ID NO: 1 or SEQ ID NO:10, except that at the most three amino acids have been substituted, such as at the most two amino acids, such as at the most one amino acid;
   (v) a polypeptide comprising any of the polypeptides under (i), (ii), (iii) or (iv);
   (vi) a nucleic acid encoding any of the polypeptides under (i), (ii), (iii), (iv) or (v);

or
a) one or more of the following:
   (vii) the JAK2V617F mutant as set forth in SEQ ID NO: 6;
   (viii) an immunogenically active peptide fragment of the JAK2V617F mutant as set forth in SEQ ID NO: 6, said fragment comprising at least amino acid 617;
   (ix) a functional homologue of the polypeptides under (vii) and (viii), wherein said functional homologue shares at least 70% sequence identity with SEQ ID NO: 6, and/or said functional homologue is an immunogenically active polypeptide consisting of a sequence identical to a consecutive sequence of amino acids of SEQ ID NO: 6, except that at the most three amino acids have been substituted, such as at the most two amino acids, such as at the most one amino acid, wherein the functional homologue comprises at least amino acid 617 of SEQ ID NO:6;
   (x) a polypeptide comprising any of the polypeptides under (vii), (viii) or (ix);
   (xi) a nucleic acid encoding any of the polypeptides under (vii), (viii), (ix) or (x),

said vaccine composition optionally further comprising an adjuvant.

The disclosure also relates to vaccine compositions as described herein for use as a medicament.

The disclosure also relates to a kit-of-parts comprising the vaccine compositions described herein, and a second active ingredient.

In yet another aspect, the disclosure relates to a complex of a peptide fragment as defined herein and a Class I HLA or a Class II HLA molecule or a fragment of such molecule.

In yet another aspect, the disclosure relates to a method of detecting in an individual suffering from a clinical condition the presence of CALR reactive T-cells or JAK2 reactive T-cells, the method comprising contacting a tumor tissue or a blood sample with a complex of the disclosure and detecting binding of the complex to the tissue or the blood cells.

In yet another aspect, a molecule that is capable of binding specifically to a peptide fragment as defined herein is disclosed.

In yet another aspect, a molecule that is capable of blocking the binding of the molecule capable of binding specifically to a peptide fragment as defined herein is disclosed.

In yet another aspect, a method of treating or preventing a clinical condition characterized by the expression of exon 9 mutant CALR and/or the expression of JAK2V617F, the method comprising administering to an individual suffering from said clinical condition an effective amount of the composition, the molecule or peptides described herein is disclosed.

In yet another aspect, the disclosure relates to the use of the vaccine composition, the kit-of-parts, the molecule or the peptide described herein in the manufacture of a medicament for the treatment or prevention of a clinical condition.

In yet another aspect, the disclosure relates to a method of monitoring immunization, said method comprising the steps of
a) providing a blood sample from an individual;
b) providing:
   (i) an exon 9 mutant of CALR comprising SEQ ID NO:16 or SEQ ID NO: 1, for example the exon 9 mutant of CALR set forth in SEQ ID NO:10;
   (ii) an immunogenically active peptide fragment of the exon 9 mutant CALR as set forth in SEQ ID NO: 10, said fragment comprising at least some of amino acids 361 to 411 of SEQ ID NO:10;
   (iii) an immunogenically active peptide consisting of SEQ ID NO:1 or a fragment thereof;
   (iv) the JAK2V617F mutant as set forth in SEQ ID NO: 6;
   (v) an immunogenically active peptide fragment of the JAK2V617F mutant as set forth in SEQ ID NO: 6, said fragment comprising at least amino acid 617 of SEQ ID NO: 6; or
   (vi) a functional homologue of any of the aforementioned; and
c) determining whether said blood sample comprises antibodies or T-cells comprising T-cell receptors specifically binding the protein or peptide thereby determining whether an immune response to said protein or peptide has been raised in said individual.

In yet another aspect, the disclosure relates to an immunogenically active exon 9 mutant CALR peptide fragment comprising at least part of a consecutive sequence of SEQ ID NO: 16 or SEQ ID NO: 1 or a functional homologue thereof to an immunogenically active mutant JAK2 peptide fragment comprising at least part of a consecutive sequence of SEQ ID NO: 7 or a functional homologue thereof, or a nucleic acid encoding said exon 9 mutant CALR peptide fragment or mutant JAK2 peptide fragment for use in the treatment or prevention of clinical conditions associated with expression of mutant CALR or mutant JAK2, such as a myeloproliferative disorder. In such embodiments, said functional homologue is a polypeptide of identical sequence except that at the most three amino acids have been substituted.

Also disclosed is a composition comprising cells which specifically recognize exon 9 mutant CALR or JAK2V617F, wherein preferably the exon 9 mutant CALR comprises a consecutive sequence of SEQ ID NO: 16 or wherein JAK2V617F comprises a consecutive sequence of SEQ ID NO: 6, wherein at the most 3 amino acids are substituted.

In yet another aspect peptides are provided comprising or consisting of up to 50 consecutive amino acids of the sequence of CALR (SEQ ID NO: 9) or of the sequence of an exon 9 mutant of CALR comprising the sequence of SEQ ID NO: 1, wherein said mutant optionally has the sequence of SEQ ID NO: 10, wherein said consecutive amino acids comprise the sequence of any one of SEQ ID NOs: 1, 2, 3, 15, 16, or 17, more preferably wherein said consecutive amino acids comprise SEQ ID NO: 1, SEQ ID NO: 2, SEQ ID NO: 3 or SEQ ID NO: 17, optionally wherein at the most between 1 and 10 consecutive amino acids are replaced by a substitution such as a conservative substitution.

In yet another aspect a composition comprising cells which specifically recognize exon 9 mutant CALR, wherein preferably the exon 9 mutant CALR comprises a consecutive sequence of SEQ ID NO: 16, wherein at the most 3 amino acids are substituted is provided.

In yet another aspect a composition is provided, said composition comprising cells which specifically recognize JAK2V617F, wherein preferably JAK2V617F comprises a consecutive sequence of SEQ ID NO: 6, wherein at the most 3 amino acids are substituted.

In yet another aspect a peptide is provided, said peptide comprising or consisting of up to 50 consecutive amino acids of the sequence of CALR (SEQ ID NO: 9) or of the sequence of an exon 9 mutant of CALR comprising the sequence of SEQ ID NO: 1, wherein said mutant optionally has the sequence of SEQ ID NO: 10, wherein said consecutive amino acids comprise the sequence of any one of SEQ ID NOs: 1, 2, 3, 15, 16, or 17, more preferably wherein said consecutive amino acids comprise SEQ ID NO: 1, SEQ ID NO: 2, SEQ ID NO: 3 or SEQ ID NO: 17, optionally wherein at the most between 1 and 10 consecutive amino acids are replaced by a substitution such as a conservative substitution.

In yet another aspect a peptide is provided, said peptide comprising or consisting of up to 50 consecutive amino acids of the sequence of JAK2 (SEQ ID NO: 5) or of the sequence of JAK2V617F comprising the sequence of SEQ ID NO: 6, wherein said consecutive amino acids comprise the sequence of SEQ ID NO: 7, optionally wherein at the most between 1 and 10 consecutive amino acids are replaced by a substitution such as a conservative substitution.

In yet another aspect pharmaceutical compositions comprising the peptides disclosed herein and optionally a preservative and/or an adjuvant are provided.

In yet another aspect a method of treatment of an individual in need thereof, comprising administering the peptides disclosed herein, is provided.

Vaccine Composition

The present disclosure relates to vaccine compositions for use in methods of treatment or prophylaxis of a proliferative disorder and in particular a myeloproliferative disorder.

It is one aspect of the present invention to provide a vaccine composition comprising:
a) one or more of the following:
   (i) an exon 9 mutant of CALR comprising SEQ ID NO:1, for example the exon 9 mutant of CALR set forth in SEQ ID NO:10;
   (ii) an immunogenically active peptide fragment of the exon 9 mutant CALR as set forth in SEQ ID NO: 10, said fragment comprising at least part of a consecutive sequence of amino acids of SEQ ID NO: 16 or SEQ ID NO: 1;
   (iii) an immunogenically active peptide consisting of SEQ ID NO:1 or a fragment thereof;
   (iv) a functional homologue of the polypeptides under (i), (ii) or (iii), wherein said functional homologue shares at least 70% sequence identity with SEQ ID NO: 10, and/or said functional homologue is an immunogenically active polypeptide consisting of a sequence identical to a consecutive sequence of amino acids of SEQ ID NO: 16, SEQ ID NO: 1 or SEQ ID NO:10, except that at the most three amino acids have been substituted, such as at the most two amino acids, such as at the most one amino acid;
   (v) a polypeptide comprising any of the polypeptides under (i), (ii), (iii) or (iv);
   (vi) a nucleic acid encoding any of the polypeptides under (i), (ii), (iii), (iv) or (v);

or
b) one or more of the following:
   (vii) the JAK2V617F mutant as set forth in SEQ ID NO: 6;
   (viii) an immunogenically active peptide fragment of the JAK2V617F mutant as set forth in SEQ ID NO: 6, said fragment comprising at least amino acid 617;
   (ix) a functional homologue of the polypeptides under (i) and (ii), wherein said functional homologue shares at least 70% sequence identity with SEQ ID NO: 6, and/or said functional homologue is an immunogenically active polypeptide consisting of a sequence identical to a consecutive sequence of amino acids of SEQ ID NO: 6, except that at the most three amino acids have been substituted, such as at the most two amino acids, such as at the most one amino acid;
   (x) a polypeptide comprising any of the polypeptides under (vii), (viii) or (ix);
   (xi) a nucleic acid encoding any of the polypeptides under (vii), (viii), (ix) or (x).

In addition to the above-mentioned said vaccine composition preferably also comprises an adjuvant, which for example may be any of the adjuvants described herein below in the section “Adjuvant”.

Functional homologues, which may be used in the vaccine compositions of the invention, are described herein below in the sections “Calreticulin (CALR)”; “Janus kinase 2 (JAK2)”; “Immunogenically active peptide fragment of exon 9 mutant CALR and JAK2V671F”; “Functional homologues” and “Polypeptides comprising exon 9 mutant CALR and JAK2V671 F or a fragment thereof”.

Calreticulin (CALR)

Calreticulin is a protein (SEQ ID NO: 9) that in humans is encoded by the CALR gene. CALR according to the present disclosure may be any useful CALR. In general it is preferred that CALR is of the same species which it is intended to treat with the vaccine compositions of the disclosure. In preferred embodiments of the disclosure, the vaccine composition is intended for administration to a human being, and hence CALR may be human CALR. The amino acid sequence of wild type human CALR is presented as SEQ ID NO: 9 herein. The full amino acid sequence of the exon 9 mutant of CALR L367fs*46 is presented as SEQ ID NO: 10 herein.

Thus, CALR may be CALR of SEQ ID NO: 9 or a functional homologue thereof sharing at least 70% sequence identity to CALR of SEQ ID NO: 9, said functional homologue preferably having at least 75% sequence identity, for example at least 80% sequence identity, such as at least 85% sequence identity, for example at least 89% sequence identity, such as at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human CALR of SEQ ID NO: 9.

In some embodiments the invention relates to vaccine compositions, methods, cells, peptides or peptide fragments comprising or recognizing an exon 9 mutant CALR of SEQ ID NO: 10 or a functional homologue thereof sharing at least 70% sequence identity to exon 9 mutant CALR of SEQ ID NO: 10, such as a functional homologue preferably having at least 75% sequence identity, for example at least 80% sequence identity, such as at least 85% sequence identity, for example at least 89% sequence identity, such as at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human exon 9 mutant CALR of SEQ ID NO: 10.

Functional homologues of CALR, exon 9 mutant CALR and methods for determining sequence identity are described in more detail in the section “Functional homologues” herein below.

Preferably, the peptide fragment of the exon 9 mutant CALR comprises at least part of a consecutive amino acid sequence of the mutant exon 9 sequence as set forth in SEQ ID NO: 16. In some embodiments, the peptide fragment of the exon 9 mutant CALR comprises or consists of SEQ ID NO: 16, SEQ ID NO: 2, SEQ ID NO: 3, SEQ ID NO: 15, SEQ ID NO: 1 or SEQ ID NO: 17.

In some embodiments the functional homologue of an exon 9 mutant CALR of SEQ ID NO: 10 is a mutant wherein one or more of the amino acids have been mutated to another amino acid or have been deleted. The functional homologue of a peptide fragment of the exon 9 mutant CALR may also be a fragment of SEQ ID NO: 16, SEQ ID NO: 2, SEQ ID NO: 3, SEQ ID NO: 15, SEQ ID NO: 1 or SEQ ID NO: 17, wherein one or more of the amino acids have been mutated to another amino acid or are deleted. In the context of the present invention a “functional homologue” of an exon 9 mutant CALR may lack the catalytic activity of wild type CALR, while retaining the capability to induce an immune response to cells expressing exon 9 mutant CALR.

Janus Kinase 2 (JAK2)

Janus kinase is a protein (SEQ ID NO: 5) that in humans is encoded by the JAK2 gene. JAK2 according to the present disclosure may be any useful JAK2. In general it is preferred that JAK2 is of the same species which it is intended to treat with the vaccine compositions of the disclosure. In preferred embodiments of the disclosure, the vaccine composition is intended for administration to a human being, and hence JAK2 may be human JAK2. The amino acid sequence of wild type human JAK2 is presented as SEQ ID NO: 5 herein. The full amino acid sequence of the JAK2V617F mutant is presented as SEQ ID NO: 6 herein.

Thus, JAK2 may be JAK2 of SEQ ID NO: 5 or a functional homologue thereof sharing at least 70% sequence identity to JAK2 of SEQ ID NO: 5, and accordingly, a functional homologue preferably having at least 75% sequence identity, for example at least 80% sequence identity, such as at least 85% sequence identity, for example at least 89% sequence identity, such as at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human JAK2 of SEQ ID NO: 5.

In some embodiments the invention relates to vaccine compositions, methods, cells, peptides or peptide fragments comprising or recognising the JAK2V617F mutant of SEQ ID NO: 6 or a functional homologue thereof sharing at least 70% sequence identity to JAK2V617F of SEQ ID NO: 6, said functional homologue preferably having at least 75% sequence identity, for example at least 80% sequence identity, such as at least 85% sequence identity, for example at least 89% sequence identity, such as at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human exon 9 mutant JAK2 of SEQ ID NO: 6.

Functional homologues of JAK2, JAK2V617F and methods for determining sequence identity are described in more detail in the section “Functional homologues” herein below.

Preferably, the JAK2V617F fragment comprises at least amino acid 617 of SEQ ID NO: 6. It may also be preferred that the JAK2V617F fragment comprises part of a consecutive amino acid sequence of the JAK2V617F sequence as set forth in SEQ ID NO: 7. In some embodiments, the JAK2V617F fragment is SEQ ID NO: 7.

In some embodiments the JAK2V617F mutant of SEQ ID NO: 6 is a mutant wherein one or more of the amino acids have been mutated to another amino acid or have been deleted. The mutant JAK2V617F may also be a mutant JAK2V617F fragment, for example a fragment of SEQ ID NO: 6, wherein one or more of the amino acids have been mutated to another amino acid or are deleted. In the context of the present invention a “functional homologue” of JAK2V617F may lack the catalytic activity of wild type JAK2, while retaining the capability to induce an immune response to cells expressing JAK2V617F.

Immunogenically Active Peptide Fragments of Exon 9 Mutant CALR and JAK2V617F

The wild-type human CALR, i.e. the naturally occurring, full-length, non-mutated version of the protein, is identified in SEQ ID NO: 9; the wild-type human JAK2, i.e. the naturally occurring, full-length, non-mutated version of the protein, is identified in SEQ ID NO: 5. The present invention covers vaccine compositions comprising CALR; immunologically active peptide fragments of CALR; peptide fragments of CALR, wherein at the most three, such as at the most two, such as at the most one, amino acids have been substituted; and/or functional homologues of CALR comprising a sequence identity of at least 70% to SEQ ID NO: 9.

Preferably, the present invention covers vaccine compositions comprising exon 9 mutant CALR; immunogenically active peptide fragments of exon 9 mutant CALR; peptide fragments of exon 9 mutant CALR, as well as functional homologues thereof, wherein at the most three, such as at the most two, such as at the most one, amino acids have been substituted; and/or functional homologues of exon 9 mutant CALR comprising a sequence identity of at least 70% to SEQ ID NO: 10. Preferably, the exon 9 mutant CALR peptide fragment or functional homologue thereof comprises a consecutive amino acid sequence of SEQ ID NO: 16, SEQ ID NO: 2, SEQ ID NO: 3, SEQ ID NO: 15, SEQ ID NO: 1 or SEQ ID NO: 17. SEQ ID NO: 16 shows the consensus amino acid sequence found in the two most prominent exon 9 mutant CALR, type 1 and type 2. In other words, SEQ ID NO: 16 corresponds to CALR₃₇₆₋₄₁₁ of SEQ ID NO: 10.

The present invention also covers vaccine compositions comprising JAK2; immunologically active peptide fragments of JAK2; peptide fragments of JAK2, as well as functional homologues thereof, wherein at the most three, such as at the most two, such as at the most one, amino acids have been substituted; and/or functional homologues of JAK2 comprising a sequence identity of at least 70% to SEQ ID NO: 5. The present invention also covers vaccine compositions comprising JAK2V617F; immunologically active peptide fragments of JAK2V617F; peptide fragments of JAK2V617F, as well as functional homologues thereof, wherein at the most three, such as at the most two, such as at the most one, amino acids have been substituted; and/or functional homologues of JAK2V617F comprising a sequence identity of at least 70% to SEQ ID NO: 6. Preferably, the JAK2 peptide fragment or functional homologue thereof comprises a consecutive amino acid sequence of SEQ ID NO: 7. SEQ ID NO: 7 shows the amino acid sequence corresponding to JAK2₆₁₀₋₆₁₈ of SEQ ID NO: 6. Preferably, the JAK2 peptide fragment or functional homologue thereof comprises at least amino acid 617 of JAK2V617F of SEQ ID NO: 6.

The term polypeptide fragment is used herein to define any non-full length (as compared to SEQ ID NO: 9, SEQ ID NO: 10, SEQ ID NO: 5 or SEQ ID NO: 6) consecutive sequence of amino acid residues that is directly derived from or synthesized to be identical with at least part of SEQ ID NO: 16, SEQ ID NO: 1, SEQ ID NO: 10 or SEQ ID NO: 6. The peptide fragment may for example be a consecutive sequence of in the range of from 8 to 50, such as in the range of 8 to 40, for example in the range of 8 to 29 amino acids of SEQ ID: 10, such as from 8 to 27 amino acids of SEQ ID NO: 10, for example from 8 to 25 amino acids of SEQ ID NO: 10, for example the peptide fragment may comprise or consists of SEQ ID NO: 16, SEQ ID NO: 1, SEQ ID NO: 2 or SEQ ID NO: 3, or from 20 to 44 amino acids of SEQ ID NO: 10, for example the peptide fragment may comprise or consist of SEQ ID NO: 15, SEQ ID NO: 1 or SEQ ID NO: 17. The peptide fragment may for example be a consecutive sequence of 8 to 50, such as in the range of 8 to 34, for example in the range of 8 to 33, such as 8 to 29 amino acids of SEQ ID: 1. The peptide fragment may for example be a consecutive sequence of in the range of from 8 to 50, for example in the range of 8 to 44, such as in the range of 8 to 40, for example in the range of 8 to 29 amino acids of SEQ ID: 10, such as from 8 to 27 amino acids of SEQ ID NO: 10, for example from 8 to 25 amino acids of SEQ ID NO: 10, for example the peptide fragment may comprise or consists of SEQ ID NO: 16, SEQ ID NO: 2, SEQ ID NO: 3, SEQ ID NO: 15, SEQ ID NO: 1 or SEQ ID NO: 17. The peptide fragment may for example be a consecutive sequence of 8 to 50, such as in the range of 8 to 44, such as in the range of 8 to 34, for example in the range of 8 to 33, such as 8 to 29 amino acids of any of the exon 9 CALR mutants L367fs*46 (full length set forth in SEQ ID NO: 10), E370fs*43, E370fs*48, L367fs*48, L367fs*44, K368fs*51, L367fs*52, R366fs*53, E371fs*49, K368fs*43, E370fs*37, D373fs*47, K374fs*53, E371fs*49, K385fs*47, K385fs*47, R376fs*55, K385fs*47, E381fs*48 (Nangalia et al., 2013; the sequences of the above mutations are listed in FIG. 3, panel A, p. 2400 of Nangalia et al.).

The peptide fragment may for example be a consecutive sequence of in the range of from 8 to 50, such as in the range of 8 to 40, for example in the range of 8 to 29 amino acids of SEQ ID: 6, such as from 8 to 27 amino acids of SEQ ID NO: 6, for example from 8 to 25 amino acids of SEQ ID NO: 6, for example the peptide fragment may comprise or consist of SEQ ID NO: 7.

A functional homologue of wild-type CALR can be defined as a full length or fragment of CALR that differs in sequence from the wild-type CALR, such as wild-type human CALR of SEQ ID NO: 9, but is still capable of inducing an immune response against cells expressing mutant CALR such as proliferative neoplasm cells and DCs. In some embodiments, the CALR expressed in these cells is an exon 9 mutant of CALR, for example as set forth in SEQ ID NO: 10.

A functional homologue of an exon 9 CALR mutant can be defined as a full length or fragment of an exon 9 CALR mutant that differs in sequence from the exon 9 CALR mutant of SEQ ID NO: 10, but is still capable of inducing an immune response against cells expressing mutant CALR such as proliferative neoplasm cells and DCs. In some embodiments, the exon 9 mutant CALR expressed in these cells is the exon 9 mutant of CALR as set forth in SEQ ID NO: 10. In one embodiment, the fragment of an exon 9 CALR mutant that is capable of inducing an immune response against cells expressing mutant CALR such as proliferative neoplasm cells and DCs comprises or consists of the fragment set forth in SEQ ID NO: 16. In another embodiment, the fragment comprises or consists of the fragment set forth in SEQ ID NO: 2. In another embodiment, the fragment comprises or consists of the fragment set forth in SEQ ID NO: 3. In another embodiment, the fragment comprises or consists of the fragment set forth in SEQ ID NO: 15. In another embodiment, the fragment comprises or consists of the fragment set forth in SEQ ID NO: 1. In another embodiment, the fragment comprises or consists of the fragment set forth in SEQ ID NO: 17.

A functional homologue of wild-type JAK2 can be defined as a full length or fragment of wild-type JAK2 that differs in sequence from the wild-type JAK2, such as wild-type human JAK2 of SEQ ID NO: 5, but is still capable of inducing an immune response against cells expressing JAK2V617F of SEQ ID NO: 6 such as proliferative neoplasm cells and DCs.

A functional homologue of a JAK2V617F mutant can be defined as a full length or fragment of JAK2V617F that differs in sequence from JAK2V617F as set forth in SEQ ID NO: 6, but is still capable of inducing an immune response against cells expressing JAK2V617F of SEQ ID NO: 6 such as proliferative neoplasm cells and DCs. Preferably, the functional homologue comprises at least amino acid 617 of SEQ ID NO: 6.

A functional homologue may be a mutated version or an alternative splice variant of the wild-type CALR or JAK2. In another aspect, functional homologues of CALR or JAK2 are defined as described herein below. A functional homologue may be, but is not limited to, a recombinant version of full length or fragmented CALR, exon 9 CALR mutant, JAK2 or JAK2V617F mutant with one or more mutations and/or one or more sequence deletions and/or additions introduced ex vivo.

Accordingly, in one embodiment the immunogenically active exon 9 mutant CALR peptide fragment of the invention consists of at the most 90 consecutive amino acid residues, such as at the most 80 consecutive amino acids residues, for example at the most 70 consecutive amino acid residues, such as at the most 60 consecutive amino acid residues, for example at the most 50 consecutive amino acid residues, for example at the most 45 consecutive amino acid residues, such as at the most 40 consecutive amino acid residues, for example at the most 35 consecutive amino acid residues, such as at the most 30 consecutive amino acid residues, for example at the most 29 consecutive amino acid residues, such as at the most 24 consecutive amino acid residues, such as at the most 22 consecutive amino acid residues, such as at the most 20 consecutive amino acid residues, such as at the most 15 consecutive amino acid residues, such as at the most 10 consecutive amino acid residues, such as at the most 9 consecutive amino acid residues, such as at the most 8 consecutive amino acid residues of exon 9 mutant CALR as identified in SEQ ID NO: 10 or a functional homologue thereof; the functional homologue being a polypeptide of identical sequence except that at the most three amino acids have been substituted, such as at the most two amino acids have been substituted, such as at the most one amino acid has been substituted. The substitution may be a conservative substitution.

Said immunogenically active peptide exon 9 mutant CALR fragment may also consist of at the most 80 consecutive amino acids residues, for example at the most 70 consecutive amino acid residues, such as at the most 60 consecutive amino acid residues, for example at the most 50 consecutive amino acid residues, for example at the most 45 consecutive amino acid residues, such as at the most 40 consecutive amino acid residues, for example at the most 35 consecutive amino acid residues, such as at the most 30 consecutive amino acid residues, for example at the most 29 consecutive amino acid residues, such as at the most 24 consecutive amino acid residues, such as at the most 22 consecutive amino acid residues, such as at the most 20 consecutive amino acid residues of exon 9 mutant CALR as identified in SEQ ID NO: 10, such as 24 to 32 consecutive amino acid residues, such as 26 to 29 consecutive amino acid residues of exon 9 mutant CALR as identified in SEQ ID NO: 10, wherein one or more amino acids have been mutated to another amino acid or deleted.

In one preferred embodiment of the invention, the immunogenically active peptide fragment consists of in the range of 20 to 29 amino acids, preferably of 29 consecutive amino acids of SEQ ID NO: 16, SEQ ID NO: 2, SEQ ID NO: 3, SEQ ID NO: 15, SEQ ID NO: 1 or SEQ ID NO: 17 or a functional homologue thereof; the functional homologue being a polypeptide of identical sequence except that at the most three amino acids have been substituted, such as at the most two amino acids have been substituted, such as at the most one amino acid has been substituted.

Accordingly in another specific embodiment the immunogenically active exon 9 mutant CALR peptide fragment of the invention consists of at the most 34 amino acid residues, for example at the most 33 amino acid residues, such as at the most 32 amino acid residues, for example at the most 31 amino acid residues, such as at the most 30 amino acid residues, for example at the most 29 amino acid residues, such as at the most 28 amino acid residues, for example at the most 27 amino acid residues, such as at the most 26 amino acid residues, for example at the most 25 amino acid residues, such as at the most 24 amino acid residues, for example at the most 23 amino acid residues, such as at the most 22 amino acid residues, for example at the most 21 amino acid residues, such as at the most 20 amino acid residues, for example at the most 19 amino acid residues, such as at the most 18 amino acid residues, such as at the most 17 amino acid residues, for example at the most 16 amino acid residues, such as at the most 15 amino acid residues, for example at the most 14 amino acid residues, such as at the most 13 amino acid residues, for example at the most 12 amino acid residues, such as at the most 11 amino acid residues, such as 8 to 10 amino acid residues, such as 9 to 10 consecutive amino acids residues from SEQ ID NO: 16 or a functional homologue thereof; the functional homologue being a polypeptide of identical sequence except that at the most three amino acids have been substituted, such as at the most two amino acids have been substituted, such as at the most one amino acid has been substituted.

In another specific embodiment the immunogenically active exon 9 mutant CALR peptide fragment of the invention consists of at the most 29 amino acid residues, such as at the most 28 amino acid residues, for example at the most 27 amino acid residues, such as at the most 26 amino acid residues, for example at the most 25 amino acid residues, such as at the most 24 amino acid residues, for example at the most 23 amino acid residues, such as at the most 22 amino acid residues, for example at the most 21 amino acid residues, such as at the most 20 amino acid residues, for example at the most 19 amino acid residues, such as at the most 18 amino acid residues, such as at the most 17 amino acid residues, for example at the most 16 amino acid residues, such as at the most 15 amino acid residues, for example at the most 14 amino acid residues, such as at the most 13 amino acid residues, for example at the most 12 amino acid residues, such as at the most 11 amino acid residues, such as 8 to 10 amino acid residues, such as 9 to 10 consecutive amino acids residues from SEQ ID NO: 2 or a functional homologue thereof; the functional homologue being a polypeptide of identical sequence except that at the most three amino acids have been substituted, such as at the most two amino acids have been substituted, such as at the most one amino acid has been substituted.

In another specific embodiment the immunogenically active exon 9 mutant CALR peptide fragment of the invention consists of at the most 29 amino acid residues, such as at the most 28 amino acid residues, for example at the most 27 amino acid residues, such as at the most 26 amino acid residues, for example at the most 25 amino acid residues, such as at the most 24 amino acid residues, for example at the most 23 amino acid residues, such as at the most 22 amino acid residues, for example at the most 21 amino acid residues, such as at the most 20 amino acid residues, for example at the most 19 amino acid residues, such as at the most 18 amino acid residues, such as at the most 17 amino acid residues, for example at the most 16 amino acid residues, such as at the most 15 amino acid residues, for example at the most 14 amino acid residues, such as at the most 13 amino acid residues, for example at the most 12 amino acid residues, such as at the most 11 amino acid residues, such as 8 to 10 amino acid residues, such as 9 to 10 consecutive amino acids residues from SEQ ID NO: 3 or a functional homologue thereof; the functional homologue being a polypeptide of identical sequence except that at the most three amino acids have been substituted, such as at the most two amino acids have been substituted, such as at the most one amino acid has been substituted.

In another specific embodiment the immunogenically active exon 9 mutant CALR peptide fragment of the invention consists of at the most 20 amino acid residues, for example at the most 19 amino acid residues, such as at the most 18 amino acid residues, such as at the most 17 amino acid residues, for example at the most 16 amino acid residues, such as at the most 15 amino acid residues, for example at the most 14 amino acid residues, such as at the most 13 amino acid residues, for example at the most 12 amino acid residues, such as at the most 11 amino acid residues, such as 8 to 10 amino acid residues, such as 9 to 10 consecutive amino acids residues from SEQ ID NO: 15 or a functional homologue thereof; the functional homologue being a polypeptide of identical sequence except that at the most three amino acids have been substituted, such as at the most two amino acids have been substituted, such as at the most one amino acid has been substituted.

In another specific embodiment the immunogenically active exon 9 mutant CALR peptide fragment of the invention consists of at the most 36 amino acid residues, for example at the most 35 amino acid residues, such as at the most 34 amino acid residues, for example at the most 33 amino acid residues, such as at the most 32 amino acid residues, for example at the most 31 amino acid residues, such as at the most 30 amino acid residues, for example at the most 29 amino acid residues, such as at the most 28 amino acid residues, for example at the most 27 amino acid residues, such as at the most 26 amino acid residues, for example at the most 25 amino acid residues, such as at the most 24 amino acid residues, for example at the most 23 amino acid residues, such as at the most 22 amino acid residues, for example at the most 21 amino acid residues, such as at the most 20 amino acid residues, for example at the most 19 amino acid residues, such as at the most 18 amino acid residues, such as at the most 17 amino acid residues, for example at the most 16 amino acid residues, such as at the most 15 amino acid residues, for example at the most 14 amino acid residues, such as at the most 13 amino acid residues, for example at the most 12 amino acid residues, such as at the most 11 amino acid residues, such as 8 to 10 amino acid residues, such as 9 to 10 consecutive amino acids residues from SEQ ID NO: 1 or a functional homologue thereof; the functional homologue being a polypeptide of identical sequence except that at the most three amino acids have been substituted, such as at the most two amino acids have been substituted, such as at the most one amino acid has been substituted.

In another specific embodiment the immunogenically active exon 9 mutant CALR peptide fragment of the invention consists of at the most 44 amino acid residues, for example at the most 43 amino acid residues, such as at the most 42 amino acid residues, for example at the most 41 amino acid residues, such as 40 amino acid residues, for example at the most 39 amino acid residues, such as at the most 38 amino acid residues, for example at the most 37 amino acid residues, such as at the most 36 amino acid residues, for example at the most 35 amino acid residues, such as at the most 34 amino acid residues, for example at the most 33 amino acid residues, such as at the most 32 amino acid residues, for example at the most 31 amino acid residues, such as at the most 30 amino acid residues, for example at the most 29 amino acid residues, such as at the most 28 amino acid residues, for example at the most 27 amino acid residues, such as at the most 26 amino acid residues, for example at the most 25 amino acid residues, such as at the most 24 amino acid residues, for example at the most 23 amino acid residues, such as at the most 22 amino acid residues, for example at the most 21 amino acid residues, such as at the most 20 amino acid residues, for example at the most 19 amino acid residues, such as at the most 18 amino acid residues, such as at the most 17 amino acid residues, for example at the most 16 amino acid residues, such as at the most 15 amino acid residues, for example at the most 14 amino acid residues, such as at the most 13 amino acid residues, for example at the most 12 amino acid residues, such as at the most 11 amino acid residues, such as 8 to 10 amino acid residues, such as 9 to 10 consecutive amino acids residues from SEQ ID NO: 17 or a functional homologue thereof; the functional homologue being a polypeptide of identical sequence except that at the most three amino acids have been substituted, such as at the most two amino acids have been substituted, such as at the most one amino acid has been substituted.

In one preferred embodiment of the invention, the immunogenically active CALR peptide comprises at the most 29 consecutive amino acid residues from exon 9 mutant CALR, such as at the most 28 consecutive amino acid residues, such as 27 consecutive amino acid residues, such as 26 consecutive amino acid residues from an exon 9 mutant CALR comprising SEQ ID NO:1 or from the exon 9 mutant CALR fragment identified in SEQ ID NO: 16, SEQ ID NO: 2 or SEQ ID NO: 3, or a functional homologue thereof; the functional homologue being a polypeptide of identical sequence except that at the most three amino acids have been substituted, such as at the most two amino acids have been substituted, such as at the most one amino acid has been substituted. In another embodiment, the immunogenically active CALR peptide comprises at the most 44 consecutive amino acid residues from exon 9 mutant CALR, such as at the most 36 consecutive amino acid residues, such as 20 consecutive amino acid residues from an exon 9 mutant CALR comprising SEQ ID NO:1 or from the exon 9 mutant CALR fragment identified in SEQ ID NO: 15, SEQ ID NO: 1 or SEQ ID NO: 17, or a functional homologue thereof; the functional homologue being a polypeptide of identical sequence except that at the most three amino acids have been substituted, such as at the most two amino acids have been substituted, such as at the most one amino acid has been substituted.

In another embodiment the immunogenically active exon 9 mutant CALR peptide fragment of the invention consists of at least 8 amino acid residues, such as at least 9 amino acid residues, for example at least 10 amino acid residues, such as at least 11 amino acid residues, for example at least 12 amino acid residues, such as at least 13 amino acid residues, for example at least 14 amino acid residues, such as at least 15 amino acid residues, for example at least 16 amino acid residues, such as at least 17 amino acid residues, for example at least 18 amino acid residues, such as at least 19 amino acid residues, for example at least 20 amino acid residues, such as at least 21 amino acid residues, for example at least 22 amino acid residues, such as at least 23 amino acid residues, for example at least 24 amino acid residues, such as at least 25 amino acid residues, for example at least 26 amino acid residues, such as at least 27 amino acid residues, for example at least 28 amino acid residues, such as at least 29 amino acid residues, for example at least 30 amino acid residues, such as at least 31 amino acid residues, for example at least 32 amino acid residues, such as at least 33 amino acid residues, for example at least 34 amino acid residues, such as at least 35 amino acid residues, for example at least 36 amino acid residues, such as at least 37 amino acid residues, for example at least 38 amino acid residues, such as at least 39 amino acid residues, for example at least 40 amino acid residues, such as at least 41 amino acid residues, for example at least 42 amino acid residues, such as at least 43 amino acid residues, for example at least 44 amino acid residues, such as 25 to 35 amino acid residues, such as 20 to 36 amino acid residues, such as 29 to 44 amino acid residues, such as 26 to 32 consecutive amino acids residues from SEQ ID NO: 16 or a functional homologue thereof; the functional homologue being a polypeptide of identical sequence except that at the most three amino acids have been substituted, such as at the most two amino acids have been substituted, such as at the most one amino acid has been substituted.

In another embodiment the immunogenically active exon 9 mutant CALR peptide fragment of the invention consists of at least 8 amino acid residues, such as at least 9 amino acid residues, for example at least 10 amino acid residues, such as at least 11 amino acid residues, for example at least 12 amino acid residues, such as at least 13 amino acid residues, for example at least 14 amino acid residues, such as at least 15 amino acid residues, for example at least 16 amino acid residues, such as at least 17 amino acid residues, for example at least 18 amino acid residues, such as at least 19 amino acid residues, for example at least 20 amino acid residues, such as at least 21 amino acid residues, for example at least 22 amino acid residues, such as at least 23 amino acid residues, for example at least 24 amino acid residues, such as at least 25 amino acid residues, for example at least 26 amino acid residues, such as at least 27 amino acid residues, for example at least 28 amino acid residues, such as 29 amino acid residues, such as 25 to 29 amino acid residues, such as 26 to 29 consecutive amino acids residues from SEQ ID NO: 2 or a functional homologue thereof; the functional homologue being a polypeptide of identical sequence except that at the most three amino acids have been substituted, such as at the most two amino acids have been substituted, such as at the most one amino acid has been substituted.

In another specific embodiment the immunogenically active exon 9 mutant CALR peptide fragment of the invention consists of at least 8 amino acid residues, such as at least 9 amino acid residues, for example at least 10 amino acid residues, such as at least 11 amino acid residues, for example at least 12 amino acid residues, such as at least 13 amino acid residues, for example at least 14 amino acid residues, such as at least 15 amino acid residues, for example at least 16 amino acid residues, such as at least 17 amino acid residues, for example at least 18 amino acid residues, such as at least 19 amino acid residues, for example at least 20 amino acid residues, such as at least 21 amino acid residues, for example at least 22 amino acid residues, such as at least 23 amino acid residues, for example at least 24 amino acid residues, such as at least 25 amino acid residues, for example at least 26 amino acid residues, such as at least 27 amino acid residues, for example at least 28 amino acid residues, such as 29 amino acid residues, such as 25 to 29 amino acid residues, such as 26 to 29 consecutive amino acids residues from SEQ ID NO: 3 or a functional homologue thereof; the functional homologue being a polypeptide of identical sequence except that at the most three amino acids have been substituted, such as at the most two amino acids have been substituted, such as at the most one amino acid has been substituted.

In another specific embodiment the immunogenically active exon 9 mutant CALR peptide fragment of the invention consists of at least 8 amino acid residues, such as at least 9 amino acid residues, for example at least 10 amino acid residues, such as at least 11 amino acid residues, for example at least 12 amino acid residues, such as at least 13 amino acid residues, for example at least 14 amino acid residues, such as at least 15 amino acid residues, for example at least 16 amino acid residues, such as at least 17 amino acid residues, for example at least 18 amino acid residues, such as at least 19 amino acid residues, for example 20 consecutive amino acids residues from SEQ ID NO: 15 or a functional homologue thereof; the functional homologue being a polypeptide of identical sequence except that at the most three amino acids have been substituted, such as at the most two amino acids have been substituted, such as at the most one amino acid has been substituted.

In another specific embodiment the immunogenically active exon 9 mutant CALR peptide fragment of the invention consists of at least 8 amino acid residues, such as at least 9 amino acid residues, for example at least 10 amino acid residues, such as at least 11 amino acid residues, for example at least 12 amino acid residues, such as at least 13 amino acid residues, for example at least 14 amino acid residues, such as at least 15 amino acid residues, for example at least 16 amino acid residues, such as at least 17 amino acid residues, for example at least 18 amino acid residues, such as at least 19 amino acid residues, for example at least 20 amino acid residues, such as at least 21 amino acid residues, for example at least 22 amino acid residues, such as at least 23 amino acid residues, for example at least 24 amino acid residues, such as at least 25 amino acid residues, for example at least 26 amino acid residues, such as at least 27 amino acid residues, for example at least 28 amino acid residues, such as at least 29 amino acid residues, for example at least 30 amino acid residues, such as at least 31 amino acid residues, for example at least 32 amino acid residues, such as at least 33 amino acid residues, for example at least 34 amino acid residues, such as at least 35 amino acid residues, such as 36 amino acid residues, such as 20 to 36 amino acid residues, such as 29 to 36 consecutive amino acids residues from SEQ ID NO: 1 or a functional homologue thereof; the functional homologue being a polypeptide of identical sequence except that at the most three amino acids have been substituted, such as at the most two amino acids have been substituted, such as at the most one amino acid has been substituted.

In another specific embodiment the immunogenically active exon 9 mutant CALR peptide fragment of the invention consists of at least 8 amino acid residues, such as at least 9 amino acid residues, for example at least 10 amino acid residues, such as at least 11 amino acid residues, for example at least 12 amino acid residues, such as at least 13 amino acid residues, for example at least 14 amino acid residues, such as at least 15 amino acid residues, for example at least 16 amino acid residues, such as at least 17 amino acid residues, for example at least 18 amino acid residues, such as at least 19 amino acid residues, for example at least 20 amino acid residues, such as at least 21 amino acid residues, for example at least 22 amino acid residues, such as at least 23 amino acid residues, for example at least 24 amino acid residues, such as at least 25 amino acid residues, for example at least 26 amino acid residues, such as at least 27 amino acid residues, for example at least 28 amino acid residues, such as at least 29 amino acid residues, for example at least 30 amino acid residues, such as at least 31 amino acid residues, for example at least 32 amino acid residues, such as at least 33 amino acid residues, for example at least 34 amino acid residues, such as at least 35 amino acid residues, for example at least 36 amino acid residues, such as at least 37 amino acid residues, for example at least 38 amino acid residues, such as at least 39 amino acid residues, for example at least 40 amino acid residues, such as at least 41 amino acid residues, for example at least 42 amino acid residues, such as at least 43 amino acid residues, for example 44 amino acid residues, such as 20 to 44 amino acid residues, such as 29 to 44 amino acid residues, such as 36 to 44 consecutive amino acids residues from SEQ ID NO: 17 or a functional homologue thereof; the functional homologue being a polypeptide of identical sequence except that at the most three amino acids have been substituted, such as at the most two amino acids have been substituted, such as at the most one amino acid has been substituted.

In one preferred embodiment of the invention, the immunogenically active CALR peptide comprises at least 25 consecutive amino acid residues from exon 9 mutant CALR, such as at least 26 consecutive amino acid residues, such as at least 27 consecutive amino acid residues, such as at least 28 consecutive amino acid residues, such as 29 consecutive amino acid residues from an exon 9 mutant CALR comprising SEQ ID NO:1 or from the exon 9 mutant CALR fragment identified in SEQ ID NO: 16, SEQ ID NO: 2, SEQ ID NO: 3, SEQ ID NO: 15, SEQ ID NO: 1 or SEQ ID NO: 17, or a functional homologue thereof; the functional homologue being a polypeptide of identical sequence except that at the most three amino acids have been substituted, such as at the most two amino acids have been substituted, such as at the most one amino acid has been substituted.

In particular, the immunogenically active exon 9 mutant CALR peptide may consist of in the range of 8 to 34, such as in the range of 8 to 30, for example in the range of 8 to 29 consecutive amino acid residues from SEQ ID NO: 16 or the immunogenically active peptide may consist of 28 consecutive amino acid residues from the exon 9 mutant CALR fragment of SEQ ID NO: 16. In some embodiments, the immunogenically peptide comprises at the most 29 consecutive amino acid residues from a fragment of an exon 9 mutant CALR, such as at the most 28 consecutive amino acid residues, such as 27 consecutive amino acid residues, such as 26 consecutive amino acid residues from a fragment of an exon 9 mutant CALR as identified in SEQ ID NO: 16 or a functional homologue thereof; the functional homologue being a polypeptide of identical sequence except that at the most three amino acids have been substituted, such as at the most two amino acids have been substituted, such as at the most one amino acid has been substituted. In other embodiments, the immunogenically peptide comprises at least 28 consecutive amino acid residues from a fragment of an exon 9 mutant CALR, such as at least 29 consecutive amino acid residues, such as at least 30 consecutive amino acid residues, such as 31 consecutive amino acid residues, such as 32 consecutive amino acid residues from a fragment of an exon 9 mutant CALR as identified in SEQ ID NO: 16 or a functional homologue thereof; the functional homologue being a polypeptide of identical sequence except that at the most three amino acids have been substituted, such as at the most two amino acids have been substituted, such as at the most one amino acid has been substituted. In particular, the immunogenically active peptide may consist of 29 consecutive amino acid residues from the exon 9 mutant CALR fragment of SEQ ID NO: 16 or the immunogenically active peptide may consist of 28 consecutive amino acid residues from the exon 9 mutant CALR fragment of SEQ ID NO: 16.

In another embodiment, the immunogenically active CALR peptide may consist of in the range of 8 to 29 consecutive amino acid residues from the exon 9 mutant CALR fragment of SEQ ID NO: 2 or the immunogenically active peptide may consist of in the range of 25 to 29 consecutive amino acid residues from the exon 9 mutant CALR fragment of SEQ ID NO: 2. In some embodiments, the immunogenically peptide comprises at the most 29 consecutive amino acid residues from a fragment of an exon 9 mutant CALR, such as at the most 28 consecutive amino acid residues, such as 27 consecutive amino acid residues, such as 26 consecutive amino acid residues from a fragment of an exon 9 mutant CALR as identified in SEQ ID NO: 2 or a functional homologue thereof; the functional homologue being a polypeptide of identical sequence except that at the most three amino acids have been substituted, such as at the most two amino acids have been substituted, such as at the most one amino acid has been substituted. In other embodiments, the immunogenically peptide comprises at least 25 consecutive amino acid residues from a fragment of an exon 9 mutant CALR, such as at least 26 consecutive amino acid residues, such as 27 consecutive amino acid residues, such as 28 consecutive amino acid residues, such as 29 consecutive amino acid residues from a fragment of an exon 9 mutant CALR as identified in SEQ ID NO: 2 or a functional homologue thereof; the functional homologue being a polypeptide of identical sequence except that at the most three amino acids have been substituted, such as at the most two amino acids have been substituted, such as at the most one amino acid has been substituted. In particular, the immunogenically active peptide may consist of 29 consecutive amino acid residues from the CALR fragment of SEQ ID NO: 2 or the immunogenically active peptide may consist of 28 consecutive amino acid residues from the CALR fragment of SEQ ID NO: 2.

In another embodiment, the immunogenically active CALR peptide may consist of in the range of 8 to 29 consecutive amino acid residues from the exon 9 mutant CALR fragment of SEQ ID NO: 3 or the immunogenically active peptide may consist of in the range of 25 to 29 consecutive amino acid residues from the exon 9 mutant CALR fragment of SEQ ID NO: 3. In some embodiments, the immunogenically peptide comprises at the most 29 consecutive amino acid residues from a fragment of an exon 9 mutant CALR, such as at the most 28 consecutive amino acid residues, such as 27 consecutive amino acid residues, such as 26 consecutive amino acid residues from a fragment of an exon 9 mutant CALR as identified in SEQ ID NO: 3 or a functional homologue thereof; the functional homologue being a polypeptide of identical sequence except that at the most three amino acids have been substituted, such as at the most two amino acids have been substituted, such as at the most one amino acid has been substituted. In other embodiments, the immunogenically peptide comprises at least 25 consecutive amino acid residues from a fragment of an exon 9 mutant CALR, such as at least 26 consecutive amino acid residues, such as 27 consecutive amino acid residues, such as 28 consecutive amino acid residues, such as 29 consecutive amino acid residues from a fragment of an exon 9 mutant CALR as identified in SEQ ID NO: 3 or a functional homologue thereof; the functional homologue being a polypeptide of identical sequence except that at the most three amino acids have been substituted, such as at the most two amino acids have been substituted, such as at the most one amino acid has been substituted. In particular, the immunogenically active peptide may consist of 29 consecutive amino acid residues from the CALR fragment of SEQ ID NO: 3 or the immunogenically active peptide may consist of 28 consecutive amino acid residues from the CALR fragment of SEQ ID NO: 3.

In another embodiment, the immunogenically active CALR peptide may consist of in the range of 8 to 20 consecutive amino acid residues from the exon 9 mutant CALR fragment of SEQ ID NO: 15 or the immunogenically active peptide may consist of in the range of 15 to 20 consecutive amino acid residues from the exon 9 mutant CALR fragment of SEQ ID NO: 15. In some embodiments, the immunogenically peptide comprises at the most 20 consecutive amino acid residues from a fragment of an exon 9 mutant CALR, such as at the most 19 consecutive amino acid residues, such as 18 consecutive amino acid residues, such as 17 consecutive amino acid residues from a fragment of an exon 9 mutant CALR as identified in SEQ ID NO: 15 or a functional homologue thereof; the functional homologue being a polypeptide of identical sequence except that at the most three amino acids have been substituted, such as at the most two amino acids have been substituted, such as at the most one amino acid has been substituted. In other embodiments, the immunogenically peptide comprises at least 16 consecutive amino acid residues from a fragment of an exon 9 mutant CALR, such as at least 17 consecutive amino acid residues, such as 18 consecutive amino acid residues, such as 19 consecutive amino acid residues, such as 20 consecutive amino acid residues from a fragment of an exon 9 mutant CALR as identified in SEQ ID NO: 15 or a functional homologue thereof; the functional homologue being a polypeptide of identical sequence except that at the most three amino acids have been substituted, such as at the most two amino acids have been substituted, such as at the most one amino acid has been substituted. In particular, the immunogenically active peptide may consist of 20 consecutive amino acid residues from the CALR fragment of SEQ ID NO: 15 or the immunogenically active peptide may consist of 15 consecutive amino acid residues from the CALR fragment of SEQ ID NO: 15.

In another embodiment, the immunogenically active CALR peptide may consist of in the range of 30 to 36 consecutive amino acid residues from the exon 9 mutant CALR fragment of SEQ ID NO: 1 or the immunogenically active peptide may consist of in the range of 15 to 20 consecutive amino acid residues from the exon 9 mutant CALR fragment of SEQ ID NO: 1. In some embodiments, the immunogenically peptide comprises at the most 36 consecutive amino acid residues from a fragment of an exon 9 mutant CALR, such as at the most 35 consecutive amino acid residues, such as 34 consecutive amino acid residues, such as 33 consecutive amino acid residues from a fragment of an exon 9 mutant CALR as identified in SEQ ID NO: 1 or a functional homologue thereof; the functional homologue being a polypeptide of identical sequence except that at the most three amino acids have been substituted, such as at the most two amino acids have been substituted, such as at the most one amino acid has been substituted. In other embodiments, the immunogenically peptide comprises at least 30 consecutive amino acid residues from a fragment of an exon 9 mutant CALR, such as at least 33 consecutive amino acid residues, such as 34 consecutive amino acid residues, such as 35 consecutive amino acid residues, such as 36 consecutive amino acid residues from a fragment of an exon 9 mutant CALR as identified in SEQ ID NO: 1 or a functional homologue thereof; the functional homologue being a polypeptide of identical sequence except that at the most three amino acids have been substituted, such as at the most two amino acids have been substituted, such as at the most one amino acid has been substituted. In particular, the immunogenically active peptide may consist of 36 consecutive amino acid residues from the CALR fragment of SEQ ID NO: 1 or the immunogenically active peptide may consist of 30 consecutive amino acid residues from the CALR fragment of SEQ ID NO: 1.

In another embodiment, the immunogenically active CALR peptide may consist of in the range of 37 to 44 consecutive amino acid residues from the exon 9 mutant CALR fragment of SEQ ID NO: 17 or the immunogenically active peptide may consist of in the range of 30 to 35 consecutive amino acid residues from the exon 9 mutant CALR fragment of SEQ ID NO: 17. In some embodiments, the immunogenically peptide comprises at the most 44 consecutive amino acid residues from a fragment of an exon 9 mutant CALR, such as at the most 43 consecutive amino acid residues, such as 42 consecutive amino acid residues, such as 41 consecutive amino acid residues from a fragment of an exon 9 mutant CALR as identified in SEQ ID NO: 17 or a functional homologue thereof; the functional homologue being a polypeptide of identical sequence except that at the most three amino acids have been substituted, such as at the most two amino acids have been substituted, such as at the most one amino acid has been substituted. In other embodiments, the immunogenically peptide comprises at least 37 consecutive amino acid residues from a fragment of an exon 9 mutant CALR, such as at least 38 consecutive amino acid residues, such as 39 consecutive amino acid residues, such as 40 consecutive amino acid residues, such as 41 consecutive amino acid residues from a fragment of an exon 9 mutant CALR as identified in SEQ ID NO: 17 or a functional homologue thereof; the functional homologue being a polypeptide of identical sequence except that at the most three amino acids have been substituted, such as at the most two amino acids have been substituted, such as at the most one amino acid has been substituted. In particular, the immunogenically active peptide may consist of 44 consecutive amino acid residues from the CALR fragment of SEQ ID NO: 17 or the immunogenically active peptide may consist of 36 consecutive amino acid residues from the CALR fragment of SEQ ID NO: 17.

In another embodiment of the invention, the immunogenically active exon 9 mutant CALR peptide comprises at the most 15 consecutive amino acid residues from the CALR peptide fragment of SEQ ID NO: 16, such as at the most 12 consecutive amino acid residues, such as at the most 11 consecutive amino acid residues, such as at the most 10 consecutive amino acid residues, such as at the most 9 consecutive amino acid residues, such as 8 consecutive amino acid residues, from the exon 9 mutant CALR peptide fragment as identified in SEQ ID NO: 16 or a functional homologue thereof; the functional homologue being a polypeptide of identical sequence except that at the most three amino acids have been substituted, such as at the most two amino acids have been substituted, such as at the most one amino acid has been substituted.

In another embodiment of the invention, the immunogenically active exon 9 mutant CALR peptide comprises at least 25 consecutive amino acid residues from the CALR peptide fragment of SEQ ID NO: 16, such as at least 26 consecutive amino acid residues, such as at least 27 consecutive amino acid residues, such as a at least 28 consecutive amino acid residues, such as at least 29 consecutive amino acid residues, such at least 30 consecutive amino acid residues, from the exon 9 mutant CALR peptide fragment as identified in SEQ ID NO: 16 or a functional homologue thereof; the functional homologue being a polypeptide of identical sequence except that at the most three amino acids have been substituted, such as at the most two amino acids have been substituted, such as at the most one amino acid has been substituted.

In another embodiment of the invention, the immunogenically active exon 9 mutant CALR peptide comprises at the most 15 consecutive amino acid residues from SEQ ID NO: 2, such as at the most 12 consecutive amino acid residues, such as at the most 11 consecutive amino acid residues, such as at the most 10 consecutive amino acid residues, such as at the most 9 consecutive amino acid residues, such as 8 consecutive amino acid residues of SEQ ID NO: 2 or a functional homologue thereof; the functional homologue being a polypeptide of identical sequence except that at the most three amino acids have been substituted, such as at the most two amino acids have been substituted, such as at the most one amino acid has been substituted.

In another embodiment, the immunogenically active exon 9 mutant CALR peptide comprises at least 25 consecutive amino acid residues from the CALR peptide fragment of SEQ ID NO: 2, such as at least 26 consecutive amino acid residues, such as at least 27 consecutive amino acid residues, such as at least 28 consecutive amino acid residues, such 29 consecutive amino acid residues, from the exon 9 mutant CALR peptide fragment as identified in SEQ ID NO: 2 or a functional homologue thereof; the functional homologue being a polypeptide of identical sequence except that at the most three amino acids have been substituted, such as at the most two amino acids have been substituted, such as at the most one amino acid has been substituted.

In another embodiment of the invention, the immunogenically active exon 9 mutant CALR peptide comprises at the most 15 consecutive amino acid residues from SEQ ID NO: 3, such as at the most 12 consecutive amino acid residues, such as at the most 11 consecutive amino acid residues, such as at the most 10 consecutive amino acid residues, such as at the most 9 consecutive amino acid residues, such as 8 consecutive amino acid residues SEQ ID NO: 3 or a functional homologue thereof; the functional homologue being a polypeptide of identical sequence except that at the most three amino acids have been substituted, such as at the most two amino acids have been substituted, such as at the most one amino acid has been substituted.

In another embodiment, the immunogenically active exon 9 mutant CALR peptide comprises at least 25 consecutive amino acid residues from the CALR peptide fragment of SEQ ID NO: 3, such as at least 26 consecutive amino acid residues, such as at least 27 consecutive amino acid residues, such as at least 28 consecutive amino acid residues, such 29 consecutive amino acid residues, from the exon 9 mutant CALR peptide fragment as identified in SEQ ID NO: 3 or a functional homologue thereof; the functional homologue being a polypeptide of identical sequence except that at the most three amino acids have been substituted, such as at the most two amino acids have been substituted, such as at the most one amino acid has been substituted.

In another embodiment of the invention, the immunogenically active exon 9 mutant CALR peptide comprises at the most 15 consecutive amino acid residues from the CALR peptide fragment of SEQ ID NO: 15, such as at the most 12 consecutive amino acid residues, such as at the most 11 consecutive amino acid residues, such as at the most 10 consecutive amino acid residues, such as at the most 9 consecutive amino acid residues, such as 8 consecutive amino acid residues, from the exon 9 mutant CALR peptide fragment as identified in SEQ ID NO: 15 or a functional homologue thereof; the functional homologue being a polypeptide of identical sequence except that at the most three amino acids have been substituted, such as at the most two amino acids have been substituted, such as at the most one amino acid has been substituted.

In another embodiment of the invention, the immunogenically active exon 9 mutant CALR peptide comprises at least 15 consecutive amino acid residues from the CALR peptide fragment of SEQ ID NO: 15, such as at least 16 consecutive amino acid residues, such as at least 17 consecutive amino acid residues, such as a at least 18 consecutive amino acid residues, such as at least 19 consecutive amino acid residues, such at 20 consecutive amino acid residues, from the exon 9 mutant CALR peptide fragment as identified in SEQ ID NO: 15 or a functional homologue thereof; the functional homologue being a polypeptide of identical sequence except that at the most three amino acids have been substituted, such as at the most two amino acids have been substituted, such as at the most one amino acid has been substituted.

In another embodiment of the invention, the immunogenically active exon 9 mutant CALR peptide comprises at the most 30 consecutive amino acid residues from the CALR peptide fragment of SEQ ID NO: 1, such as at the most 29 consecutive amino acid residues, such as at the most 30 consecutive amino acid residues, such as at the most 31 consecutive amino acid residues, such as at the most 32 consecutive amino acid residues, such as 33 consecutive amino acid residues, from the exon 9 mutant CALR peptide fragment as identified in SEQ ID NO: 1 or a functional homologue thereof; the functional homologue being a polypeptide of identical sequence except that at the most three amino acids have been substituted, such as at the most two amino acids have been substituted, such as at the most one amino acid has been substituted.

In another embodiment of the invention, the immunogenically active exon 9 mutant CALR peptide comprises at least 32 consecutive amino acid residues from the CALR peptide fragment of SEQ ID NO: 1, such as at least 33 consecutive amino acid residues, such as at least 34 consecutive amino acid residues, such as a at least 35 consecutive amino acid residues, such as 36 consecutive amino acid residues, from the exon 9 mutant CALR peptide fragment as identified in SEQ ID NO: 1 or a functional homologue thereof; the functional homologue being a polypeptide of identical sequence except that at the most three amino acids have been substituted, such as at the most two amino acids have been substituted, such as at the most one amino acid has been substituted.

In another embodiment of the invention, the immunogenically active exon 9 mutant CALR peptide comprises at the most 35 consecutive amino acid residues from the CALR peptide fragment of SEQ ID NO: 17, such as at the most 36 consecutive amino acid residues, such as at the most 37 consecutive amino acid residues, such as at the most 38 consecutive amino acid residues, such as at the most 39 consecutive amino acid residues, such as 40 consecutive amino acid residues, from the exon 9 mutant CALR peptide fragment as identified in SEQ ID NO: 17 or a functional homologue thereof; the functional homologue being a polypeptide of identical sequence except that at the most three amino acids have been substituted, such as at the most two amino acids have been substituted, such as at the most one amino acid has been substituted.

In another embodiment of the invention, the immunogenically active exon 9 mutant CALR peptide comprises at least 39 consecutive amino acid residues from the CALR peptide fragment of SEQ ID NO: 17, such as at least 40 consecutive amino acid residues, such as at least 41 consecutive amino acid residues, such as a at least 42 consecutive amino acid residues, such as at least 43 consecutive amino acid residues, such at 44 consecutive amino acid residues, from the exon 9 mutant CALR peptide fragment as identified in SEQ ID NO: 17 or a functional homologue thereof; the functional homologue being a polypeptide of identical sequence except that at the most three amino acids have been substituted, such as at the most two amino acids have been substituted, such as at the most one amino acid has been substituted.

In some embodiments of the invention the immunogenically active exon 9 mutant CALR peptide may be selected from the group consisting of peptides of SEQ ID NO: 16, SEQ ID NO: 2, SEQ ID NO: 3, SEQ ID NO: 15, SEQ ID NO: 1 or SEQ ID NO: 17 or a functional homologue thereof. In a particular embodiment, the peptide is as set forth in SEQ ID NO: 16 or a functional homologue thereof. In another embodiment, the peptide is as set forth in SEQ ID NO: 2 or a functional homologue thereof. In another embodiment, the peptide is as set forth in SEQ ID NO: 3 or a functional homologue thereof. In another embodiment, the peptide is as set forth in SEQ ID NO: 15 or a functional homologue thereof. In another embodiment, the peptide is as set forth in SEQ ID NO: 1 or a functional homologue thereof. In another embodiment, the peptide is as set forth in SEQ ID NO: 17 or a functional homologue thereof. The functional homologue may be a polypeptide of at least 70% sequence identity with the peptides of SEQ ID NO: 16, SEQ ID NO: 2, SEQ ID NO: 3, SEQ ID NO: 15, SEQ ID NO: 1 or SEQ ID NO: 17, such as at least 75% sequence identity, such as at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity therewith. Preferably, the percentage of sequence identity between a sequence and SEQ ID NO: 16, SEQ ID NO: 2, SEQ ID NO: 3, SEQ ID NO: 15, SEQ ID NO: 1 or SEQ ID NO: 17 is determined by measuring sequence identity across the full length of SEQ ID NO: 16, SEQ ID NO: 2, SEQ ID NO: 3, SEQ ID NO: 15, SEQ ID NO: 1 or SEQ ID NO: 17.

Accordingly, in a preferred embodiment, the immunogenically active exon 9 mutant CALR peptide may be the CALR peptide fragment of SEQ ID NO: 16 or a functional homologue thereof, the functional homologue being a polypeptide of at least 70% sequence identity therewith, such as at least 75% sequence identity, such as at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity therewith.

In another preferred embodiment, the immunogenically active peptide may be the CALR peptide fragment of SEQ ID NO: 2 or a functional homologue thereof, the functional homologue being a polypeptide of at least 70% sequence identity therewith, such as at least 75% sequence identity, such as at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity therewith.

In another preferred embodiment, the immunogenically active CALR peptide may be the CALR peptide fragment of SEQ ID NO: 3 or a functional homologue thereof, the functional homologue being a polypeptide of at least 70% sequence identity therewith, such as at least 75% sequence identity, such as at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity therewith.

Accordingly, in a preferred embodiment, the immunogenically active exon 9 mutant CALR peptide may be the CALR peptide fragment of SEQ ID NO: 15 or a functional homologue thereof, the functional homologue being a polypeptide of at least 70% sequence identity therewith, such as at least 75% sequence identity, such as at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity therewith.

In another preferred embodiment, the immunogenically active peptide may be the CALR peptide fragment of SEQ ID NO: 1 or a functional homologue thereof, the functional homologue being a polypeptide of at least 70% sequence identity therewith, such as at least 75% sequence identity, such as at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity therewith.

In another preferred embodiment, the immunogenically active CALR peptide may be the CALR peptide fragment of SEQ ID NO: 17 or a functional homologue thereof, the functional homologue being a polypeptide of at least 70% sequence identity therewith, such as at least 75% sequence identity, such as at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity therewith.

In a preferred embodiment of the invention the immunogenically active peptide is selected from the group consisting of:
a) SEQ ID NO: 16 (CALR₃₇₈₋₄₁₁);
b) SEQ ID NO: 2 (CALR₃₆₇₋₃₉₆);
c) SEQ ID NO: 3 (CALR₃₈₃₋₄″); and
d) a functional homologue of the polypeptide according to any of a) to c); the functional homologue being a polypeptide of identical sequence except that at the most three amino acids have been substituted, such as at the most two amino acids have been substituted, such as at the most one amino acid has been substituted.

In a preferred embodiment of the invention the immunogenically active peptide is selected from the group consisting of:
a) SEQ ID NO: 16 (CALR₃₇₈₋₄₁₁),
b) SEQ ID NO: 2 (CALR₃₆₇₋₃₉₆);
c) SEQ ID NO: 3 (CALR₃₈₃₋₄₁₁);
d) SEQ ID NO: 15 (CALR₃₇₃₋₃₉₃);
e) SEQ ID NO: 1 (CALR₃₇₆₋₄₁₁);
f) SEQ ID NO: 17 (CALR₃₆₇₋₄₁₁); and
g) a functional homologue of the polypeptide according to any of a) to f); the functional homologue being a polypeptide of identical sequence except that at the most three amino acids have been substituted, such as at the most two amino acids have been substituted, such as at the most one amino acid has been substituted.

In some embodiments the immunogenically active peptide consists of up to 50, such as up to 40, such as up to 30, such as up to 25, such as up to 20, amino acids and comprises a sequence selected from the group consisting of:
a) SEQ ID NO: 16 (CALR₃₇₈₋₄₁₁);
b) SEQ ID NO: 2 (CALR₃₆₇₋₃₉₆);
c) SEQ ID NO: 3 (CALR₃₈₃₋₄₁₁);
d) SEQ ID NO: 15 (CALR₃₇₃₋₃₉₃);
e) SEQ ID NO: 1 (CALR₃₇₆₋₄₁₁);
f) SEQ ID NO: 17 (CALR₃₆₇₋₄₁₁); and
g) a functional homologue of the polypeptide according to any of a) to f); the functional homologue being a polypeptide of identical sequence except that at the most three amino acids have been substituted, such as at the most two amino acids have been substituted, such as at the most one amino acid has been substituted.

The up to 50, 40, 30, 25 or 20 amino acids of the said peptide may preferably consist of a sequence of consecutive amino acids of a CalR protein, within which consecutive amino acids the sequence selected from any one of (a) to (g) is comprised.

Without being bound by theory, in some cases stability of the peptides may be increased by the incorporation of additional terminal residues, at the N terminus, at the C terminus, or at both termini, for example hydrophilic amino acid residues.

Other CALR peptides of the invention comprise (or more preferably consist of) between 8 and 90, preferably between 8 and 80, more preferably between 8 and 70, yet more preferably between 8 and 60, even more preferably between 8 and 40, such as between 18 and 25 contiguous amino acids of the exon 9 mutant CALR of SEQ ID NO: 10 or a functional homologue thereof having at least 70%, preferably at least 80%, more preferably at least 90%, even more preferably at least 95%, yet more preferably at least 98%, for example at least 99% sequence identity to SEQ ID NO: 10.

In a specific embodiment the immunogenically active JAK2V617F peptide fragment of the invention consists of at the most 90 consecutive amino acid residues, such as at the most 80 consecutive amino acids residues, for example at the most 70 consecutive amino acid residues, such as at the most 60 consecutive amino acid residues, for example at the most 50 consecutive amino acid residues, for example at the most 45 consecutive amino acid residues, such as at the most 40 consecutive amino acid residues, for example at the most 35 consecutive amino acid residues, such as at the most 30 consecutive amino acid residues, for example at the most 29 consecutive amino acid residues, such as at the most 24 consecutive amino acid residues, such as at the most 22 consecutive amino acid residues, such as at the most 20 consecutive amino acid residues, such as at the most 15 consecutive amino acid residues, such as at the most 10 consecutive amino acid residues, such as at the most 9 consecutive amino acid residues, such as at the most 8 consecutive amino acid residues of JAK2V617F as identified in SEQ ID NO: 6 or a functional homologue thereof; the functional homologue being a polypeptide of identical sequence except that at the most three amino acids have been substituted, such as at the most two amino acids have been substituted, such as at the most one amino acid has been substituted. The substitution may be a conservative substitution.

Said immunogenically active peptide JAK2V617F fragment may also consist of at the most 80 consecutive amino acids residues, for example at the most 70 consecutive amino acid residues, such as at the most 60 consecutive amino acid residues, for example at the most 50 consecutive amino acid residues, for example at the most 45 consecutive amino acid residues, such as at the most 40 consecutive amino acid residues, for example at the most 35 consecutive amino acid residues, such as at the most 30 consecutive amino acid residues, for example at the most 29 consecutive amino acid residues, such as at the most 24 consecutive amino acid residues, such as at the most 22 consecutive amino acid residues, such as at the most 20 consecutive amino acid residues of CALR as identified in SEQ ID NO: 6, such as 15 to 25 consecutive amino acid residues, such as 8 to 10 consecutive amino acid residues of JAK2V617F as identified in SEQ ID NO: 6, wherein one or more amino acids have been mutated to another amino acid or deleted.

Said immunogenically active peptide JAK2V617F fragment may also consist of at least 5 consecutive amino acids residues, for example at least 6 consecutive amino acid residues, such as at least 7 consecutive amino acid residues, for at least 8 consecutive amino acid residues, for example at least 9 consecutive amino acid residues, such as at least 10 consecutive amino acid residues, for example at least 15 consecutive amino acid residues, such as at least 20 consecutive amino acid residues, for example at least 25 consecutive amino acid residues, such as at least 30 consecutive amino acid residues, such as at least 35 consecutive amino acid residues, such as at least 40 consecutive amino acid residues of JAK2 as identified in SEQ ID NO: 6, such as 8 to 15 consecutive amino acid residues, such as 9 to 10 consecutive amino acid residues of JAK2V617F as identified in SEQ ID NO: 6, wherein one or more amino acids have been substituted, mutated to another amino acid or deleted.

In one preferred embodiment of the invention, the immunogenically active peptide fragment consists of in the range of 8 to 9 amino acids, preferably of 9 consecutive amino acids of JAK2V617F as identified in SEQ ID NO: 7 or a functional homologue thereof; the functional homologue being a polypeptide of identical sequence except that at the most three amino acids have been substituted, such as at the most two amino acids have been substituted, such as at the most one amino acid has been substituted.

Accordingly in another embodiment the immunogenically active JAK2V617F peptide fragment of the invention consists of at the most 35 amino acid residues which may be consecutive, such as at the most 34 amino acid residues, for example at the most 33 amino acid residues, such as at the most 32 amino acid residues, for example at the most 31 amino acid residues, such as at the most 30 amino acid residues, for example at the most 29 amino acid residues, such as at the most 28 amino acid residues, for example at the most 27 amino acid residues, such as at the most 26 amino acid residues, for example at the most 25 amino acid residues, such as at the most 24 amino acid residues, for example at the most 23 amino acid residues, such as at the most 22 amino acid residues, for example at the most 21 amino acid residues, such as at the most 20 amino acid residues, for example at the most 19 amino acid residues, such as at the most 18 amino acid residues, such as at the most 17 amino acid residues, for example at the most 16 amino acid residues, such as at the most 15 amino acid residues, for example at the most 14 amino acid residues, such as at the most 13 amino acid residues, for example at the most 12 amino acid residues, such as at the most 11 amino acid residues, such as 8 to 10 amino acid residues, such as 9 to 10 amino acids residues from JAK2V617F of SEQ ID NO: 6 or a functional homologue thereof; the functional homologue being a polypeptide of identical sequence except that at the most three amino acids have been substituted, such as at the most two amino acids have been substituted, such as at the most one amino acid has been substituted.

In another embodiment the immunogenically active JAK2V617F peptide fragment of the invention consists of at the most 35 amino acid residues which may be consecutive, such as at the most 34 amino acid residues, for example at the most 33 amino acid residues, such as at the most 32 amino acid residues, for example at the most 31 amino acid residues, such as at the most 30 amino acid residues, for example at the most 29 amino acid residues, such as at the most 28 amino acid residues, for example at the most 27 amino acid residues, such as at the most 26 amino acid residues, for example at the most 25 amino acid residues, such as at the most 24 amino acid residues, for example at the most 23 amino acid residues, such as at the most 22 amino acid residues, for example at the most 21 amino acid residues, such as at the most 20 amino acid residues, for example at the most 19 amino acid residues, such as at the most 18 amino acid residues, such as at the most 17 amino acid residues, for example at the most 16 amino acid residues, such as at the most 15 amino acid residues, for example at the most 14 amino acid residues, such as at the most 13 amino acid residues, for example at the most 12 amino acid residues, such as at the most 11 amino acid residues, such as 8 to 10 amino acid residues, such as 9 to 10 amino acids residues from JAK2V617F of SEQ ID NO: 6 or a functional homologue thereof; the functional homologue being a polypeptide of identical sequence except that at the most three amino acids have been substituted, such as at the most two amino acids have been substituted, such as at the most one amino acid has been substituted.

In another embodiment the immunogenically JAK2V617F peptide fragment of the invention consists of at least 8 amino acid residues, such as at least 9 amino acid residues, for example at least 10 amino acid residues, such as at least 11 amino acid residues, for example at least 12 amino acid residues, such as at least 13 amino acid residues, for example at least 14 amino acid residues, such as at least 15 amino acid residues, for example at least 16 amino acid residues, such as at least 17 amino acid residues, for example at least 18 amino acid residues, such as at least 19 amino acid residues, for example at least 20 amino acid residues, such as at least 21 amino acid residues, for example at least 22 amino acid residues, such as at least 23 amino acid residues, for example at least 24 amino acid residues, such as at least 25 amino acid residues, for example at least 26 amino acid residues, such as at least 27 amino acid residues, for example at least 28 amino acid residues, such as at least 29 amino acid residues, such as 25 to 35 amino acid residues, such as 26 to 32 consecutive amino acids residues from SEQ ID NO: 6 or a functional homologue thereof; the functional homologue being a polypeptide of identical sequence except that at the most three amino acids have been substituted, such as at the most two amino acids have been substituted, such as at the most one amino acid has been substituted.

In one preferred embodiment of the invention, the immunogenically active JAK2V617F peptide comprises at the most 29 consecutive amino acid residues from JAK2V617F, such as at the most 28 consecutive amino acid residues, such as 27 consecutive amino acid residues, such as 26 consecutive amino acid residues from SEQ ID NO: 6 comprising SEQ ID NO: 7 or a functional homologue thereof; the functional homologue being a polypeptide of identical sequence except that at the most three amino acids have been substituted, such as at the most two amino acids have been substituted, such as at the most one amino acid has been substituted.

In another embodiment of the invention, the immunogenically active JAK2V617F peptide comprises at least 5 consecutive amino acid residues from the JAK2V617F peptide fragment of SEQ ID NO: 7, such as at least 6 consecutive amino acid residues, such as at least 7 consecutive amino acid residues, such as a at least 8 consecutive amino acid residues, such as 9 consecutive amino acid residues, from the JAK2V617F peptide fragment as identified in SEQ ID NO: 7 or a functional homologue thereof; the functional homologue being a polypeptide of identical sequence except that at the most three amino acids have been substituted, such as at the most two amino acids have been substituted, such as at the most one amino acid has been substituted.

In particular, the immunogenically active JAK2V617F peptide may consist of in the range of 8 to 29 consecutive amino acid residues from the JAK2V617F of SEQ ID NO: 6. In some embodiments, the immunogenically peptide comprises of consists of at the most 29 consecutive amino acid residues from JAK2V617F, such as at the most 28 consecutive amino acid residues, such as 27 consecutive amino acid residues, such as 26 consecutive amino acid residues of JAK2V617F of SEQ ID NO: 6 or a functional homologue thereof; the functional homologue being a polypeptide of identical sequence except that at the most three amino acids have been substituted, such as at the most two amino acids have been substituted, such as at the most one amino acid has been substituted. In particular, the immunogenically active peptide may consist of 29 consecutive amino acid residues from the JAK2V617F mutant of SEQ ID NO: 6 or the immunogenically active peptide may consist of 28 consecutive amino acid residues from the JAK2V617F of SEQ ID NO: 6.

In another embodiment, the immunogenically active JAK2V617F peptide may consist of in the range of 8 to 29 consecutive amino acid residues from the JAK2V617F mutant of SEQ ID NO: 6 or the immunogenically active peptide may consist of in the range of 25 to 29 consecutive amino acid residues from the JAK2V617F mutant of SEQ ID NO: 6. In some embodiments, the immunogenically peptide comprises at the most 29 consecutive amino acid residues from a JAK2V617F mutant, such as at the most 28 consecutive amino acid residues, such as 27 consecutive amino acid residues, such as 26 consecutive amino acid residues from a fragment of an JAK2V617F mutant as identified in SEQ ID NO: 6 or a functional homologue thereof; the functional homologue being a polypeptide of identical sequence except that at the most three amino acids have been substituted, such as at the most two amino acids have been substituted, such as at the most one amino acid has been substituted. In particular, the immunogenically active peptide may consist of 29 consecutive amino acid residues from the JAK2V617F mutant of SEQ ID NO: 6 or the immunogenically active peptide may consist of 28 consecutive amino acid residues from the JAK2V617F mutant of SEQ ID NO: 6.

In particular, the immunogenically active JAK2V617F peptide may consist of 9 consecutive amino acid residues from the JAK2V617F mutant of SEQ ID NO: 6 or the immunogenically active peptide may consist of up to 30 consecutive amino acid residues from JAK2V617F of SEQ ID NO: 6. In some embodiments, the immunogenically peptide comprises at the most 30 consecutive amino acid residues from JAK2V617F, such as at the most 29 consecutive amino acid residues, such as 28 consecutive amino acid residues, such as 27 consecutive amino acid residues, such as 26 consecutive amino acid residues, such as 25 consecutive amino acid residues of JAK2V617F identified in SEQ ID NO: 6 or a functional homologue thereof; the functional homologue being a polypeptide of identical sequence except that at the most three amino acids have been substituted, such as at the most two amino acids have been substituted, such as at the most one amino acid has been substituted. In particular, the immunogenically active peptide may consist of 9 consecutive amino acid residues from the JAK2V617F fragment of SEQ ID NO: 7 or the immunogenically active peptide may consist of 8 consecutive amino acid residues from the JAK2V617F fragment of SEQ ID NO: 7.

In another embodiment of the invention, the immunogenically active JAK2V617F peptide comprises at the most 9 consecutive amino acid residues from the JAK2V617F peptide fragment of SEQ ID NO: 7, such as at the most 8 consecutive amino acid residues, such as at the most 7 consecutive amino acid residues, such as at the most 6 consecutive amino acid residues, such as at the most 5 consecutive amino acid residues from the JAK2V617F peptide fragment as identified in SEQ ID NO: 7 or a functional homologue thereof; the functional homologue being a polypeptide of identical sequence except that at the most three amino acids have been substituted, such as at the most two amino acids have been substituted, such as at the most one amino acid has been substituted.

In another embodiment the immunogenically active JAK2V617F peptide fragment of the invention consists of at least 5 amino acid residues, such as at least 6 amino acid residues, for example at least 7 amino acid residues, such as at least 8 amino acid residues, for example 9 consecutive amino acids residues from SEQ ID NO: 7 or a functional homologue thereof; the functional homologue being a polypeptide of identical sequence except that at the most three amino acids have been substituted, such as at the most two amino acids have been substituted, such as at the most one amino acid has been substituted.

In some embodiments of the invention the immunogenically active JAK2V617F peptide may comprise or consist of SEQ ID NO: 7 or a functional homologue thereof. In a particular embodiment, the peptide is as set forth in SEQ ID NO: 7 or a functional homologue thereof. The functional homologue may be a polypeptide of at least 70% sequence identity with the peptides of SEQ ID NO: 7, such as at least 75% sequence identity, such as at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity therewith. Preferably, the percentage of sequence identity between a sequence and SEQ ID NO: 7 is determined by measuring sequence identity across the full length of SEQ ID NO: 7.

Accordingly, in a preferred embodiment, the immunogenically active JAK2V617F peptide may be the JAK2V617F peptide fragment of SEQ ID NO: 7 or a functional homologue thereof, the functional homologue being a polypeptide of at least 70% sequence identity therewith, such as at least 75% sequence identity, such as at least 80% sequence identity, such as at least 85% sequence identity, such as at least 90% sequence identity, such as at least 95% sequence identity, such as at least 96% sequence identity, such as at least 97% sequence identity, such as at least 98% sequence identity, such as at least 99% sequence identity therewith.

In a preferred embodiment of the invention the immunogenically active peptide is selected from the group consisting of:
a) SEQ ID NO: 7 (JAK2₆₁₀₋₆₁₈) or a peptide comprising SEQ ID NO: 7 as defined herein; and
b) a functional homologue of the polypeptide according to a); the functional homologue being a polypeptide of identical sequence except that at the most three amino acids have been substituted, such as at the most two amino acids have been substituted, such as at the most one amino acid has been substituted.

The peptide may consist of up to 50, 40, 30, 25 or 20 consecutive amino acids of a JAK2 protein, within which consecutive amino acids the sequence selected from any one of (a) or (b) is comprised.

Without being bound by theory, in some cases stability of the peptides may be increased by the incorporation of additional terminal residues, at the N terminus, at the C terminus, or at both termini, for example hydrophilic amino acid residues.

Other JAK2V617F peptides of the invention comprise (or more preferably consist of) between 8 and 90, preferably between 8 and 80, more preferably between 8 and 70, yet more preferably between 8 and 60, even more preferably between 8 and 40, such as between 18 and 25 contiguous amino acids of the JAK2V617F mutant of SEQ ID NO: 6 or a functional homologue thereof having at least 70%, preferably at least 80%, more preferably at least 90%, even more preferably at least 95%, yet more preferably at least 98%, for example at least 99% sequence identity to SEQ ID NO: 6.

TABLE 1
Exemplary exon 9 mutant CALR and JAK2V617F
peptides

| | | Amino | |
|---|---|---|---|
| | | acid | |
| | | numbers | |
| SEQ | | in | |
| ID | | parent | |
| NO | Name | sequence | Sequence |
| 1 | CALR₃₇₈₋₄₁₁ | 378-411 | RRMRRTRRKMRRKMSPARPRTSCRE |
| | | | ACLQGWTE |
| 2 | CALR₃₆₇₋₃₉₆ | 367-396 | RRMMRTKMRMRRMRRTRRKMRRKMS |
| | | | PARP |
| 3 | CALR₃₈₃₋₄₁₁ | 383-411 | TRRKMRRKMSPARPRTSCREACLQG |
| | | | WTEA |
| 7 | JAK2₆₁₀₋₆₁₈ | 610-618 | VLNYGVCFC |
| 14 | CALR₃₆₁₋₄₁₁ | 361-411 | RRMMRTKMRMRRMRRTRRKMRRKMS |
| | | | PARPRTSCREACLQGWTE |
| 15 | CALR₃₇₃₋₃₉₃ | 373-393 | KMRMRRMRRTRRKMRRKMSP |
| 16 | CALR₃₇₅₋₄₁₁ | 375-411 | RMRRMRRTRRKMRRKMSPARPRTSC |
| | | | REACLQGWTEA |
| 17 | CALR₃₆₇₋₄₁₁ | 367-411 | RRMMRTKMRMRRMRRTRRKMRRKMS |
| | | | PARPRTSCREACLQGWTEA |

Functional Homologues

Functional homologues of exon 9 CALR and JAK2V617F, or immunogenically active fragments thereof, are polypeptides, which also are immunogenically active, and which share at least some degree of sequence identity with exon 9 CALR and JAK2V617F respectively, and in particular with exon 9 mutant CALR of SEQ ID NO: 10 or JAK2V617F of SEQ ID NO: 6. Functional homologues of exon 9 CALR and JAK2V617F, or immunogenically active fragments thereof, may also be polypeptides, which also are immunogenically active, and which share at least some degree of sequence identity with fragments of exon 9 mutant CALR or JAK2V617F, respectively. In particular, the exon 9 mutant CALR functional homologue may share at least some degree of sequence identity with the fragment as set forth in SEQ ID NO: 16. The JAK2V617F functional homologue may share at least some degree of sequence identity with the fragment as set forth in SEQ ID NO: 7. Functional homologues of exon 9 mutant CALR, or immunogenically active fragments thereof, may also be polypeptides, which also are immunogenically active, and which share at least some degree of sequence identity with fragments of exon 9 mutant CALR, in particular with the fragment as set forth in SEQ ID NO: 2. Functional homologues of exon 9 mutant CALR, or immunogenically active fragments thereof, may also be polypeptides, which also are immunogenically active, and which share at least some degree of sequence identity with fragments of exon 9 mutant CALR, in particular with the fragment as set forth in SEQ ID NO: 3.

For shorter polypeptides, such as for polypeptide shorter than 50 amino acids, for example shorter than 25 amino acids, a functional homologue may be an immunogenically active polypeptide of identical sequence except that at the most three amino acids have been substituted, such as at the most two amino acids have been substituted, such as at the most one amino acid has been substituted.

Alternatively, a functional homologue may be an immunogenically active exon 9 mutant CALR polypeptide sharing at least 70% sequence identity to the exon 9 mutant CALR fragment of SEQ ID NO: 16, and accordingly, a functional homologue preferably has at least 75% sequence identity, for example at least 80% sequence identity, such as at least 85% sequence identity, for example at least 89% sequence identity, such as at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human exon 9 mutant CALR of SEQ ID NO: 16.

A functional homologue may be an immunogenically active exon 9 mutant CALR polypeptide sharing at least 70% sequence identity to the exon 9 mutant CALR fragment of SEQ ID NO: 2, and accordingly, a functional homologue preferably has at least 75% sequence identity, for example at least 80% sequence identity, such as at least 85% sequence identity, for example at least 89% sequence identity, such as at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human CALR of SEQ ID NO: 2.

A functional homologue may be an immunogenically active exon 9 mutant CALR polypeptide sharing at least 70% sequence identity to the exon 9 mutant CALR fragment of SEQ ID NO: 3, and accordingly, a functional homologue preferably has at least 75% sequence identity, for example at least 80% sequence identity, such as at least 85% sequence identity, for example at least 89% sequence identity, such as at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human exon 9 mutant CALR of SEQ ID NO: 3.

A functional homologue may be an immunogenically active exon 9 mutant CALR polypeptide sharing at least 70% sequence identity to the exon 9 mutant CALR fragment of SEQ ID NO: 15, and accordingly, a functional homologue preferably has at least 75% sequence identity, for example at least 80% sequence identity, such as at least 85% sequence identity, for example at least 89% sequence identity, such as at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human exon 9 mutant CALR of SEQ ID NO: 15.

A functional homologue may be an immunogenically active exon 9 mutant CALR polypeptide sharing at least 70% sequence identity to the exon 9 mutant CALR fragment of SEQ ID NO: 1, and accordingly, a functional homologue preferably has at least 75% sequence identity, for example at least 80% sequence identity, such as at least 85% sequence identity, for example at least 89% sequence identity, such as at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human exon 9 mutant CALR of SEQ ID NO: 1.

A functional homologue may be an immunogenically active exon 9 mutant CALR polypeptide sharing at least 70% sequence identity to the exon 9 mutant CALR fragment of SEQ ID NO: 17, and accordingly, a functional homologue preferably has at least 75% sequence identity, for example at least 80% sequence identity, such as at least 85% sequence identity, for example at least 89% sequence identity, such as at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human exon 9 mutant CALR of SEQ ID NO: 17.

Alternatively, a functional homologue may be an immunogenically active polypeptide sharing at least 70% sequence identity to the JAK2V617F fragment of SEQ ID NO: 7, and accordingly, a functional homologue preferably has at least 75% sequence identity, for example at least 80% sequence identity, such as at least 85% sequence identity, for example at least 89% sequence identity, such as at least 90% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human JAK2V617F of SEQ ID NO: 7.

Preferably, the JAK2V617F fragment comprises at least amino acid 617 of SEQ ID NO: 6.

Sequence identity can be calculated using a number of well-known algorithms and applying a number of different gap penalties. The sequence identity is calculated relative to full-length reference sequence, e.g. to full length SEQ ID NO: 16. Any sequence alignment tool, such as but not limited to FASTA, BLAST, or LALIGN may be used for searching homologues and calculating sequence identity. Moreover, when appropriate any commonly known substitution matrix, such as but not limited to PAM, BLOSSUM or PSSM matrices may be applied with the search algorithm. For example, a PSSM (position specific scoring matrix) may be applied via the PSI-BLAST program. Moreover, sequence alignments may be performed using a range of penalties for gap opening and extension. For example, the BLAST algorithm may be used with a gap opening penalty in the range 5-12, and a gap extension penalty in the range 1-2.

Functional homologues may further comprise chemical modifications such as ubiquitination, labeling (e.g., with radionuclides, various enzymes, etc.), pegylation (derivatization with polyethylene glycol), or by insertion (or substitution by chemical synthesis) of amino acids (amino acids) such as ornithine, which do not normally occur in human proteins, however it is preferred that the functional equivalent does not contain chemical modifications.

Any changes made to the sequence of amino acid residues compared to that of exon 9 mutant CALR of SEQ ID NO: 10, or compared to the exon 9 mutant CALR fragments of SEQ ID NO: 16, SEQ ID NO: 2, SEQ ID NO: 3, SEQ ID NO: 15, SEQ ID NO: 1 or SEQ ID NO: 17, or compared to that of JAK2V617F of SEQ ID NO: 6, or compared to the JAK2V617F fragment of SEQ ID NO: 7, are preferably conservative substitutions. A person skilled in the art will know how to make and assess ‘conservative’ amino acid substitutions, by which one amino acid is substituted for another with one or more shared chemical and/or physical characteristics. Conservative amino acid substitutions are less likely to affect the functionality of the protein. Amino acids may be grouped according to shared characteristics. A conservative amino acid substitution is a substitution of one amino acid within a predetermined group of amino acids for another amino acid within the same group, wherein the amino acids within a predetermined groups exhibit similar or substantially similar characteristics.

Thus, in an embodiment of the present invention, the vaccine composition comprises a polypeptide consisting of a consecutive sequence of the exon 9 mutant CALR fragment of SEQ ID NO: 10 in the range of 8 to 35 amino acids, preferably in the range of 25 to 31, or 27 to 30 amino acids, wherein at the most three amino acids have been substituted, and where the substitution preferably is conservative.

Thus, in an embodiment of the present invention, the vaccine composition comprises a polypeptide consisting of a consecutive sequence of the exon 9 mutant CALR fragment of SEQ ID NO: 16 in the range of 8 to 34 amino acids, preferably in the range of 25 to 31, or 27 to 30 amino acids, wherein at the most three amino acids have been substituted, and where the substitution preferably is conservative.

In another embodiment of the present invention, the vaccine composition comprises a polypeptide consisting of a consecutive sequence of the exon 9 mutant CALR fragment of SEQ ID NO: 2 in the range of 8 to 29 amino acids, preferably in the range of 20 to 29, or 27 to 29 amino acids, wherein at the most three amino acids have been substituted, and where the substitution preferably is conservative.

In another embodiment of the present invention, the vaccine composition comprises a polypeptide consisting of a consecutive sequence of the exon 9 mutant CALR fragment of SEQ ID NO: 3 in the range of 8 to 29 amino acids, preferably in the range of 20 to 29, or 27 to 29 amino acids, wherein at the most three amino acids have been substituted, and where the substitution preferably is conservative.

In another embodiment of the present invention, the vaccine composition comprises a polypeptide consisting of a consecutive sequence of the exon 9 mutant CALR fragment of SEQ ID NO: 15 in the range of 8 to 20 amino acids, preferably in the range of 15 to 20, or 17 to 20 amino acids, wherein at the most three amino acids have been substituted, and where the substitution preferably is conservative.

In another embodiment of the present invention, the vaccine composition comprises a polypeptide consisting of a consecutive sequence of the exon 9 mutant CALR fragment of SEQ ID NO: 1 in the range of 8 to 36 amino acids, preferably in the range of 39 to 36, or 33 to 36 amino acids, wherein at the most three amino acids have been substituted, and where the substitution preferably is conservative.

In another embodiment of the present invention, the vaccine composition comprises a polypeptide consisting of a consecutive sequence of the exon 9 mutant CALR fragment of SEQ ID NO: 17 in the range of 8 to 44 amino acids, preferably in the range of 36 to 44, or 40 to 44 amino acids, wherein at the most three amino acids have been substituted, and where the substitution preferably is conservative.

In another embodiment of the present invention, the vaccine composition comprises a polypeptide consisting of a consecutive sequence of the JAK2V617F of SEQ ID NO: 6 in the range of 8 to 15 amino acids, preferably in the range of 8 to 13, or 9 to 10 amino acids, wherein at the most three amino acids have been substituted, and where the substitution preferably is conservative.

In another embodiment of the present invention, the vaccine composition comprises a polypeptide consisting of a consecutive sequence of the JAK2V617F fragment of SEQ ID NO: 7 in the range of 8 to 9 amino acids, preferably 9 amino acids, wherein at the most three amino acids have been substituted, and where the substitution preferably is conservative.

Preferably, the JAK2V617F polypeptide comprises at least amino acid 617 of SEQ ID NO: 6.

Polypeptides Comprising Exon 9 Mutant CALR or JAK2V617F or a Fragment Thereof

It is also comprised within the invention that the vaccine compositions of the invention may comprise a polypeptide comprising exon 9 mutant CALR, JAK2V617F or a fragment thereof. Thus, the immunogenically active peptide fragment of an exon 9 mutant CALR or JAK2V617F may be a polypeptide comprising an exon 9 mutant CALR or JAK2V617F fragment, for example any of the polypeptides described herein in this section.

In particular, such polypeptides may comprise full length exon 9 mutant CALR, such as any of the exon 9 mutant CALRs described herein above in the section “Calreticulin (CALR)”. The polypeptides may comprise full length JAK2V617F, such as any of the JAK2V617F described herein above in the section “Janus kinase 2 (JAK2)”.

For example, the polypeptide may comprise exon 9 mutant CALR of SEQ ID NO: 10 or a functional homologue thereof sharing at least 70%, such as at least 80%, for example at least 90%, such as at least 95% sequence identity therewith. In particular, such polypeptides may comprise at the most 90, such as at the most 50, for example at the most 29, such as at the most 25, for example at the most 10 amino acids in addition to CALR of SEQ ID NO: 10. For example the polypeptide may comprise exon 9 mutant fragment CALR of SEQ ID NO: 16, SEQ ID NO: 2, SEQ ID NO: 3, SEQ ID NO: 15, SEQ ID NO: 1 or SEQ ID NO: 17 or a functional homologue thereof sharing at least 70%, such as at least 80%, for example at least 90%, such as at least 95% sequence identity therewith. In particular, such polypeptides may comprise at the most 90, such as at the most 50, for example at the most 25, such as at the most 10 amino acids in addition to CALR of SEQ ID NO: 10.

In some embodiments, the polypeptide may comprise the JAK2V617F mutant of SEQ ID NO: 6 or a functional homologue thereof sharing at least 70%, such as at least 80%, for example at least 90%, such as at least 95% sequence identity therewith. In particular, such polypeptides may comprise at the most 90, such as at the most 50, for example at the most 29, such as at the most 25, for example at the most 10 amino acids in addition to JAK2V617F of SEQ ID NO: 6. For example the polypeptide may comprise JAK2V617F of SEQ ID NO: 6 or a functional homologue thereof sharing at least 70%, such as at least 80%, for example at least 90%, such as at least 95% sequence identity therewith. In particular, such polypeptides may comprise at the most 90, such as at the most 50, for example at the most 25, such as at the most 10 amino acids in addition to JAK2V617F of SEQ ID NO: 6.

Preferably, the JAK2V617F polypeptide comprised in the vaccine composition comprises at least amino acid 617 of SEQ ID NO: 6.

It is also comprised within the invention that the vaccine compositions may comprise a polypeptide comprising a fragment of an exon 9 mutant CALR, such as any of the fragments described herein above in the section “Immunogenically active peptide fragment of exon 9 mutant CALR or JAK2V617F”, or a fragment of JAK2V617F, such as any of the fragments described herein above in the section “Immunogenically active peptide fragment of exon 9 mutant CALR or JAK2V617F”.

Thus, said polypeptide may be an exon 9 mutant CALR polypeptide of at the most 400 amino acids, such as at the most 300 amino acids, for example at the most 200 amino acids, such as at the most 100 amino acids, for example at the most 50 amino acids comprising a consecutive sequence of amino acids of SEQ ID NO: 16, wherein said consecutive sequence of amino acids of SEQ ID NO:1 consists of at the most 50 amino acid residues, for example at the most 45 amino acid residues, such as at the most 40 amino acid residues, for example at the most 35 amino acid residues, such as at the most 30 amino acid residues, for example at the most 25 amino acid residues, such as in the range of 18 to 25, such as in the range of 5 to 10 consecutive amino acids of SEQ ID NO: 16 or a functional homologue thereof. Said polypeptide thus be any of the following exon 9 CALR mutants: L367fs*46 (full length set forth in SEQ ID NO: 10), E370fs*43, E370fs*48, L367fs*48, L367fs*44, K368fs*51, L367fs*52, R366fs*53, E371fs*49, K368fs*43, E370fs*37, D373fs*47, K374fs*53, E371fs*49, K385fs*47, K385fs*47, R376fs*55, K385fs*47, E381fs*48 (Nangalia et al., 2013; the sequences of the above mutations are listed in FIG. 3, panel A, p. 2400 of Nangalia et al.).

In particular, said exon 9 CALR polypeptide may be a polypeptide of at the most 100 amino acid residues, such as at the most 90 amino acid residues, such as at the most 80 amino acid residues, for example at the most 70 amino acid residues, such as at the most 60 amino acid residues, for example at the most 50 amino acid residues, for example at the most 45 amino acid residues, such as at the most 40 amino acid residues, for example at the most 35 amino acid residues, such as at the most 30 amino acid residues comprising an immunogenically active peptide selected from the group consisting of:
a) SEQ ID NO: 16 (CALR₃₇₈₋₄₁₁.);
b) SEQ ID NO: 2 (CALR₃₆₇₋₃₉₆);
c) SEQ ID NO: 3 (CALR₃₈₃₋₄₁₁); and
d) a functional homologue of the polypeptide according to any of a) to c); the functional homologue being a polypeptide of identical sequence except that at the most three amino acids have been substituted, such as at the most two amino acids have been substituted, such as at the most one amino acid has been substituted.

In particular, said exon 9 CALR polypeptide may be a polypeptide of at the most 100 amino acid residues, such as at the most 90 amino acid residues, such as at the most 80 amino acid residues, for example at the most 70 amino acid residues, such as at the most 60 amino acid residues, for example at the most 50 amino acid residues, for example at the most 45 amino acid residues, such as at the most 40 amino acid residues, for example at the most 35 amino acid residues, such as at the most 30 amino acid residues comprising an immunogenically active peptide selected from the group consisting of:
a) SEQ ID NO: 16 (CALR₃₇₈₋₄₁₁);
b) SEQ ID NO: 2 (CALR₃₆₇₋₃₉₆);
c) SEQ ID NO: 3 (CALR₃₈₃₋₄₁₁);
d) SEQ ID NO: 15 (CALR₃₇₃₋₃₉₃);
e) SEQ ID NO: 1 (CALR₃₇₆₋₄₁₁);
f) SEQ ID NO: 17 (CALR₃₆₇₋₄₁₁); and
g) a functional homologue of the polypeptide according to any of a) to f); the functional homologue being a polypeptide of identical sequence except that at the most three amino acids have been substituted, such as at the most two amino acids have been substituted, such as at the most one amino acid has been substituted.

Said exon 9 mutant CALR polypeptide may also be a polypeptide of at the most 100 amino acids, such as at the most 50 amino acids, for example at the most 30 amino acids, such as at the most 20 amino acids, for example at the most 15 amino acids comprising a consecutive sequence of amino acids of SEQ ID NO: 16, wherein said consecutive sequence of amino acids of SEQ ID NO: 16 consists of in the range of 8 to 10, such as of 9 or 10 consecutive amino acids from CALR of SEQ ID NO: 16 or a functional homologue thereof. Thus, said polypeptide may be a polypeptide of at the most 100 amino acids, such as at the most 50 amino acids, for example at the most 30 amino acids, such as at the most 29 amino acids comprising an immunogenically active peptide selected from the group consisting of:
a) SEQ ID NO: 16 (CALR₃₇₈₋₄₁₁);
b) SEQ ID NO: 2 (CALR₃₆₇₋₃₉₆);
c) SEQ ID NO: 3 (CALR₃₈₃₋₄₁₁);
d) SEQ ID NO: 15 (CALR₃₇₃₋₃₉₃);
e) SEQ ID NO: 1 (CALR₃₇₆₋₄₁₁);
f) SEQ ID NO: 17 (CALR₃₆₇₋₄₁₁); and
g) a functional homologue of the polypeptide according to any of a) to f); the functional homologue being a polypeptide of identical sequence except that at the most three amino acids have been substituted, such as at the most two amino acids have been substituted, such as at the most one amino acid has been substituted.

In other embodiments, the polypeptide may be a JAK2V617F polypeptide of at the most 400 amino acids, such as at the most 300 amino acids, for example at the most 200 amino acids, such as at the most 100 amino acids, for example at the most 50 amino acids comprising a consecutive sequence of amino acids of SEQ ID NO: 6, wherein said consecutive sequence of amino acids of SEQ ID NO: 6 consists of at the most 50 amino acid residues, for example at the most 45 amino acid residues, such as at the most 40 amino acid residues, for example at the most 35 amino acid residues, such as at the most 30 amino acid residues, for example at the most 25 amino acid residues, such as in the range of 18 to 25, such as in the range of 8 to 10 consecutive amino acids from JAK2V617F of SEQ ID NO: 6 or a functional homologue thereof.

In particular, said JAK2V617F polypeptide may be a polypeptide of at the most 100 consecutive amino acid residues, such as at the most 90 consecutive amino acid residues, such as at the most 80 consecutive amino acid residues, for example at the most 70 consecutive amino acid residues, such as at the most 60 consecutive amino acid residues, for example at the most 50 consecutive amino acid residues, for example at the most 45 consecutive amino acid residues, such as at the most 40 consecutive amino acid residues, for example at the most 35 consecutive amino acid residues, such as at the most 30 consecutive amino acid residues, for example at the most 29 consecutive amino acid residues, such as at the most 25 consecutive amino acid residues, such as 18 to 25 consecutive amino acid residues, such as of 20 consecutive amino acids of JAK2V617F as identified in SEQ ID NO: 6 or a functional homologue thereof, and comprising an immunogenically active peptide selected from the group consisting of:
a) SEQ ID NO: 7 (JAK2₆₁₀₋₆₁₈); and
b) a functional homologue of the polypeptide according to any of a) to c); the functional homologue being a polypeptide of identical sequence except that at the most three amino acids have been substituted, such as at the most two amino acids have been substituted, such as at the most one amino acid has been substituted.

Said JAK2V617F polypeptide may also be a polypeptide of at the most 100 amino acids, such as at the most 50 amino acids, for example at the most 30 amino acids, such as at the most 20 amino acids, for example at the most 15 amino acids, such as at the most 10 amino acids, comprising a consecutive sequence of amino acids of SEQ ID NO:6, wherein said consecutive sequence of amino acids of SEQ ID NO: 6 consists of in the range of 8 to 10, such as of 9 or 10 consecutive amino acids from JAK2 of SEQ ID NO: 6 or a functional homologue thereof. Thus, said polypeptide may be a polypeptide of at the most 100 amino acids, such as at the most 50 amino acids, for example at the most 30 amino acids, such as at the most 29 amino acids, for example 26 amino acids, such as at the most 20 amino acids, for example at the most 15 amino acids comprising an immunogenically active peptide selected from the group consisting of:
a) SEQ ID NO: 7 (JAK2₆₁₀₋₆₁₈); and
b) a functional homologue of the polypeptide according to any of a) to c); the functional homologue being a polypeptide of identical sequence except that at the most three amino acids have been substituted, such as at the most two amino acids have been substituted, such as at the most one amino acid has been substituted.

Preferably, the JAK2V617F polypeptide comprises at least amino acid 617 of SEQ ID NO: 6.

MHC

It is comprised within the invention that the immunogenically active peptide fragments of exon 9 mutant CALR or of JAK2V617F mutant may be an MHC Class I-restricted peptide fragment or MHC Class II-restricted peptide fragment, such as any of the an MHC Class I-restricted peptide fragments or MHC Class II-restricted peptide fragments described in this section.

There are two types of MHC molecules; MHC class I molecules and MHC class II molecules. MHC class I molecules are recognized by CD8 T-cells, which are the principal effector cells of the adaptive immune response. MHC class II molecules are mainly expressed on the surface of antigen presenting cells (APCs), the most important of which appears to be the dendritic cells. APCs stimulate naïve T-cells, as well as other cells in the immune system. They stimulate both CD8 T-cells and CD4 T-cells.

In one embodiment, the invention provides immunogenically active CALR peptides (optionally comprised in larger peptides and/or in vaccine compositions as described herein), wherein said immunogenically active exon 9 mutant CALR peptides are MHC Class I-restricted peptide fragments consisting of 25-35 consecutive amino acids from CALR of SEQ ID NO: 10, such as the peptide fragment of SEQ ID NO: 16, SEQ ID NO: 2, SEQ ID NO: 3, SEQ ID NO: 15, SEQ ID NO: 1 or SEQ ID NO: 17 or a functional homologue thereof, wherein at the most two amino acids of SEQ ID NO: 10 or SEQ ID NO: 16, SEQ ID NO: 2, SEQ ID NO: 3, SEQ ID NO: 15, SEQ ID NO: 1 or SEQ ID NO: 17 have been substituted. In another embodiment, the invention provides immunogenically active JAK2V617F peptides (optionally comprised in larger peptides and/or in vaccine compositions as described herein), wherein said immunogenically active JAK2 V617F peptides are MHC Class I-restricted peptide fragments consisting of 5-15 consecutive amino acids from JAK2V617F of SEQ ID NO: 6, such as 8-9 peptides of the peptide fragment of SEQ ID NO: 7 or a functional homologue thereof, wherein at the most two amino acids of SEQ ID NO: 6 or SEQ ID NO: 7 have been substituted. Such MHC Class I-restricted peptide fragments are characterized by having at least one of several features, one of which is the ability to bind to the Class I HLA molecule to which it is restricted at an affinity as measured by the amount of the peptide that is capable of half maximal recovery of the Class I HLA molecule (C₅₀ value) which is at the most 50 μM as determined by the assembly binding assay as described herein. This assembly assay is based on stabilization of the HLA molecule after loading of peptide to the peptide transporter deficient cell line T2. Subsequently, correctly folded stable HLA heavy chains are immunoprecipitated using conformation dependent antibodies and the peptide binding is quantitated. The peptides of this embodiment comprise (or more preferably consist of) at the most 100, preferably at the most 50, more preferably at the most 25, yet more preferably at the most 20, yet even more preferably at the most 15, such as at the most 10, for example in the range of 25 to 35 or 8 to 12 consecutive amino acids of exon 9 CALR mutant of SEQ ID NO: 10, or of the fragment of SEQ ID NO: 16, SEQ ID NO: 2, SEQ ID NO: 3, SEQ ID NO: 15, SEQ ID NO: 1 or SEQ ID NO: 17, or a functional homologue thereof wherein at the most two amino acids of SEQ ID NO: 10, SEQ ID NO: 16, SEQ ID NO: 2, SEQ ID NO: 3, SEQ ID NO: 15, SEQ ID NO: 1 or SEQ ID NO: 17 have been substituted. The peptides may in some embodiments comprise (or more preferably consist of) at the most 100, preferably at the most 50, more preferably at the most 25, yet more preferably at the most 20, yet even more preferably at the most 15, such as at the most 10, for example in the range of 8 to 15 or 15 to 25 consecutive amino acids of JAK2V617F of SEQ ID NO: 6 or a functional homologue thereof wherein at the most two amino acids of SEQ ID NO: 6 have been substituted. Preferably, the JAK2V617F peptides comprise at least amino acid 617 of SEQ ID NO: 6.

The assembly binding assay provides a simple means of screening candidate peptides for their ability to bind to a given HLA allele molecule at the above affinity. In preferred embodiments, the peptide fragment of the invention in one having a C₅₀ value, which is at the most 30 μM, such as a C₅₀ value, which is at the most 20 μM including C₅₀ values of at the most 10 μM, at the most 5 μM and at the most 2 μM.

In another preferred embodiment, there are provided novel MHC Class II-restricted peptide fragments of exon 9 mutant CALR or JAK2V617F. In some embodiments, the MHC Class II-restricted peptide fragment is of exon 9 mutant CALR of SEQ ID NO: 10, such as the peptides of SEQ ID NO: 16, SEQ ID NO: 2 or SEQ ID NO: 3 or functional homologues thereof, wherein at the most two amino acids of SEQ ID NO: 10 have been substituted (also referred to herein as “peptides”), which are characterized by having at least one of several features described herein below. The peptides of this embodiment comprise (or more preferably consist of) between 4 and 93, preferably between 8 and 90, more preferably between 10 and 75, yet more preferably between 12 and 60, even more preferably between 20 and 40, such as between 25 and 35 consecutive amino acids of exon 9 mutant CALR of SEQ ID NO: 10 or a functional homologue thereof, wherein at the most two, preferably at the most one amino acids of SEQ ID NO: 10 have been substituted. In a preferred embodiment, the peptides comprise (or more preferably consist of) between 25 and 35, preferably between 26 and 33, more preferably between 27 and 32, yet more preferably between 28 and 31, even more preferably between 28 and 30, such as between 29 consecutive amino acids of the exon 9 mutant CALR of SEQ ID NO: 10. In another preferred embodiment, the peptides comprise (or more preferably consist of) between 25 and 35, preferably between 26 and 33, more preferably between 27 and 32, yet more preferably between 28 and 31, even more preferably between 28 and 30, such as between 29 consecutive amino acids of the CALR peptide fragment of SEQ ID NO: 16. In yet another preferred embodiment, the peptides comprise (or more preferably consist of) between 25 and 29, preferably between 26 and 29, more preferably between 27 and 29, such as 28 or 29 consecutive amino acids of the exon 9 mutant CALR peptide fragment of SEQ ID NO: 2. In yet another preferred embodiment, the peptides comprise (or more preferably consist of) between 25 and 29, preferably between 26 and 29, more preferably between 27 and 29, such as 28 or 29 consecutive amino acids of the exon 9 mutant CALR peptide fragment of SEQ ID NO: 3. In yet another preferred embodiment, the peptides comprise (or more preferably consist of) between 15 and 20, preferably between 16 and 20, more preferably between 17 and 20, such as 18 or 19 consecutive amino acids of the exon 9 mutant CALR peptide fragment of SEQ ID NO: 15. In yet another preferred embodiment, the peptides comprise (or more preferably consist of) between 32 and 36, preferably between 33 and 36, more preferably between 34 and 36, such as 35 or 36 consecutive amino acids of the exon 9 mutant CALR peptide fragment of SEQ ID NO: 1. In yet another preferred embodiment, the peptides comprise (or more preferably consist of) between 40 and 44, preferably between 41 and 44, more preferably between 40 and 41, such as 40, 41, 42, 43 or 44 consecutive amino acids of the exon 9 mutant CALR peptide fragment of SEQ ID NO: 17.

In other embodiments, the MHC Class II-restricted peptide fragment is of JAK2V617F of SEQ ID NO: 6, such as the peptide of SEQ ID NO: 7 or functional homologues thereof, wherein at the most two amino acids of SEQ ID NO: 6 have been substituted (also referred to herein as “peptides”), which are characterized by having at least one of several features described herein below. The peptides of this embodiment comprise (or more preferably consist of) between 4 and 93, such as between 5 and 90, for example between 6 and 75, such as between 7 and 60, for example between 8 and 40, such as between 9 and 30, for example between 10 and 20 consecutive amino acids of JAK2 of SEQ ID NO: 6 or a functional homologue thereof, wherein at the most two, preferably at the most one amino acids of SEQ ID NO: 6 have been substituted. In a preferred embodiment, the peptides comprise (or more preferably consist of) between 8 and 15, preferably between 8 and 14, more preferably between 8 and 13, yet more preferably between 8 and 12, even more preferably between 8 and 11, such as 9 amino acids of the JAK2V617F of SEQ ID NO: 6. In another preferred embodiment, the peptides comprise (or more preferably consist of) between 8 and 9, such as 9 amino acids of the JAK2V617F peptide fragment of SEQ ID NO: 7.

Thus there are provided novel MHC Class I-restricted peptide exon 9 mutant CALR fragments of 25-35 amino acids or novel MHC Class II-restricted peptide fragments of 25-35 amino acids of exon 9 mutant CALR of SEQ ID NO: 10 or a functional homologue thereof, wherein at the most two amino acids of SEQ ID NO: 10 have been substituted, which are characterized by having at least one of several features described herein below, one of which is the ability to bind to the Class I or Class II HLA molecule to which it is restricted. There are also provided novel MHC Class I-restricted peptide JAK2V617F fragments of 8-15 amino acids or novel MHC Class II-restricted peptide fragments of 8-15 amino acids of JAK2V617F of SEQ ID NO: 6 or a functional homologue thereof, wherein at the most two amino acids of SEQ ID NO: 6 have been substituted, which are characterized by having at least one of several features described herein below, one of which is the ability to bind to the Class I or Class II HLA molecule to which it is restricted.

In particular embodiments there is provided a peptide fragment, which is an MHC Class I-restricted peptide or an MHC class II-restricted peptide having at least one of the following characteristics:

(i) capable of eliciting INF-γ-producing cells in a PBL population of at least one cancer patient at a frequency of at least 1 per 10⁴ PBLs as determined by an ELISPOT assay, and/or

(ii) capable of in situ detection in a tumor tissue of CTLs that are reactive with the epitope peptide.

(iii) capable of inducing the growth of exon 9 mutant CALR specific T-cells in vitro and/or capable of inducing the growth of JAK2V617F specific T-cells in vitro.

More preferred peptides according to the present invention are peptides capable of raising a specific T-cell response as determined by an ELISPOT assay, for example as described in Example 1 herein below. Some peptides, although they do not bind MHC class I or class II with high affinity, may still give rise to a T-cell response as determined by ELISPOT. Other peptides capable of binding MHC class I or class II with high affinity also give rise to a T-cell response as determined by ELISPOT. Both kinds of peptides are preferred peptides according to the invention.

Hence, preferred peptides according to the present invention are peptides capable of raising a specific T-cell response as measured by an ELISPOT assay, wherein more than 50 peptide specific spots per 10⁶ cells, more preferably per 10⁵, even more preferably per 10⁴ cells are measured.

Most preferred peptides according to the present invention are peptides that are capable of eliciting a cellular immune response in an individual suffering from a clinical condition characterized by the expression of mutant CALR, in particular exon 9 mutant CALR, or by the expression of mutant JAK2, in particular mutant JAK2V617F, the clinical condition preferably being a proliferative condition such as a myeloproliferative condition, for example essential thrombocythaemia, primary myelofibrosis, polycythaemia vera, or acute or chronic myeloid leukemia, preferably a malignant myeloproliferative disorder such as acute or chronic myeloid leukemia.

As described above, the HLA system represents the human major histocompatibility (MHC) system. Generally, MHC systems control a range of characteristics: transplantation antigens, thymus dependent immune responses, certain complement factors and predisposition for certain diseases. More specifically, the MHC codes for three different types of molecules, i.e. Class I, II and III molecules, which determine the more general characteristics of the MHC. Of these molecules, the Class I molecules are so-called HLA-A, HLA-B and HLA-C molecules that are presented on the surface of most nucleated cells and thrombocytes.

The peptides of the present invention are characterized by their ability to bind to (being restricted by) a particular MHC Class I HLA molecule. Thus, in one embodiment the peptide is one which is restricted by a MHC Class I HLA-A molecule including HLA-A1, HLA-A2, HLA-A3, HLA-A9, HLA-A10, HLA-A11, HLA-Aw19, HLA-A23(9), HLA-A24(9), HLA-A25(10), HLA-A26(10), HLA-A28, HLA-A29(w19), HLA-A30(w19), HLA-A31(w19), HLA-A32(w19), HLA-Aw33(w19), HLA-Aw34(10), HLA-Aw36, HLA-Aw43, HLA-Aw66(10), HLA-Aw68(28), HLA-A69(28). More simple designations are also used throughout the literature, where only the primary numeric designation is used, e.g. HLA-A19 or HLA-A24 instead of HLA-Aw19 and HLA-A24(49), respectively. In specific embodiments, the peptide of the invention is restricted a MHC Class I HLA species selected from the group consisting of HLA-A1, HLA-A2, HLA-A3, HLA-A11 and HLA-A24. In specific embodiment, the peptide of the invention is restricted a MHC Class I HLA species HLA-A2 or HLA-A3.

In further useful embodiments, the peptide of the invention is a peptide, which is restricted by a MHC Class I HLA-B molecule including any of the following: HLA-B5, HLA-B7, HLA-B8, HLA-B12, HLA-B13, HLA-B14, HLA-B15, HLA-B16, HLA-B17, HLA-B18, HLA-B21, HLA-Bw22, HLA-B27, HLA-B35, HLA-B37, HLA-B38, HLA-B39, HLA-B40, HLA-Bw41, HLA-Bw42, HLA-B44, HLA-B45, HLA-Bw46 and HLA-Bw47. In specific embodiments of the invention, the MHC Class I HLA-B species to which the peptide of the invention is capable of binding is selected from HLA-B7, HLA-B35, HLA-B44, HLA-B8, HLA-B15, HLA-B27 and HLA-B51.

In further useful embodiments, the peptide of the invention is a peptide, which is restricted by a MHC Class I HLA-C molecule including but not limited to any of the following: HLA-Cw1, HLA-Cw2, HLA-Cw3, HLA-Cw4, HLA-Cw5, HLA-Cw6, HLA-Cw7 and HLA-Cw1.

In further useful embodiments, the peptide of the invention is a peptide, which is restricted by a MHC Class II HLA molecule including but not limited to any of the following: HLA-DPA-1, HLA-DPB-1, HLA-DQA1, HLA-DQB1, HLA-DRA, HLA-DRB and all alleles in these groups and HLA-DM, HLA-DO.

The selection of peptides potentially having the ability to bind to a particular HLA molecule can be made by the alignment of known sequences that bind to a given particular HLA molecule to thereby reveal the predominance of a few related amino acids at particular positions in the peptides. Such predominant amino acid residues are also referred to herein as “anchor residues” or “anchor residue motifs”. By following such a relatively simple procedure based on known sequence data that can be found in accessible databases, peptides can be derived from exon 9 CALR or JAK2V6417, which are likely to bind to a specific HLA molecule. Representative examples of such analyses for a range of HLA molecules are given in the below table:

TABLE 2

| | | | | | | | C- |
|---|---|---|---|---|---|---|---|
| HLA allele | Position 1 | Position 2 | Position 3 | Position 5 | Position 6 | Position 7 | terminal |
| HLA-A1 | | T, S | D, E | | | L | Y |
| HLA-A2 | | L, M | | | V | | L, V |
| HLA-A3 | | L, V, M | F, Y | | | | K, Y, F |
| HLA-A11 | | V, I, F, Y | M, L, F, Y, I | | | | K, R |
| HLA-A23 | | I, Y | | | | | W, I |
| HLA-A24 | | Y | | I, V | F | | I, L, F |
| HLA-A25 | | M, A, T | I | | | | W |
| HLA-A26 | E, D | V, T, I, L, F | | | I, L, V | | Y, F |
| HLA-A28 | E, D | V, A, L | | | | | A, R |
| HLA-A29 | | E | | | | | Y, L |
| HLA-A30 | | Y, L, F, V | | | | | Y |
| HLA-A31 | | | L, M, F, Y | | | | R |
| HLA-A32 | | I, L | | | | | W |
| HLA-A33 | | Y, I, L, V | | | | | R |
| HLA-A34 | | V, L | | | | | R |
| HLA-A66 | E, D | T, V | | | | | R, K |
| HLA-A68 | E, D | T, V | | | | | R, K |
| HLA-A69 | | V, T, A | | | | | V, L |
| HLA-A74 | | T | | | | | V, L |
| HLA-B5 | | A, P | F, Y | | | | I, L |
| HLA-B7 | * | P | | | | | L, F |
| HLA-B8 | | | K | K, R | | | L |
| HLA-B14 | | R, K | | | | | L, V |
| HLA-B15 | | Q, L, K, P, | | | | | F, Y, W |
| (B62) | | H, V, I, M, | | | | | |
| | | S, T | | | | | |
| HLA-B17 | | | | | | | L, V |
| HLA-B27 | | R | | | | | Y, K, F, L |
| HLA-B35 | | P | | | | | I, L, M, Y |
| HLA-B37 | | D, E | | | | | I, L, M |
| HLA-B38 | | H | D, E | | | | F, L |
| HLA-B39 | | R, H | | | | | L, F |
| HLA-B40 | | E | F, I, V | | | | L, V, A, W, |
| (B60, 61) | | | | | | | M, T, R |
| HLA-B42 | | L, P | | | | | Y, L |
| HLA-B44 | | E | | | | | F, Y, W |
| HLA-B46 | | M, I, L, V | | | | | Y, F |
| HLA-B48 | | Q, K | | | | | L |
| HLA-B51 | | A, P, G | | | | | F, Y, I, V |
| HLA-B52 | | Q | F, Y | | | | I, V |
| HLA-B53 | | P | | | | | W, F, L |
| HLA-B54 | | P | | | | | |
| HLA-B55 | | P | | | | | A, V |
| HLA-B56 | | P | | | | | A, V |
| HLA-B57 | | A, T, S | | | | | F, W, Y |
| HLA-B58 | | A, T, S | | | | | F, W, Y |
| HLA-B67 | | P | | | | | L |
| HLA-B73 | | R | | | | | P |
| HLA-Cw1 | | A, L | | | | | L |
| HLA-Cw2 | | A, L | | | | | F, Y |
| HLA-Cw3 | | A, L | | | | | L, M |
| HLA-Cw4 | | Y, P, F | | | | | L, M, F, Y |
| HLA-Cw6 | | | | | | | L, I, V, Y |
| HLA-Cw6 | | Y | | | | | L, Y, F |
| HLA-Cw8 | | Y | | | | | L, I, |
| HLA- | | A, L | | | | | L, V |
| Cw16 | | | | | | | |

* In one embodiment there is no specific anchor residue for this position, however in a preferred embodiment the anchor residue is R or A.

Thus, as an example, nonapeptides potentially having the ability to bind to HLA-A3 would have one of the following sequences: Xaa-L-Y-Xaa-Xaa-Xaa-Xaa-Xaa-K, Xaa-L-Y-Xaa-Xaa-Xaa-Xaa-Xaa-Y; Xaa-L-Y-Xaa-Xaa-Xaa-Xaa-Xaa-F or Xaa-V-Y-Xaa-Xaa-Xaa-Xaa-Xaa-K (Xaa indicating any amino acid residue). In a similar manner, sequences potentially having the ability to bind to any other HLA molecule can be designed. It will be appreciated that the person of ordinary skill in the art will be able to identify further “anchor residue motifs” for a given HLA molecule.

The peptide of the invention may have a sequence which is a native sequence of the exon 9 mutant CALR or JAK2V617F from which it is derived, wherein the exon 9 mutant CALR or JAK2V617F may be any exon 9 mutant CALR or JAK2V617F described herein. However, peptides having a higher affinity to any given HLA molecule may be derived from such a native sequence by modifying the sequence by substituting, deleting or adding at least one amino acid residue, e.g. on the basis of the procedure described above, whereby anchor residue motifs in respect of the given HLA molecule are identified.

Thus, in useful embodiments, the polypeptides of the invention include peptides, the sequences of which comprise, for each of the specific HLA alleles listed in table 2, any of the amino acid residues as indicated in the table.

Thus, the peptides of the invention may be any of the above-mentioned peptides comprising consecutive sequences from exon 9 mutant CALR, wherein in the range of 1 to 10, preferably in the range of 1 to 5, more preferably in the range of 1 to 3, even more preferably in the range of 1 to 2, yet more preferably 1 amino acid has been exchanged for another amino acid, preferably in a manner so that the peptide comprises one or more, preferably all anchor residues of a given HLA-A specific peptide as indicated in the table above. The peptides of the invention may also be any of the above-mentioned peptides comprising consecutive sequences from JAK2V617F, wherein in the range of 1 to 10, preferably in the range of 1 to 5, more preferably in the range of 1 to 3, even more preferably in the range of 1 to 2, yet more preferably 1 amino acid has been exchanged for another amino acid, preferably in a manner so that the peptide comprises one or more, preferably all anchor residues of a given HLA-A specific peptide as indicated in the table above.

Examples of preferable HLA species, to which preferred peptides of the present invention are restricted include: a MHC Class I HLA species selected from the group consisting of HLA-A1, HLA-A2, HLA-A3, HLA-A11 and HLA-A24, more preferably the peptide is restricted by HLA-A3 or HLA-A2. Alternatively a preferred HLA species includes MHC Class I HLA-B species selected from the group consisting of HLA-B7, HLA-B35, HLA-B44, HLA-B8, HLA-B15, HLA-B27 and HLA-B51.

An approach to identifying polypeptides of the invention includes the following steps: selecting a particular HLA molecule, e.g. one occurring at a high rate in a given population, carrying out an alignment analysis as described above to identify “anchor residue motifs” in the exon 9 mutant CALR protein or in the JAK2V617F protein, isolating or constructing peptides of a suitable size that comprise one or more of the identified anchor residues and testing the resulting peptides for the capability of the peptides to elicit INF-γ-producing cells in a PBL population of a cancer patient at a frequency of at least 1 per 10⁴ PBLs as determined by an ELISPOT assay as described in Example 1. For example, the capability of the peptides to elicit INF-γ-producing cells in a PBMC population of a cancer patient has frequency of at least 1 per 10⁴ PBMCs.

In one aspect of the present invention, CALR-derived peptides, in particular exon 9 mutant CALR-derived peptides, and JAK2-derived peptides, in particular JAK2V617F-derived peptides, longer than 8 to 10 amino acid residues are provided. Polypeptides longer than 8 to 10 amino acids are processed by the proteasome to a shorter length for binding to HLA molecules. Thus, when administering a polypeptide longer than 8 to 10 amino acid residues long, the “long” polypeptide/protein/protein fragment/variant of exon 9 mutant CALR or JAK2V617F may be processed in vivo into a series of smaller peptides in the cytosol by the proteasome. An advantage of using a longer polypeptide that may be processed by the proteasome into a variety of different shorter peptides is that more HLA classes may be targeted with one peptide than one 8 to 10 amino acid peptide that is restricted to a particular HLA class.
a) Surprisingly, some of the peptides of the present invention bind to MHC molecules with an affinity sufficiently high to render substitutions unnecessary and are ready for use as antigens as they are presented here. Preferably, the vaccine composition of the present invention comprises one or more of the following: exon 9 mutant CALR protein (SEQ ID NO: 10), JAK2V617F mutant protein (SEQ ID NO: 6), polypeptide fragments thereof (SEQ ID NO: 16, SEQ ID NO: 2, SEQ ID NO: 3, SEQ ID NO: 7, SEQ ID NO: 15, SEQ ID NO: 1, SEQ ID NO: 17), likewise variants, functional homologues of full length and partial length exon 9 mutant CALR or JAK2V617F, contiguous peptides of exon 9 mutant CALR or JAK2V617F and functional homologues of these. More preferably, the vaccine composition comprises any of the sequences listed in Table 1. Very preferably, the vaccine, composition comprises the peptides SEQ ID NO: 16 (CALR₃₇₈₋₄₁₁), SEQ ID NO: 2 (CALR₃₆₇₋₃₉₆), SEQ ID NO: 3 (CALR₃₈₃₋₄₁₁, 1, SEQ ID NO: 14 (CALR₃₇₈₋₄₁₁), SEQ ID NO: 15 (CALR₃₇₃₋₃₉₃), SEQ ID NO: 1 (CALR₃₇₆₋₄₁₁), SEQ ID NO: 17 (CALR₃₆₇₋₄₁₁) or SEQ ID NO: 7 (JAK2₆₁₀₋₆₁₈).

A significant feature of the peptide of the invention is its capability to recognize or elicit INF-γ-producing responder T cells, i.e. cytotoxic T cells (CTLs) that specifically recognize the particular peptide in a PBL population, on an APC or tumor/neoplastic cells of an individual suffering from a cancer and/or an infection (target cells). This activity is readily determined by subjecting PBLs, PBMCs, APCs or tumor cells from an individual to an ELISPOT assay. Prior to the assay, it may be advantageous to stimulate the cells to be assayed by contacting the cells with the peptide to be tested. Preferably, the peptide is capable of eliciting or recognizing INF-γ-producing T cells at a frequency of at least 1 per 10⁴ PBLs such as at a frequency of at least 1 per 10⁴ PBMCs as determined by an ELISPOT assay as used herein. More preferably the frequency is at least 5 per 10⁴ PBLs, most preferably at least 10 per 10⁴ PBLs, such as at least 50 or 100 per 10⁴ PBLs. For example, the frequency is at least 5 per 10⁴ PBMCs, most preferably at least 10 per 10⁴ PBMCs, such as at least 50 or 100 per 10⁴ PBMCs.

The ELISPOT assay represents a strong tool to monitor T-cell responses specific against exon 9 mutant CALR peptide or JAK2V617F peptide. A major implication of the findings herein is that the peptides of the invention are expressed and complexed with HLA molecules on cancer cells and/or on APCs expressing exon 9 mutant CALR or JAK2V617F. This renders these cancer cells susceptible to destruction by CTLs and emphasizes the usefulness of CALR immunization to fight cancer and myeloproliferative disorders. The presence of spontaneous CTL-responses in PBLs from melanoma patients to HLA-restricted CALR derived peptide epitopes shows the immunotherapeutic potential of CALR immunogenically active peptides.

In an embodiment of the present invention the peptide of the invention is capable of eliciting INF-γ-producing cells in a PBL population of an individual suffering from a clinical condition where a mutant CALR is expressed, such as an exon 9 mutant CALR of SEQ ID NO: 10 or a functional homologue thereof having at least 70% identity to SEQ ID NO: 10, or from a clinical condition where a mutant JAK2V617F is expressed, such as JAK2V617F of SEQ ID NO: 6. The clinical condition is preferably a proliferative disorder, such as a myeloproliferative disorder, preferably a malignant myeloproliferative disorder.

Individual

The individual to be treated with the vaccine composition of the present invention is an individual suffering from a clinical condition. The individual is preferably of a mammalian species and most preferably a human being. The individual may be of any age, young or old, and may be either male or female. The clinical condition from which the individual suffers may be a neoplastic disease such as a myeloproliferative disease or a cancer.

An embodiment of the present invention provides a vaccine for the treatment, reduction of risk of, stabilization of or prevention of a cancer. In another embodiment the present invention provides a vaccine for the treatment, reduction of risk of, stabilization of or prevention of a disease stemming from an infection, such as a microbial or viral infection.

Myeloproliferative Disorders

The vaccine composition of the present invention may be used to prevent, reduce the risk of or treat a clinical condition. Preferably, the clinical condition is associated with or characterized by the expression of an exon 9 mutant CALR and/or mutant JAK2V617F. The exon 9 mutant CALR may be the exon 9 mutant CALR as identified in SEQ ID NO: 10 or may be a homolog sharing at least 70% identity therewith in their wild type forms, but need not be functional. The exon 9 mutant may be any of the mutants listed in FIG. 3, panel A, p. 2400 of Nangalia et al. For example, the exon 9 CALR mutant may be L367fs*46 (full length set forth in SEQ ID NO: 10), E370fs*43, E370fs*48, L367fs*48, L367fs*44, K368fs*51, L367fs*52, R366fs*53, E371fs*49, K368fs*43, E370fs*37, D373fs*47, K374fs*53, E371fs*49, K385fs*47, K385fs*47, R376fs*55, K385fs*47 or E381fs*48. JAK2V617F may be the JAK2V617F mutant as identified in SEQ ID NO: 6, or may be a homolog sharing at least 70% identity therewith in their wild type forms, but need not be functional. It is understood hereby that the expression level of exon 9 mutant CALR or mutant JAK2V617F (the expression being expression of e.g. hnRNA, mRNA, precursor protein, fully processed protein) in an individual suffering from a clinical condition is the same or higher than in an individual not suffering from a clinical condition.

In one embodiment of the invention the clinical condition is a proliferative disorder, such as a myeloproliferative disorder, such as a preneoplastic or neoplastic disorder. In a preferred embodiment of the invention, the clinical condition is cancer. Cancer (malignant neoplasm) is a class of diseases in which a group of cells display the traits of uncontrolled growth (growth and division beyond the normal limits), invasion (intrusion on and destruction of adjacent tissues), and sometimes metastasis (spread to other locations in the body via lymph or blood). These three malignant properties of cancers differentiate them from benign tumors, which are self-limited, do not invade or metastasize. Most cancers form a tumor but some, like leukemia, do not.

A non-limiting group of myeloproliferative neoplasms that may be treated or prevented include essential thrombocythaemia, polycythaemia vera, primary myelofibrosis, and acute or chronic myeloid leukemia.

In a preferred embodiment the vaccine composition according to the invention is capable of eliciting a clinical response in subject, wherein the clinical response may be characterized by a stable disease, in a preferred embodiment the clinical response may be characterized by a partial response or preferably the clinical response may be characterized by complete remission of a disorder such as a cancer.

In one aspect of the invention the vaccine composition is capable of eliciting a clinical response in an individual. In one embodiment the clinical response may be characterized by a stable disease (no further worsening or progression), in a preferred embodiment the clinical response may be characterized by a partial response or preferably the clinical response may be characterized by complete remission of a disorder such as a cancer. The clinical response may be determined as described herein below.

In another aspect of the invention the vaccine composition is capable of eliciting a clinical response in subject, wherein the clinical response is characterized by a decrease in the sum of the longest diameter of the largest target lesion. The decrease may be determined as described herein below.

European Leukemia Net MPN Essential Thrombocytemia Response Criteria: Clinico-Hematological Response:
Complete Response:
   1. Thrombocyte concentration in peripheral blood <400×10⁹/L
   2. No disease related symptoms
   3. Normal size of liver and spleen as assessed by computer tomography or sonography.
   4. Leucocyte concentration in peripheral blood <10×10⁹/L
Partial response: Criteria for complete response not met, but thrombocyte concentration in peripheral blood <600×10⁹/L or reduction of thrombocyte counts by >50% of the baseline value.
No response: Criteria for complete or partial response not met.

Molecular Response:
Complete response: Specific molecular abnormity unmeasurable.
Partial response: (For patients with mutant allelic burden >10%). A reduction of >50% in mutant allelic burden for patients with a mutant allelic burden of <50% at baseline. A reduction of >25% in mutant allelic burden for patients with a mutant allelic burden of >50% at baseline.
No response: Criteria for complete or partial response not met.

Histological Response:

Remission in bone marrow defined as no megakaryocyte hyperplasia.

European Leukemia Net MPNpolycythemia Vera Response Criteria:

Clinico-Hematological Response:
Complete response:
   1. Hematocrit <45% without phlebotomy
   2. Thrombocyte concentration in peripheral blood <400×10⁹/L
   3. No disease related symptoms
   4. Normal size of liver and spleen as assessed by computer tomography or sonography.
   5. Leucocyte concentration in peripheral blood <10×10⁹/L
Partial response: Patients not fulfilling to criteria for complete response:
   1. Hematocrit <45% without phlebotomy OR
   2. Response in three or more criteria from above.
No response: Not meeting response for neither complete nor partial response.

Molecular Response:
Complete response: Unmeasurable molecular abormity (eg. JAK2V617F mutant allelic burden)
Partial response: (For patients with mutant allelic burden >10%). A reduction of >50% in mutant allelic burden for patients with a mutant allelic burden of <50% at baseline. A reduction of >25% in mutant allelic burden for patients with a mutant allelic burden of >50% at baseline.
No response: Criteria for complete or partial response not met.

Response to myelofibrosis is assessed following the criteria as stated by Tefferi et al. in Blood, 2013. (Tefferi et al. Revised response criteria for myelofibrosis: International Working Group-Myeloproliferative Neoplasms Research and Treatment (IWG-MRT) and European LeukemiaNet (ELN) consensus report. Blood. 2013, 122; (8): 1395-1398)

It is contemplated that the vaccine composition of the invention is capable of eliciting an immune response against a disorder associated with expression of an exon 9 mutant CALR, for example the mutant CALR of SEQ ID NO: 10 or a functional homologue thereof having at least 70% identity to SEQ ID NO: 10, or associated with expression of mutant JAK2V617F of SEQ ID NO: 6 or a functional homologue thereof having at least 70% identity to SEQ ID NO: 6, when administered to an individual suffering from a disorder associated with expression of an exon 9 mutant CALR and/or of JAK2V617F. The vaccine composition of the invention is capable of eliciting the production in a vaccinated individual of effector T-cells having a cytotoxic effect against the neoplastic cells, APCs expressing an exon 9 mutant CALR and/or mutant JAK2V617F and/or inducing infiltration of antigen specific T-cells in tumor stroma in a subject.

In addition to their capacity to elicit immune responses in PBL populations it is also contemplated that the peptides of the invention are capable of eliciting cytolytic immune responses in situ, i.e. in solid tumor tissues. This may for example be demonstrated by providing HLA-peptide complexes, e.g. being multimerized and being provided with a detectable label, and using such complexes for immunohistochemistry stainings to detect in a tumor tissue CTLs that are reactive with the epitope peptide of the invention. Accordingly, a further significant feature of the peptide of the invention is that it is capable of in situ detection in a tumor tissue of CTLs that are reactive with the epitope peptide.

It is also contemplated that the peptides of the invention, in addition to their capacity to bind to HLA molecules resulting in the presentation of complexes of HLA and peptides on cell surfaces, which complexes in turn act as epitopes or targets for cytolytic T cells, may elicit other types of immune responses, such as B-cell responses resulting in the production of antibodies against the complexes and/or a Delayed Type Hypersensitivity (DTH) reaction. The latter type of immune response is defined as a redness and palpable induration at the site of injection of the peptide of the invention.

It is an object of the presenting invention to provide a vaccine composition comprising an exon 9 mutant of CALR of SEQ ID NO: 10 or a mutant JAK2V617F of SEQ ID NO: 6, or a functional homologue of an exon 9 mutant CALR having at least 70% identity to SEQ ID NO: 10, or a functional homologue of JAK2V617F having at least 70% identity to SEQ ID NO: 6, or an immunogenically active peptide fragment comprising a consecutive sequence of said exon 9 mutant CALR or JAK2V617F or said functional homologue thereof or a nucleic acid encoding said exon 9 mutant CALR or JAK2V617F or said peptide fragment; and an adjuvant, for the prevention of, reduction of risk from or treatment of a myeloproliferative disorder, in particular a malignant myeloproliferative disorder.

Cancer Combination Treatment

In some cases it will be appropriate to combine the treatment method of the invention with a further cancer treatment such as chemotherapy, radiotherapy, treatment with immunostimulating substances, gene therapy, treatment with antibodies and treatment using dendritic cells.

The combination of a CALR- or JAK2-based immunotherapy as disclosed by the present invention with cytotoxic chemotherapy and or another anti-cancer immunotherapeutic treatment is an effective approach to treat cancer. These remedies are also referred to herein as “second active ingredients”.

Examples of anti-neoplastic or chemotherapeutic agents that are of relevance in regards to co-administration (sequentially or simultaneously) with the vaccine composition of the present invention include, but are not limited to: all-trans retinoic acid, Actimide, Anagrelide, Azacitidine, Busulphan, Azathioprine, Bleomycin, Carboplatin, Capecitabine, Cisplatin, Chlorambucil, Cyclophosphamide, Cytarabine, Daunorubicin, Docetaxel, Doxifluridine, Doxorubicin, Epirubicin, Etoposide, Fludarabine, Fluorouracil, Gemcitabine, Hydroxyurea, Idarubicin, Irinotecan, Lenalidomide, Leucovorin, Mechlorethamine, Melphalan, Mercaptopurine, Methotrexate, Mitoxantrone, Oxaliplatin, Paclitaxel, Pegylated Interferon-alpha, Pemetrexed, Revlimid, Ruxolitinib, Temozolomide, Teniposide, Thioguanine, Valrubicin, Vinblastine, Vincristine, Vindesine and Vorinostat and Vinorelbine. In one embodiment, a chemotherapeutic agent for use in the combination of the present agent may, itself, be a combination of different chemotherapeutic agents. Suitable combinations include FOLFOX and IFL. FOLFOX is a combination which includes 5-fluorouracil (5-FU), leucovorin, and oxaliplatin. IFL treatment includes irinotecan, 5-FU, and leucovorin.

Another second active ingredient may be a kinase inhibitor, for separate, simultaneous or combined use in the treatment of tumors. In this regard the tyrosine kinase inhibitor—ruxolitinib—may be an option. Suitable kinase inhibitors include those which have been shown to possess anti-tumor activity (such as gefitinib (Iressa) and erlotinib (Tarceva) and these could be used in combination with the peptides. The receptor tyrosine kinase inhibitors, such as Sunitinib malate and Sorafenib which have been shown to be effective in the treatment of renal cell carcinoma are also suitable to be used as second active ingredients.

Further examples of second active ingredients are immunostimulating substances e.g. cytokines and antibodies. Such as cytokines may be selected from the group consisting of, but not limited to: GM-CSF, type I IFN, interleukin 21, interleukin 2, interleukin 12 and interleukin 15. The antibody is preferably an immunostimulating antibody such as anti-CD40, anti-PD1 antibodies or anti-CTLA-4 antibodies. The immunostimulatory substance may also be a substance capable of depletion of immune inhibitory cells (e.g. regulatory T-cells) or factors, said substance may for example be E3 ubiquitin ligases. E3 ubiquitin ligases (the HECT, RING and U-box proteins) have emerged as key molecular regulators of immune cell function, and each may be involved in the regulation of immune responses during infection by targeting specific inhibitory molecules for proteolytic destruction. Several HECT and RING E3 proteins have now also been linked to the induction and maintenance of immune self-tolerance: c-Cbl, Cbl-b, GRAIL, Itch and Nedd4 each negatively regulate T cell growth factor production and proliferation.

In an embodiment, the vaccine composition of the present invention, comprising a CALR-derived polypeptide or a JAK2-derived polypeptide, wherein the polypeptide is derived from an exon 9 mutant of CALR or from JAK2V617F, is administered in combination with a second active ingredient, such as an immunostimulatory substance. The immunostimulatory substance is preferably an interleukin such as pegylated interferon-alpha, IL-21 or IL-2 or a chemotherapeutic agent. In some embodiments, the target cells are treated with IFN-γ prior to administering the vaccine composition. In some embodiments, IFN-γ is administered to the subject to be treated prior to administering the vaccine composition.

The vaccine compositions of the invention may also comprise one or more additional antigens in addition to polypeptides derived from exon 9 mutant CALR or JAK2V617F. Said antigens, may for example be immunogenically active peptides derived from cancer associated proteins.

Thus, the vaccine compositions of the invention may in addition to polypeptides derived from exon 9 mutant CALR or JAK2V617F and/or immunogenically active peptide fragments thereof also comprise one or more of the following:
1) Indoleamine-2,3-dioxygenase (IDO)
2) An immunogenically active peptide fragment of IDO
3) A functional homologue of 1) or 2)
4) A polypeptide comprising 1), 2) or 3)
5) A nucleic acid encoding any of 1), 2), 3) or 4).

Said IDO may in particular be IDO of SEQ ID NO: 1 of WO 2009/143843, IDO of SEQ ID NO: 13 of WO 2009/143843, IDO of SEQ ID NO: 14 of WO 2009/143843, IDO of SEQ ID NO: 15 of WO 2009/143843 or IDO of SEQ ID NO: 1 of WO 2009/143843. Useful immunogenically active peptide fragments of IDO, which can be contained in the vaccine compositions of the present invention are described in WO 2009/143843.

The vaccine compositions of the invention may in addition to polypeptides derived from exon 9 mutant CALR or JAK2V617F and/or immunogenically active peptide fragments thereof also comprise one or more of the following:
1) PD-L1
2) An immunogenically active peptide fragment of PD-L1
3) A functional homologue of 1) or 2)
4) A polypeptide comprising 1), 2) or 3)
5) A nucleic acid encoding any of 1), 2), 3) or 4).

Said PD-L1 may in particular be PD-L1 of SEQ ID NO:1 of WO2013/056716. Useful immunogenically active peptide fragments of PD-L1, which can be contained in the vaccine compositions of the present invention, are described in WO2013/056716.

The vaccine compositions of the invention may in addition to polypeptides derived from exon 9 mutant CALR or JAK2V617F and/or immunogenically active peptide fragments thereof also comprise one or more of the following:
1) tryptophan 2,3-dioxygenase (TDO)
2) An immunogenically active peptide fragment of TDO
3) A functional homologue of 1) or 2)
4) A polypeptide comprising 1), 2) or 3)
5) A nucleic acid encoding any of 1), 2), 3) or 4).

Said TDO may in particular be TDO of SEQ ID NO: 1 of pending application “Vaccine compositions comprising Tryptophan 2,3-dioxygenase or fragments thereof” filed by the present inventors. Useful immunogenically active peptide fragments of TDO, which can be contained in the vaccine compositions of the present invention, are described in said pending application.

Pharmaceutical Compositions

The present invention regards pharmaceutical compositions capable of treating, reducing the risk of and/or preventing a clinical disorder associated with expression of mutant CALR, in particular an exon 9 mutant CALR, or mutant JAK2, in particular JAK2V617F, in an individual. Said pharmaceutical composition may in particular be a vaccine composition. The vaccine compositions of the present invention may be “traditional” vaccine compositions comprising antigens such as proteins polypeptides and/or nucleic acid molecules. They may also be in the form of compositions comprising cells, such as modified cells originating from the individual and later processed, or to compositions comprising complex molecules such as antibodies or TCRs. In specific embodiments, the present vaccines are in the form of compositions comprising peptides as disclosed herein or cells.

Generally, a vaccine is a substance or composition capable of inducing an immune response in an individual. The composition may comprise one or more of the following: an “active component” such as an antigen(s) (e.g. protein, polypeptides, peptides, nucleic acids and the like), nucleic acid constructs comprising one or more antigens amongst other elements, cells, (e.g. loaded APC, T cells for adoptive transder aso.), complex molecules (Antibodies, TCRs and MHC complexes and more), carriers, adjuvants and pharmaceutical carriers. In the following, the various components of a vaccine composition according to the present invention are disclosed in more detail.

The vaccine composition of the invention is capable of eliciting an immune response against a neoplastic or a cancer cell, a DC or an APC expressing exon 9 mutant CALR of SEQ ID NO: 10 or a functional homologue thereof having at least 70% identity to SEQ ID NO: 10, or expressing JAK2V617F of SEQ ID NO: 6 or a functional homologue thereof having at least 70% identity to SEQ ID NO: 6, when administered to an individual suffering from a myeloproliferative disorder or an MPN (leading to the expression of exon 9 mutant CALR and/or mutant JAK2V617F). In a preferred embodiment the clinical condition is acute or chronic myeloid leukemia. The vaccine composition of the invention is capable of eliciting the production in a vaccinated individual of effector T-cells having a cytotoxic effect against neoplastic or cancer cells, APCs and DCs expressing exon 9 mutant CALR and/or mutant JAK2V617F. In some embodiments, the vaccine elicits production of effector T-cells specifically recognizing any of the peptides described herein. The vaccine composition may also be capable of eliciting the production in a vaccinated individual of tumour infiltrating lymphocytes specific for the peptides of the present disclosure.

Antigens and Other Active Components

Protein/Polypeptide Based Vaccine Compositions

The peptides of the present invention are ready for use as antigens as they are presented here. Preferably, the vaccine composition of the present invention comprises one or more of the following:
1) An exon 9 mutant Calreticulin CALR, which may be any of the CALRs described herein in the section “Calreticulin (CALR)”, or a JAK2 mutant, in particular JAK2V617F, as detailed in the section “Janus kinase 2 (JAK2)”;
2) An immunogenically active peptide fragment of exon 9 mutant CALR or JAK2V617F comprising a consecutive sequence of amino acids of an exon 9 mutant CALR or JAK2V617F, which may be any of the peptides described herein below in the sections “Immunogenically active peptide fragments of exon 9 mutant CALR and JAK2V617F”;
3) An immunogenically active peptide fragment of an exon 9 mutant CALR or JAK2V617F, which is an MHC Class I-restricted peptide fragment or MHC Class II-restricted peptide fragment, such as any of the an MHC Class I-restricted peptide fragments or MHC Class II-restricted peptide fragments described in the section “MHC”;
4) A functional homologue of the polypeptides under 1), 2) and 3);
5) A polypeptide comprising any of polypeptides under 1), 2), 3) and 4), which may be any of the polypeptides described herein above in the section “Polypeptides comprising exon 9 mutant CALR or JAK2V617F or a fragment thereof;
6) A nucleic acid encoding any of the polypeptides under 1), 2), 3) and 4).

The choice of antigen in the vaccine composition of the invention will depend on parameters determinable by the person of skill in the art. As it has been mentioned, each of the different peptides of the invention is presented on the cell surfaces by a particular HLA molecule. As such, if a subject to be treated is typed with respect to HLA phenotype, a peptide/peptides are selected that is/are known to bind to that particular HLA molecule. Alternatively, the antigen of interest is selected based on the prevalence of the various HLA phenotypes in a given population. As an example, HLA-A2 is the most prevalent phenotype in the Caucasian population, and therefore, a composition containing a peptide binding to HLA-A2 will be active in a large proportion of that population. Furthermore, the antigens/peptides of the present invention may be modified according to the anchor residue motifs presented in Table 2, to enhance binding to particular HLA molecules.

The composition of the invention may also contain a combination of two or more immunogenically active peptide fragments of exon 9 mutant CALR or JAK2V617F e.g. any of the peptides described in the sections “Immunogenically active peptide fragments of exon 9 mutant CALR or JAK2V617F”, “Polypeptides comprising exon 9 mutant CALR or JAK2V617F or a fragment thereof” and “MHC”. Said immunogenically active peptide fragments of CALR or JAK2 may each interact specifically with a different HLA molecule so as to cover a larger proportion of the target population. Thus, as examples, the pharmaceutical composition may contain a combination of a peptide restricted by a HLA-A molecule and a peptide restricted by a HLA-B molecule, e.g. including those HLA-A and HLA-B molecules that correspond to the prevalence of HLA phenotypes in the target population, such as e.g. HLA-A2 and HLA-B35. Additionally, the composition may comprise a peptide restricted by an HLA-C molecule.

In the case of peptide-based vaccines, epitopes can be administered in an ‘MHC-ready’ form, which enables presentation through exogenous loading independently of antigen uptake and processing by host antigen-presenting cells. The peptides of the present invention comprise both peptides in a short ‘MHC-ready’ form and in a longer form requiring processing by the proteasome thus providing a more complex vaccine composition that can target multiple tumor antigens. The more different HLA groups are targeted by a vaccine, the higher likelihood of the vaccine functioning in diverse populations.

Multi Epitope Vaccine Composition

The invention also relates to highly immunogenic multi-epitope vaccines. Preferably, such vaccines should be designed so as to facilitate a simultaneous delivery of the best-suited immunogenically active peptide fragments of exon 9 mutant CALR or JAK2V617F optionally in combination with other suitable peptides, or with each other, and/or adjuvants as described hereinafter. The present invention encompasses such multi-epitope vaccines comprising immunogenically active peptide fragments of exon 9 mutant CALR or JAK2V617F optionally in combination with further proteins or peptides fragments not belonging to or derived from exon 9 mutant CALR or JAK2V617F and/or adjuvants as described hereinafter. An important factor driving the development of vaccines having a more complex composition is the desire to target multiple tumor antigens e.g. by designing vaccines comprising or encoding a collection of carefully selected CTL and Tₕ cell epitopes. The invention thus in one aspect relates to vaccine compositions comprising both Class I and Class II-restricted exon 9 mutant CALR or JAK2V617F epitopes.

The peptides of the present invention thus comprise both peptides in a short ‘MHC-ready’ form (class I restricted), and in a longer form requiring processing by the proteasome (class II restricted). Thus, the composition according to the present invention may be provided as a multi-epitope vaccine comprising class I restricted epitope and/or class II restricted epitopes as defined hereinbefore.

Nucleic Acid Based Vaccine Composition

The vaccine composition according to the present invention may comprise a nucleic acid encoding a CALR or JAK2 polypeptide or an immunologically active peptide fragment thereof. Said nucleic acid may thus encode any of the above-mentioned proteins and peptide fragments. The nucleic acid may for example be DNA, RNA, LNA, HNA, PNA, preferably the nucleic acid is DNA or RNA.

The nucleic acids of the invention may be comprised within any suitable vector, such as an expression vector. Numerous vectors are available and the skilled person will be able to select a useful vector for the specific purpose. The vector may, for example, be in the form of a plasmid, cosmid, viral particle or artificial chromosome. The appropriate nucleic acid sequence may be inserted into the vector by a variety of procedures, for example, DNA may be inserted into an appropriate restriction endonuclease site(s) using techniques well known in the art. Apart from the nucleic acid sequence according to the invention, the vector may furthermore comprise one or more of a signal sequence, an origin of replication, one or more marker genes, an enhancer element, a promoter, and a transcription termination sequence. The vector may also comprise additional sequences, such as enhancers, poly-A tails, linkers, polylinkers, operative linkers, multiple cloning sites (MCS), STOP codons, internal ribosomal entry sites (IRES) and host homologous sequences for integration or other defined elements. Methods for engineering nucleic acid constructs are well known in the art (see, e.g., Molecular Cloning: A Laboratory Manual, Sambrook et al., eds., Cold Spring Harbor Laboratory, 2nd Edition, Cold Spring Harbor, N.Y., 1989). The vector is preferably an expression vector, comprising the nucleic acid operably linked to a regulatory nucleic acid sequence directing expression thereof in a suitable cell. Within the scope of the present invention said regulatory nucleic acid sequence should in general be capable of directing expression in a mammalian cell, preferably a human cell, more preferably in an antigen presenting cell.

In one preferred embodiment the vector is a viral vector. The vector may also be a bacterial vector, such as an attenuated bacterial vector. Attenuated bacterial vectors may be used in order to induce lasting mucosal immune responses at the sites of infection and persistence. Different recombinant bacteria may be used as vectors, for example the bacterial vector may be selected from the group consisting of Salmonella, Lactococcus], and Listeria. In general, induction of immunity to the heterologous antigen HPV16 L1 or E7 could be shown, with strong CTL induction and tumor regression in mice. The vector may furthermore comprise a nucleic acid encoding a T-cell stimulatory polypeptide.

Loaded APCs

In useful embodiments an immunogenic response directed against a cancer disease is elicited by administering the peptide of the invention either by loading MHC class I or class II molecules on antigen presenting cells (APCs) from the individual, by isolating PBLs from the individual and incubating the cells with the peptide prior to injecting the cells back into the individual or by isolating precursor APCs from the individual and differentiating the cells into professional APCs using cytokines and antigen before injecting the cells back into the individual.

It is thus an aspect of the invention to provide vaccine compositions comprising antigen presenting cells comprising CALR or JAK2 or an immunologically active peptide fragment thereof or a nucleic acid encoding said protein or said immunologically active peptide fragment. The antigen presenting cell may be any cell capable of presenting an antigen to a T-cell. Preferred antigen presenting cells are dendritic cells. The dendritic cells (DC) may be prepared and used in therapeutic procedure according to any suitable protocol, for example as described herein below. It will be appreciated by the person skilled in the art that the protocol may be adopted to use with individuals with different HLA type and different diseases.

Dendritic cells (DC) may be pulsed with 50 μg/ml HLA-restricted peptide (synthesized at GMP quality) for 1 h at 37° C. peptide and 5×10⁶ cells are administered subcutaneously at day 1 and 14, subsequently every 4 weeks, additional leukapheresis after 5 vaccinations. The generation of DC for clinical use and quality control can be performed essentially as described in Nicolette et al. (2007).

Thus, in one embodiment of the present invention, a method for treating an individual suffering from a clinical condition characterized by the expression of CALR or JAK2, preferably wherein the clinical condition is myeloproliferative, such as a myeloproliferative cancer, is one wherein the peptide is administered by presenting the peptide to the individual's antigen presenting cells (APCs) ex vivo followed by injecting the thus treated APCs back into the individual. There are at least two alternative ways of performing this. One alternative is to isolate APCs from the individual and incubate (load) the MHC class I molecules with the peptide. Loading the MHC class I molecules means incubating the APCs with the peptide so that the APCs with MHC class I molecules specific for the peptide will bind the peptide and therefore be able to present it to T cells. Subsequently, the APCs are re-injected into the individual. Another alternative way relies on the recent discoveries made in the field of dendritic cell biology. In this case, monocytes (being dendritic cell precursors) are isolated from the individual and differentiated in vitro into professional APC (or dendritic cells) by use of cytokines and antigen. Subsequently, the in vitro generated DCs are pulsed with the peptide and injected into the individual.

Adoptive Immunotherapy/Adoptive Transfer

An important aspect the invention relates to cultivating T-cells specific for exon 9 mutant CALR or JAK2V617F in vitro and adoptive transfer of these to individuals. Adoptive transfer means that the physician directly transfers the actual components of the immune system that are already capable of producing a specific immune response, into an individual. As shown in the examples, a specific immune response can be raised in healthy subjects against exon 9 mutant CALR or JAK2V617F or peptide fragments thereof.

It is one objective to the present invention to provide T-cells specific for exon 9 mutant CALR or JAK2V617F, which may be useful for example for adoptive transfer. Isolated T-cells comprising T-cell receptors capable of binding specifically to exon 9 mutant CALR or JAK2V617F peptide/MHC class I or exon 9 mutant CALR or JAK2V617F peptide/MHC class II complexes can be adoptively transferred to individuals, said T-cells preferably being T-cells that have been expanded in vitro, wherein the exon 9 mutant CALR or JAK2V617F peptide may be any of the immunogenically active peptide fragments of exon 9 mutant CALR or JAK2V617F mentioned herein above. Methods of expanding T-cells in vitro are well known to the skilled person. The invention also relates to methods of treatment comprising administering T-cells comprising T-cell receptors capable of binding specifically to a MHC-restricted exon 9 mutant CALR or JAK2V617F peptide complex to an individual, such as a human being suffering from a cancer disease, wherein the peptide derived from exon 9 mutant CALR or JAK2V617F may be any of the peptides mentioned herein above. The invention furthermore relates to use of T-cells comprising T-cell receptors capable of binding specifically to any of the exon 9 mutant CALR or JAK2V617F or peptide fragments thereof as described herein for the preparation of a medicament for the treatment of a cancer or infection. Autologous T-cell transfer may be performed essentially as described in Walter et al. (1995). In some embodiments, the T-cells are cytotoxic T-cells which specifically recognize exon 9 mutant CALR or JAK2V617F.

TCR Transfer

In yet another embodiment, such T-cells could be irradiated before adoptive transfer to control proliferation in the individual. It is possible to genetically engineer the specificity of T cells by TCR gene transfer (Engels et al., 2007). This allows the transfer of T cells bearing exon 9 mutant CALR peptide specificity or JAK2V617F peptide specificity into individuals. In general, the use of T cells for adoptive immunotherapy is attractive because it allows the expansion of T cells in a tumor- or virus-free environment, and the analysis of T cell function prior to infusion. The application of TCR gene-modified T cells (such as T-cells transformed with an expression construct directing expressing of a heterologous TCR) in adoptive transfer has several advantages in comparison to the transfer of T cell lines: (i) the generation of redirected T cells is generally applicable. (ii) High-affinity or very high-affinity TCRs can be selected or created and used to engineer T cells. (iii) High-avidity T cells can be generated using codon optimized or murinized TCRs allowing better surface expression of the stabilized TCRs. Genetic engineering of T cell specificity by T cell receptor (TCR) gene transfer may be performed essentially as described in Morgan et al. (2006).

TCR Transfection

TCR with known anti-tumor reactivity can be genetically introduced into primary human T lymphocytes. Genes encoding TCR alpha and beta chains from a tumor specific CTL clone can be transfected into primary T cells and in this way reprogram T cells with specificity against the tumor antigen. TCR RNA is transfected into PBL by electroporation (Schaft et al., 2006). Alternatively, T cells can be provided with at new specificity by TCR gene transfer using retroviral vectors (Morgan et al., 2006). However, the provirus from the retroviral vector might integrate at random in the genome of the transfected cells and subsequently disturb cell growth. Electroporation of T cells with TCR-coding RNA overcome this disadvantage, since RNA is only transiently present in the transfected cells and cannot be integrated in the genome (Schaft et al., 2006). Furthermore, transfection of cells is routinely used in the laboratory.

Adjuvants and Carriers

The vaccine composition according to the invention preferably comprises an adjuvant and/or a carrier. Examples of useful adjuvants and carriers are given herein below. Thus the exon 9 mutant CALR or JAK2V617F polypeptide, the immunogenically active peptide fragments of exon 9 mutant CALR or JAK2V617F or functional homologues thereof, the polypeptides comprising same or nucleic acid encoding same may in a composition of the present invention be associated with an adjuvant and/or a carrier.

Adjuvants are any substance whose admixture into the vaccine composition increases or otherwise modifies the immune response to the exon 9 mutant CALR or JAK2V617F or to immunogenically active peptide fragments of exon 9 mutant CALR or JAK2V617F, see further in the below. Carriers are scaffold structures, for example a polypeptide or a polysaccharide, to which the exon 9 mutant CALR or JAK2V617F or peptide fragment thereof is capable of being associated and which aids in the presentation of especially the peptides of the present invention.

Many of the peptides of the invention are relatively small molecules and it may therefore be required in compositions as described herein to combine the peptides with various materials such as adjuvants and/or carriers, to produce vaccines, immunogenic compositions, etc. Adjuvants, broadly defined, are substances which promote immune responses. A general discussion of adjuvants is provided in Goding, Monoclonal Antibodies: Principles & Practice (2nd edition, 1986) at pages 61-63. Goding notes, that when the antigen of interest is of low molecular weight, or is poorly immunogenic, coupling to an immunogenic carrier is recommended. Examples of such carrier molecules include keyhole limpet haemocyanin, bovine serum albumin, ovalbumin and fowl immunoglobulin. Various saponin extracts have also been suggested to be useful as adjuvants in immunogenic compositions. It has been proposed to use granulocyte-macrophage colony stimulating factor (GM-CSF), a well known cytokine, as an adjuvant (WO 97/28816).

A carrier may be present independently of an adjuvant. The function of a carrier can for example be to increase the molecular weight of in particular peptide fragments in order to increase their activity or immunogenicity, to confer stability, to increase the biological activity, or to increase serum half-life. Furthermore, a carrier may aid in presenting the exon 9 mutant CALR or JAK2V617F protein, polypeptide, functional homologue or peptide fragments thereof to T-cells. The carrier may be any suitable carrier known to a person skilled in the art, for example a protein or an antigen presenting cell. A carrier protein could be, but is not limited to, keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier must be a physiologically acceptable carrier acceptable to humans and safe. However, tetanus toxoid and/or diptheria toxoid are suitable carriers in one embodiment of the invention. Alternatively, the carrier may be dextrans for example sepharose.

Thus it is an aspect of the present invention that the exon 9 mutant CALR or JAK2V617F protein, polypeptide fragment, variant or peptide derived here from present in the composition is associated with a carrier such as e.g. a protein of the above or an antigen-presenting cell such as e.g. a dendritic cell (DC).

Adjuvants could for example be selected from the group consisting of: AlK(SO₄)₂, AlNa(SO₄)₂, AlNH₄ (SO₄), silica, alum, Al(OH)₃, Ca₃ (PO₄)₂, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-sn-glycero-3-hydroxphosphoryloxy)-ethylamine (CGP 19835A, also referred to as MTP-PE), R1131 (MPL+TDM+CWS) in a 2% squalene/Tween-80® emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, Titermax, ISCOMS, Quil A, ALUN (see U.S. Pat. Nos. 58767 and 5,554,372), Lipid A derivatives, choleratoxin derivatives, HSP derivatives, LPS derivatives, synthetic peptide matrixes or GMDP, Interleukin 1, Interleukin 2, Montanide ISA-51 and QS-21. Preferred adjuvants to be used with the invention include oil/surfactant based adjuvants such as Montanide adjuvants (available from Seppic, Belgium), preferably Montanide ISA-51. Other preferred adjuvants are bacterial DNA based adjuvants, such as adjuvants including CpG oligonucleotide sequences. Yet other preferred adjuvants are viral dsRNA based adjuvants, such as poly I:C. Imidazochinilines are yet another example of preferred adjuvants. The most preferred adjuvants are adjuvants suitable for human use.

Montanide adjuvants (all available from Seppic, Belgium), may be selected from the group consisting of Montanide ISA-51, Montanide ISA-50, Montanide ISA-70, Montanide ISA-206, Montanide ISA-25, Montanide ISA-720, Montanide ISA-708, Montanide ISA-763A, Montanide ISA-207, Montanide ISA-264, Montanide ISA-27, Montanide ISA-35, Montanide ISA 51 F, Montanide ISA 016D and Montanide IMS, preferably from the group consisting of Montanide ISA-51, Montanide IMS and Montanide ISA-720, more preferably from the group consisting of Montanide ISA-51. Montanide ISA-51 (Seppic, Inc.) is oil/surfactant based adjuvants in which different surfactants are combined with a non-metabolizable mineral oil, a metabolizable oil, or a mixture of the two. They are prepared for use as an emulsion with an aqueous solution comprising CALR or JAK2 or a peptide fragment thereof. The surfactant is mannide oleate. QS-21 (Antigenics; Aquila Biopharmaceuticals, Framingham, Mass.) is a highly purified, water-soluble saponin that handles as an aqueous solution. QS-21 and Montanide ISA-51 adjuvants can be provided in sterile, single-use vials.

The well-known cytokine GM-CSF is another preferred adjuvant of the present invention. GM-CSF has been used as an adjuvant for a decade and may preferably be GM-CSF as described in WO 97/28816.

Desirable functionalities of adjuvants capable of being used in accordance with the present invention are listed in the below table.

TABLE 3
Modes of adjuvant action

| Action | Adjuvant type | Benefit |
|---|---|---|
| 1. Immuno- | Generally small molecules or | Upregulation of immune |
| modulation | proteins which modify the | response. Selection of Th1 or |
| | cytokine network | Th2 |
| 2. Presentation | Generally amphipathic molecules | Increased neutralizing antibody |
| | or complexes which interact with | response. Greater duration of |
| | immunogen in its native | response |
| | conformation | |
| 3. CTL | Particles which can bind or | Cytosolic processing of protein |
| induction | enclose immunogen and | yielding correct class 1 |
| | which can fuse with or disrupt | restricted peptides |
| | cell membranes | |
| | w/o emulsions for direct | Simple process if promiscuous |
| | attachment of peptide to cell | peptide(s) known |
| | surface MHC-1 | |
| 4. Targeting | Particulate adjuvants which | Efficient use of adjuvant and |
| | bind immunogen. Adjuvants | immunogen |
| | which saturate Kupffer cells | |
| | Carbohydrate adjuvants which | As above. May also determine |
| | target lectin receptors on | type of response if targeting |
| | macrophanges and DCs | selective |
| 5. Depot | w/o emulsion for short | Efficiency |
| Generation | term | Potential for singe dose |
| | Microspheres or nanospheres for | vaccine |
| | long term | |

Source: Cox, J. C., and Coulter, A. R. (1997). Vaccine 15, 248-56.

A vaccine composition according to the present invention may comprise more than one adjuvant. Furthermore, the invention encompasses a therapeutic composition further comprising any adjuvant substance and/or carrier including any of the above or combinations thereof. It is also contemplated that the CALR or JAK2 protein, variants or peptide fragments thereof, and the adjuvant can be administered separately in any appropriate sequence. Preferably, the vaccine compositions of the present invention comprise a Montanide adjuvant such as Montanide ISA 51 or Montanide ISA 720 or the GM-CSF adjuvant.

Accordingly, the invention encompasses a therapeutic composition further comprising an adjuvant substance including any of the above or combinations thereof. It is also contemplated that the antigen, i.e. the peptide of the invention and the adjuvant can be administered simultaneously or separately in any appropriate sequence.

Doses and Administration

The amount of exon 9 mutant CALR or JAK2V617F or the immunogenically active peptide fragments of exon 9 mutant CALR or JAK2V617F of the invention in the vaccine composition may vary, depending on the particular application. However, a single dose of the peptide composition is preferably anywhere from about 10 μg to about 5000 μg, more preferably from about 50 μg to about 2500 μg such as about 100 μg to about 1000 μg. In particular, in embodiments of the invention where the individual to be treated is a human being, then a single dose may be in the range of 50 μg to 500 μg, for example in the range of 80 μg to 300 μg, such as in the range of 100 μg to 250 μg of exon 9 mutant CALR or JAK2V617F or said immunogenically active peptide fragment of exon 9 mutant CALR or JAK2V617F. Frequently, the vaccine compositions are administered repeatedly over time. For example the vaccine composition may be administered at least 2 times, preferably at least 5 times, more preferably at least 10 times, such as in the range of 10 to 20 times. The vaccine composition may also be administered continuously. Administration may be repeated at any useful frequency. Thus, for example the vaccine compositions may be administered once every week, such as once every two weeks, for example once every 3 weeks, such as once per month, for example once per two months, such as once per three months, for example once per half year, such as once per year. In particular, the vaccine compositions may be administered continuously. The frequency of administration may alter during said time. In one embodiment the vaccine compositions are administered continuously once per 1 to 3 months. Modes of administration include intradermal, subcutaneous and intravenous administration, implantation in the form of a time release formulation, etc. Any and all forms of administration known to the art are encompassed herein. Also any and all conventional dosage forms that are known in the art to be appropriate for formulating injectable immunogenically active peptide composition are encompassed, such as lyophilized forms and solutions, suspensions or emulsion forms containing, if required, conventional pharmaceutically acceptable carriers, diluents, preservatives, adjuvants, buffer components, etc.

The pharmaceutical compositions may be prepared and administered using any conventional protocol known by a person skilled in the art. In examples 3-5 non-limiting examples of preparation of a vaccine composition according to the invention is given as well as a non-limiting example of administration of such as a vaccine. It will be appreciated by the person skilled in the art that the protocol may be easily adapted to any of the vaccine compositions described herein. In a further embodiment of the invention, the pharmaceutical composition of the invention is useful for treating an individual suffering from a clinical condition characterized by expression of CALR or JAK2, such as myeloproliferative disorders, such as cancers.

The immunoprotective effect of the composition of the invention can be determined using several approaches known to those skilled in the art. A successful immune response may also be determined by the occurrence of DTH reactions after immunization and/or the detection of antibodies specifically recognizing the peptide(s) of the vaccine composition.

Vaccine compositions according to the invention may be administered to an individual in therapeutically effective amounts. The effective amount may vary according to a variety of factors such as the individual's condition, weight, sex and age. Other factors include the mode of administration.

The pharmaceutical compositions may be provided to the individual by a variety of routes such as subcutaneous, topical, oral and intramuscular. Administration of pharmaceutical compositions is accomplished orally or parenterally. Methods of parenteral delivery include topical, intra-arterial (directly to the tissue), intramuscular, subcutaneous, intramedullary, intrathecal, intraventricular, intravenous, intraperitoneal, or intranasal administration. The present invention also has the objective of providing suitable topical, oral, systemic and parenteral pharmaceutical formulations for use in the methods of prophylaxis and treatment with the vaccine composition.

For example, the vaccine compositions can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts. An effective but non-toxic amount of the vaccine, comprising any of the herein described compounds can be employed as a prophylactic or therapeutic agent. Also any and all conventional dosage forms that are known in the art to be appropriate for formulating injectable immunogenically active peptide composition are encompassed, such as lyophilized forms and solutions, suspensions or emulsion forms containing, if required, conventional pharmaceutically acceptable carriers, diluents, preservatives, adjuvants, buffer components, etc.

Preferred modes of administration of the vaccine composition according to the invention include, but are not limited to systemic administration, such as intravenous or subcutaneous administration, intradermal administration, intramuscular administration, intranasal administration, oral administration, rectal administration, vaginal administration, pulmonary administration and generally any form of mucosal administration. Furthermore, it is within the scope of the present invention that the means for any of the administration forms mentioned in the herein are included in the present invention.

A vaccine according to the present invention can be administered once, or any number of times such as two, three, four or five times. Administering the vaccine more than once has the effect of boosting the resulting immune response. The vaccine can further be boosted by administering the vaccine in a form or body part different from the previous administration. The booster shot is either a homologous or a heterologous booster shot. A homologous booster shot is a where the first and subsequent vaccinations comprise the same constructs and more specifically the same delivery vehicle especially the same viral vector. A heterologous booster shot is where identical constructs are comprised within different viral vectors.

Second Active Ingredient

It is an aspect of the present invention that the vaccine composition herein provided is used in combination with a second active ingredient. The administration of the vaccine composition and the second active ingredient may be sequential or combined. Examples of second active ingredients are given above for both cancers and infections. It is a further aspect that the vaccine composition may be used in combination with other therapy of relevance for the given clinical condition to be treated. Such therapy may include surgery, chemotherapy or gene therapy, immunostimulating substances or antibodies; a person skilled in the art is able to determine the appropriate combination treatment for a given scenario.

In some cases it will be appropriate to combine the treatment method of the invention with a further medical treatment such as chemotherapy, radiotherapy, treatment with immunostimulating substances, gene therapy, treatment with antibodies and/or antibiotics and treatment using dendritic cells.

Monitoring Immunization

In preferred embodiments, the pharmaceutical composition of the invention is a vaccine composition. It is therefore of interest, and an aspect of the present invention to monitor the immunization in an individual to whom the vaccine composition of the present invention is administered. The pharmaceutical composition may thus be an immunogenic composition or vaccine capable of eliciting an immune response to a cancer and/or infection. As used herein, the expression “immunogenic composition or vaccine” refers to a composition eliciting at least one type of immune response directed against cells expressing exon 9 mutant CALR or JAK2V617F such as malignant or cancer cells, APCs or DCs. Thus, such an immune response may be any of the following: A CTL response where CTLs are generated that are capable of recognizing the HLA/peptide complex presented on cell surfaces resulting in cell lysis, i.e. the vaccine elicits the production in the vaccinated subject of effector T-cells having a cytotoxic effect against the cancer cells; a B-cell response giving rise to the production of anti-cancer antibodies; and/or a DTH type of immune response. It is on object of the present invention to monitor the immunization of an individual by monitoring any of the above reactions subsequent to administering the composition of the present invention to said individual.

In one aspect the invention relates to methods of monitoring immunization, said method comprising the steps of

i) providing a blood sample from an individual

ii) providing exon 9 mutant CALR or JAK2V617F or a peptide fragment hereof, wherein said protein or peptide may be any of the proteins or peptides described herein

iii) determining whether said blood sample comprises antibodies or T-cells comprising T-cell receptors specifically binding the protein or peptide

iv) thereby determining whether an immune response to said protein or peptide has been raised in said individual.

The individual is preferably a human being, for example a human being that has been immunized with exon 9 mutant CALR or JAK2V617F or immunogenically active peptide fragments of exon 9 mutant CALR or JAK2V617F or a nucleic acid encoding said protein or peptide.

Kit of Parts

The invention also relates to a kit-of-parts comprising
any of the vaccine compositions described herein and/or
an exon 9 mutant CALR or JAK2V617F protein or functional homologue hereof and/or
any of the immunogenically active peptide fragments of an exon 9 mutant CALR or JAK2V617F, functional homologues hereof, and/or peptides derived here from as described herein and/or
any of the nucleic acids encoding the proteins of the above two bullet points and instructions on how to use the kit of parts.

The invention also relates to a kit-of-parts comprising
any of the vaccine compositions described herein and/or
an exon 9 mutant CALR or JAK2V617F protein or functional homologue hereof and/or
any of the immunogenically active peptide fragments of an exon 9 mutant CALR or JAK2V617F, functional homologues hereof, and/or peptides derived here from as described herein and/or
any of the nucleic acids encoding the proteins of the above two bullet points and a second active ingredient.

Preferably, the second active ingredient is chosen in correspondence with the clinical condition to be treated so that in the case where a cancer is to be treated the second active ingredient is chosen among e.g. chemotherapeutic agents as listed above. Likewise, if treating a microbial/viral infection, the second active ingredient is preferably an anti-biotic and/or an anti-viral agent.

The components of the kit-of-parts are preferably comprised in individual compositions, it is however within the scope of the present invention that the components of the kit-of-parts all are comprised within the same composition. The components of the kit-of-parts may thus be administered simultaneously or sequentially in any order.

Sequence listing

| SEQ ID NO | Description |
|---|---|
| SEQ ID NO: 1 | CALR Long 4 polypeptide |
| | RRMRRTRRKMRRKMSPARPRTSCREACLQGWTEA |
| SEQ ID NO: 2 | CALR Long 1 polypeptide |
| | RRMMRTKMRMRRMRRTRRKMRRKMSPARP |
| SEQ ID NO: 3 | CALR Long 2 polypeptide |
| | TRRKMRRKMSPARPRTSCREACLQGWTEA |
| SEQ ID NO: 4 | Human normal CALR polypeptide corresponding to exon 9 |
| | QDEEQRLKEEEEDKKRKEEEEAEDKEDDEDKDEDEEDE |
| | EDKEEDEEEDVPGQAKDEL |
| SEQ ID NO: 5 | Wild type JAK2 sequence |
| | >sp\|O60674\|JAK2_HUMAN Tyrosine-protein kinase JAK2 |
| | OS = Homo sapiens GN = JAK2 PE = 1 SV = 2 |
| | MGMACLTMTEMEGTSTSSIYQNGDISGNANSMKQIDPVL |
| | QVYLYHSLGKSEADYLTFPSGEYVAEEICIAASKACGITPV |
| | YHNMFALMSETERIWYPPNHVFHIDESTRHNVLYRIRFYF |
| | PRWYCSGSNRAYRHGISRGAEAPLLDDFVMSYLFAQWR |
| | HDFVHGWIKVPVTHETQEECLGMAVLDMMRIAKENDQT |
| | PLAIYNSISYKTFLPKCIRAKIQDYHILTRKRIRYRFRRFIQQ |
| | FSQCKATARNLKLKYLINLETLQSAFYTEKFEVKEPGSGP |
| | SGEEIFATIIITGNGGIQWSRGKHKESETLTEQDLQLYCDF |
| | PNIIDVSIKQANQEGSNESRVVTIHKQDGKNLEIELSSLR |
| | EALSFVSLIDGYYRLTADAHHYLCKEVAPPAVLENIQSNC |
| | HGPISMDFAISKLKKAGNQTGLYVLRCSPKDFNKYFLTFA |
| | VERENVIEYKHCLITKNENEEYNLSGTKKNFSSLKDLLNC |
| | YQMETVRSDNIIFQFTKCCPPKPKDKSNLLVFRTNGVSD |
| | VPTSPTLQRPTHMNQMVFHKIRNEDLIFNESLGQGTFTKI |
| | FKGVRREVGDYGQLHETEVLLKVLDKAHRNYSESFFEAA |
| | SMMSKLSHKHLVLNYGVCVCGDENILVQEFVKFGSLDTY |
| | LKKNKNCINILWKLEVAKQLAWAMHFLEENTLIHGNVCAK |
| | NILLIREEDRKTGNPPFIKLSDPGISITVLPKDILQERIPWVP |
| | PECIENPKNLNLATDKWSFGTTLWEICSGGDKPLSALDS |
| | QRKLQFYEDRHQLPAPKWAELANLINNCMDYEPDFRPS |
| | FRAIIRDLNSLFTPDYELLTENDMLPNMRIGALGFSGAFE |
| | DRDPTQFEERHLKFLQQLGKGNFGSVEMCRYDPLQDNT |
| | GEVVAVKKLQHSTEEHLRDFEREIEILKSLQHDNIVKYKG |
| | VCYSAGRRNLKLIMEYLPYGSLRDYLQKHKERIDHIKLLQ |
| | YTSQICKGMEYLGTKRYIHRDLATRNILVENENRVKIGDF |
| | GLTKVLPQDKEYYKVKEPGESPIFWYAPESLTESKFSVA |
| | SDVWSFGVVLYELFTYIEKSKSPPAEFMRMIGNDKQGQM |
| | IVFHLIELLKNNGRLPRPDGCPDEIYMIMTECWNNNVNQR |
| | PSFRDLALRVDQIRDNMAG |
| SEQ ID NO: 6 | Mutant JAK2V617F |
| | MGMACLTMTEMEGTSTSSIYQNGDISGNANSMKQIDPVL |
| | QVYLYHSLGKSEADYLTFPSGEYVAEEICIAASKACGITPV |
| | YHNMFALMSETERIWYPPNHVFHIDESTRHNVLYRIRFYF |
| | PRWYCSGSNRAYRHGISRGAEAPLLDDFVMSYLFAQWR |
| | HDFVHGWIKVPVTHETQEECLGMAVLDMMRIAKENDQT |
| | PLAIYNSISYKTFLPKCIRAKIQDYHILTRKRIRYRFRRFIQQ |
| | FSQCKATARNLKLKYLINLETLQSAFYTEKFEVKEPGSGP |
| | SGEEIFATIIITGNGGIQWSRGKHKESETLTEQDLQLYCDF |
| | PNIIDVSIKQANQEGSNESRVVTIHKQDGKNLEIELSSLR |
| | EALSFVSLIDGYYRLTADAHHYLCKEVAPPAVLENIQSNC |
| | HGPISMDFAISKLKKAGNQTGLYVLRCSPKDFNKYFLTFA |
| | VERENVIEYKHCLITKNENEEYNLSGTKKNFSSLKDLLNC |
| | YQMETVRSDNIIFQFTKCCPPKPKDKSNLLVFRTNGVSD |
| | VPTSPTLQRPTHMNQMVFHKIRNEDLIFNESLGQGTFTKI |
| | FKGVRREVGDYGQLHETEVLLKVLDKAHRNYSESFFEAA |
| | SMMSKLSHKHLVLNYGVCFCGDENILVQEFVKFGSLDTY |
| | LKKNKNCINILWKLEVAKQLAWAMHFLEENTLIHGNVCAK |
| | NILLIREEDRKTGNPPFIKLSDPGISITVLPKDILQERIPWVP |
| | PECIENPKNLNLATDKWSFGTTLWEICSGGDKPLSALDS |
| | QRKLQFYEDRHQLPAPKWAELANLINNCMDYEPDFRPS |
| | FRAIIRDLNSLFTPDYELLTENDMLPNMRIGALGFSGAFE |
| | DRDPTQFEERHLKFLQQLGKGNFGSVEMCRYDPLQDNT |
| | GEVVAVKKLQHSTEEHLRDFEREIEILKSLQHDNIVKYKG |
| | VCYSAGRRNLKLIMEYLPYGSLRDYLQKHKERIDHIKLLQ |
| | YTSQICKGMEYLGTKRYIHRDLATRNILVENENRVKIGDF |
| | GLTKVLPQDKEYYKVKEPGESPIFWYAPESLTESKFSVA |
| | SDVWSFGVVLYELFTYIEKSKSPPAEFMRMIGNDKQGQM |
| | IVFHLIELLKNNGRLPRPDGCPDEIYMIMTECWNNNVNQR |
| | PSFRDLALRVDQIRDNMAG |
| SEQ ID NO: 7 | JAK2 polypeptide (JAK2₆₁₀₋₆₁₈) |
| | VLNYGVCFC |
| SEQ ID NO: 8 | Scrambled peptide |
| | MRRTMMMMMPRRRRRRKRRSKTRAPRMRK |
| SEQ ID NO: 9 | Human wild-type CALR |
| | >sp\|P27797\|CALR HUMAN Calreticulin OS = Homo |
| | sapiens GN = CALR PE = 1 SV = 1 |
| | MLLSVPLLLGLLGLAVAEPAVYFKEQFLDGDGWTSRWIE |
| | SKHKSDFGKFVLSSGKFYGDEEKDKGLQTSQDARFYAL |
| | SASFEPFSNKGQTLVVQFTVKHEQNIDCGGGYVKLFPNS |
| | LDQTDMHGDSEYNIMFGPDICGPGTKKVHVIFNYKGKNV |
| | LINKDIRCKDDEFTHLYTLIVRPDNTYEVKIDNSQVESGSL |
| | EDDWDFLPPKKIKDPDASKPEDWDERAKIDDPTDSKPED |
| | WDKPEHIPDPDAKKPEDWDEEMDGEWEPPVIQNPEYKG |
| | EWKPRQIDNPDYKGTWIHPEIDNPEYSPDPSIYAYDNFG |
| | VLGLDLWQVKSGTIFDNFLITNDEAYAEEFGNETWGVTK |
| | AAEKQMKDKQDEEQRLKEEEEDKKRKEEEEAEDKEDDE |
| | DKDEDEEDEEDKEEDEEEDVPGQAKDEL |
| SEQ ID NO: 10 | Amino acid sequence of the full-length exon 9 mutant |
| | CALR L367fs*46 |
| | MLLSVPLLLGLLGLAVAEPAVYFKEQFLDGDGWTSRWIE |
| | SKHKSDFGKFVLSSGKFYGDEEKDKGLQTSQDARFYAL |
| | SASFEPFSNKGQTLVVQFTVKHEQNIDCGGGYVKLFPNS |
| | LDQTDMHGDSEYNIMFGPDICGPGTKKVHVIFNYKGKNV |
| | LINKDIRCKDDEFTHLYTLIVRPDNTYEVKIDNSQVESGSL |
| | EDDWDFLPPKKIKDPDASKPEDWDERAKIDDPTDSKPED |
| | WDKPEHIPDPDAKKPEDWDEEMDGEWEPPVIQNPEYKG |
| | EWKPRQIDNPDYKGTWIHPEIDNPEYSPDPSIYAYDNFG |
| | VLGLDLWQVKSGTIFDNFLITNDEAYAEEFGNETWGVTK |
| | AAEKQMKDKQDEEQRTRRMMRTKMRMRRMRRTRRKM |
| | RRKMSPARPRTSCREACLQGWTEA |
| SEQ ID NO: 11 | Sequence overlap between SEQ ID NO: 2 and SEQ ID NO: |
| | 3 |
| | RRMMRTKMRM |
| SEQ ID NO: 12 | CALR exon 9 mutant type 2 |
| | NCRRMMRTKMRMRRMRRTRRKMRRKMSPARPRTSCR |
| | EACLQGWTE |
| SEQ ID NO: 13 | Mutant human CALR polypeptide corresponding to exon 9 |
| | of L367fs*46 mutant |
| | QDEEQRTRRMMRTKMRMRRMRRTRRKMRRKMSPARP |
| | RTSCREACLQGWTEA |
| SEQ ID NO: 14 | Overlapping sequences of the C-terminus of exon 9 CALR |
| | type 1 and type 2 mutations: |
| | RRMMRTKMRMRRMRRTRRKMRRKMSPARPRTSCREA |
| | CLQGWTE |
| SEQ ID NO: 15 | CALR Long 3 polypeptide |
| | KMRMRRMRRTRRKMRRKMSP |
| SEQ ID NO: 16 | Consensus sequence of type 1 and type 2 CALR exon 9 |
| | mutations |
| | RMRRMRRTRRKMRRKMSPARPRTSCREACLQGWTEA |
| SEQ ID NO: 17 | CALR Long 5 polypeptide |
| | RRMMRTKMRMRRMRRTRRKMRRKMSPARPRTSCREA |
| | CLQGWTEA |
| SEQ ID NO: 18 | Scrambled peptide 2 |
| | TSMMRRRRRRKRRKMMKRM |
| SEQ ID NO: 19 | CALR siRNA sense sequence |
| | GGAGCAGUUUCUGGACGGATT |
| SEQ ID NO: 20 | CALR siRNA antisense sequence |
| | UCCGUCCAGAAACUGCUCCTT |
| SEQ ID NO: 21 | JAK2V617F duplex 1 sense sequence |
| | GAGUAUGUUUCUGUGGAGATT |
| SEQ ID NO: 22 | JAK2V617F duplex 1 antisense sequence |
| | UCUCCACAGAAACAUACUCTT |
| SEQ ID NO: 23 | JAK2V617F duplex 2 sense sequence |
| | GGAGUAUGUUUCUGUGGAGTT |
| SEQ ID NO: 24 | JAK2V617F duplex 2 antisense sequence |
| | CUCCACAGAAACAUACUCCTT |
| SEQ ID NO: 25 | JAK201wt, JAK201 wild type epitope |
| | VLNYGVCVC |
| SEQ ID NO: 26 | HLA-A2 high affinity-binding epitope HIV-1 |
| | ILKEPVHGV |

### EXAMPLES

The invention is further illustrated by the following examples, which should however not be construed as limiting for the invention.

### Example 1—CALR

Introduction

This study aimed at describing the spontaneous T-cell responses against the CALR exon 9 mutations in patients with CALR mutated chronic myeloproliferative neoplasms (MPN). These T-cell responses were directed against two overlapping peptides that spanned the mutated CALR C-terminus.

Responses of MPN Patients to CALR Long1 and CALR Long2 Peptides

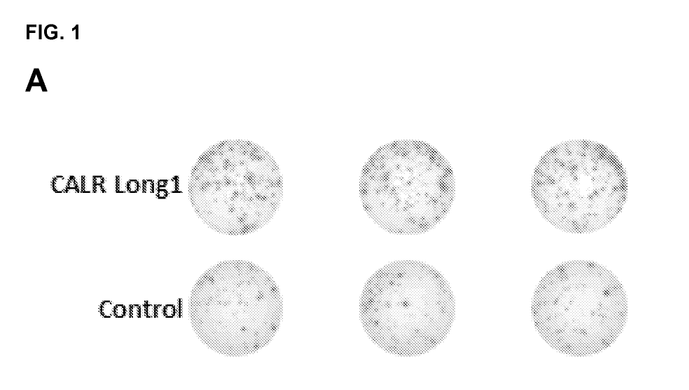

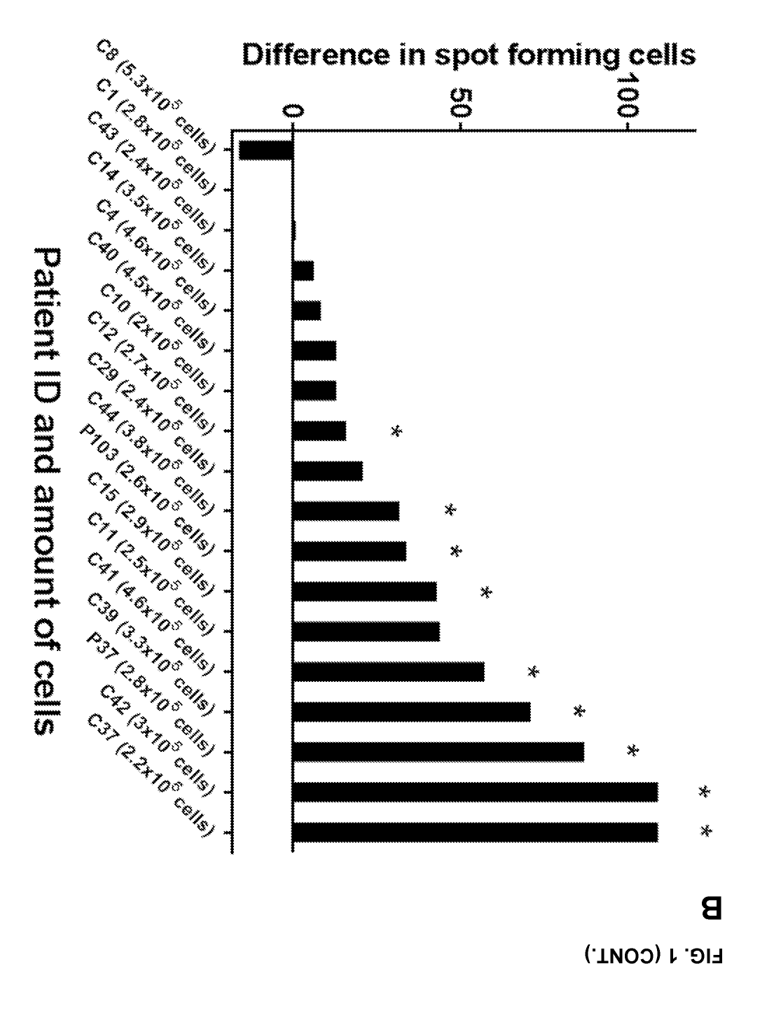

The study was approved by the local ethics committee, and all patients signed informed consent, in accordance with the Helsinki Declaration before study participation. We included 31 CALRmut MPN patients with the following diagnoses: ET (n=13), PMF (n=12), post-ET MF (n=4), or prefibrotic MF (n=2). Peripheral blood mononuclear cells (PBMC) were isolated with Lymphoprep and frozen in fetal calf serum with 10% dimethyl sulfoxide. First, we scrutinized PBMC from two MPN patients for the presence of spontaneous T-cell responses against two CALR derived peptides, CALR Long1 (RRMMRTKMRMRRMRRTRRKMRRKMSPARP, SEQ ID NO: 2) and CALR Long2 (TRRKMRRKMSPARPRTSCREACLQGWTEA, SEQ ID NO: 3) using the highly sensitive and solid interferon gamma (IFN-γ) Enzyme-Linked ImmunoSPOT (ELISPOT) secretion assays (FIGS. 1A and 1C). The ELISPOT has proven to be the central assay in studies focusing on identification on novel tumor antigens by the characterization of novel tumor antigen responses in PBMC from cancer patients. Thus, this assay has previously been utilized for the identification of novel tumor antigens based on spontaneous immunity in cancer patients (Andersen et al., 2005).

The ELISPOT assay is based on the detection of antigen induced release of cytokines—most often IFN-γ—by single T-cells upon triggering of its T-cell receptor. Reactivity of a single T-cell can be detected and quantified via binding of the respective cytokine on special nitrocellulose filter plates. When a T-cell recognizes the peptide epitope examined, the T-cell releases cytokines that are detected by a colorimetric reaction using an enzyme conjugated to a secondary cytokine specific antibody. The reaction product is visible as a spot. Ideally, each spot represents the cytokines secreted by a single activated cell. ELISPOTs were performed and analyzed as described previously (Munir et al., 2013). PBMCs were tested in different concentrations to ensure an optimal response. The distribution-free resampling method, described by Moodie et al., was used for statistical analyses and a p-value ≤0.05 was deemed a T-cell response (Moodie et al., 2010).

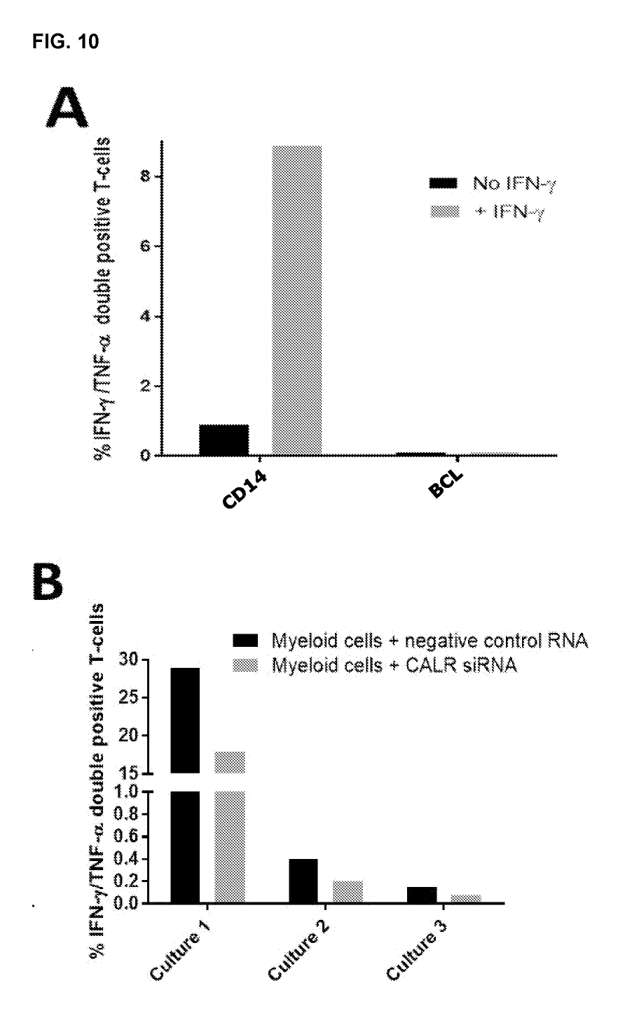

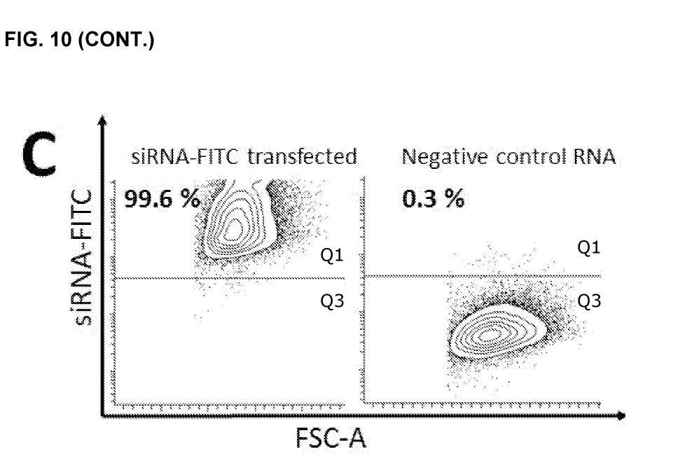

After the promising responses in the first two patients we analyzed PBMC from additional 29 CALR-mutated MPN patients. Some of the patients' PBMC showed low viability; consequently, we were able to analyze responses against CALR Long1 in 18 patients and against CALR Long2 in 24 patients. Of the 24 evaluable patients, 10 had ET and 14 had MF; the latter group comprised 11 patients with PMF, 2 with post-ET MF, and 1 with prefibrotic MF. Analyses showed that 9 patients (50%) responded to CALR Long1 (FIG. 1C) and 10 patients (42%) to CALR Long2 (FIG. 1D); among these patients, 6 (32%) responded to both CALR Long1 and Long2. To confirm these results, we performed an additional ELISPOT in samples from 5 of the 9 patients that showed responses against CALR Long1, and the response was confirmed in 4 patients. Likewise, we performed a supplementary ELISPOT in 5 of the 10 patients that showed responses against CALR Long2, and we confirmed the response in 4 patients. Furthermore, we examined PBMC from two patients in a tumor necrosis factor alpha (TNF-α) ELISPOT. Interestingly, a significant TNF-α response was detected in one of these patients (data not shown).

The immune system interacts closely with tumors over the entire process of disease development. Hence, while patients with non-advanced cancer can maintain an immune response against the cancer, in advanced stages the cancer manages to evade the immune response (Dunn et al., 2002). ET and MF are different stages of MPN in the biological continuum from early (ET) to the advanced cancer stage (MF) (Hasselbalch et al., 2009). Indeed, we observed more frequent responses in patients with ET, where 8 of 10 (80%) showed a response, compared to patients with MF, where only 5 of 14 (36%) mounted a response. This difference was statistically significant with p=0.047 by Fisher's exact test.

The Response does not Depend on the CALR Mutation Type

Several publications have shown that the different types of CALR mutations have different impact on disease phenotype and prognosis (Tefferi, Wassie et al., 2014; Cabagnols et al., 2015; Tefferi, Lasho et al., 2014). Accordingly, one might expect askewness in mutation types among patients that respond to the CALR peptides. Of our 24 evaluable patients, 11 (46%) had a type 1 mutation; of these, 5 showed an IFN-γ response (45%). Nine patients (38%) had a type 2 mutation; of these, 6 had an IFN-γ response (67%). Four patients (17%) had neither a type 1 nor a type 2 mutation and of these, 2 showed an IFN-γ response (50%). The difference in response between patients with type 1 and type 2 mutations did not reach statistical significance (p=0.41), which suggests that the mutation type does not influence the capability of the immune system to react to the CALR mutation.

T-Cell Response Analysis

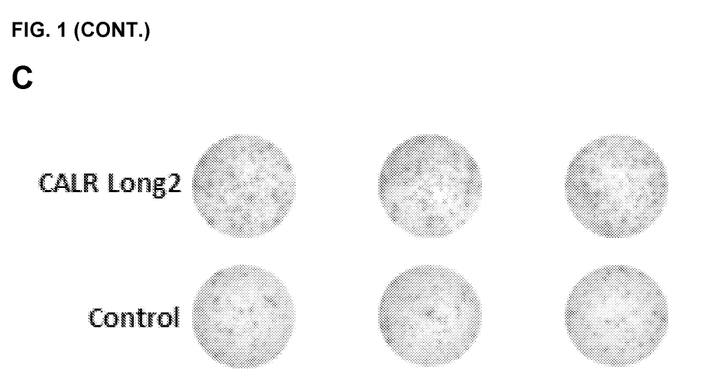

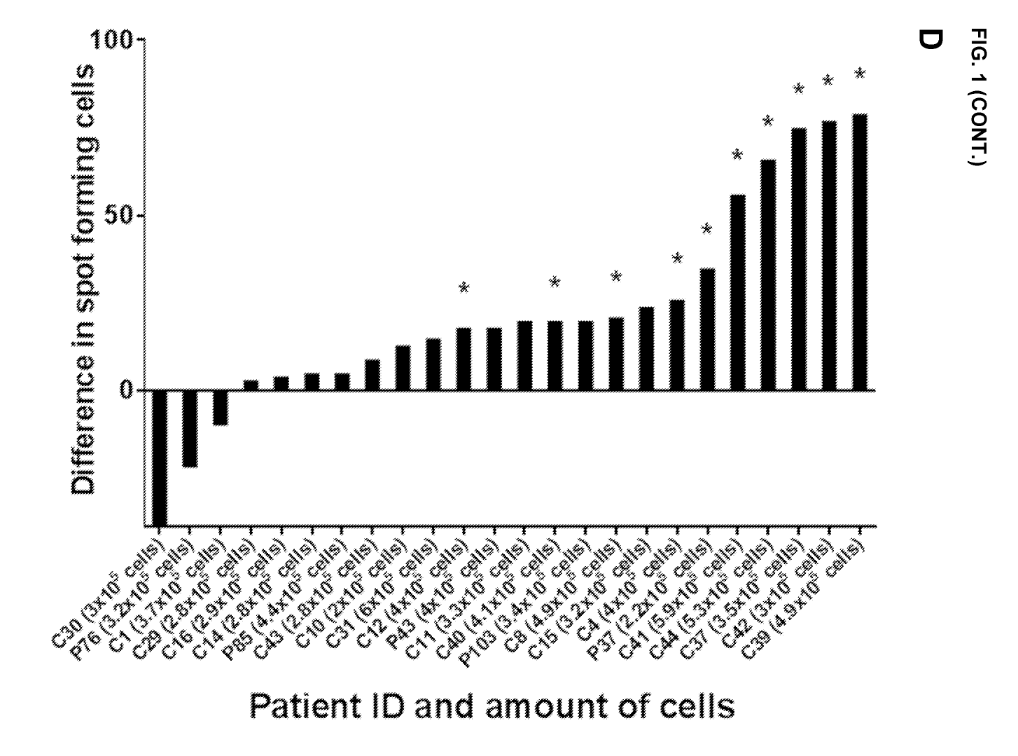

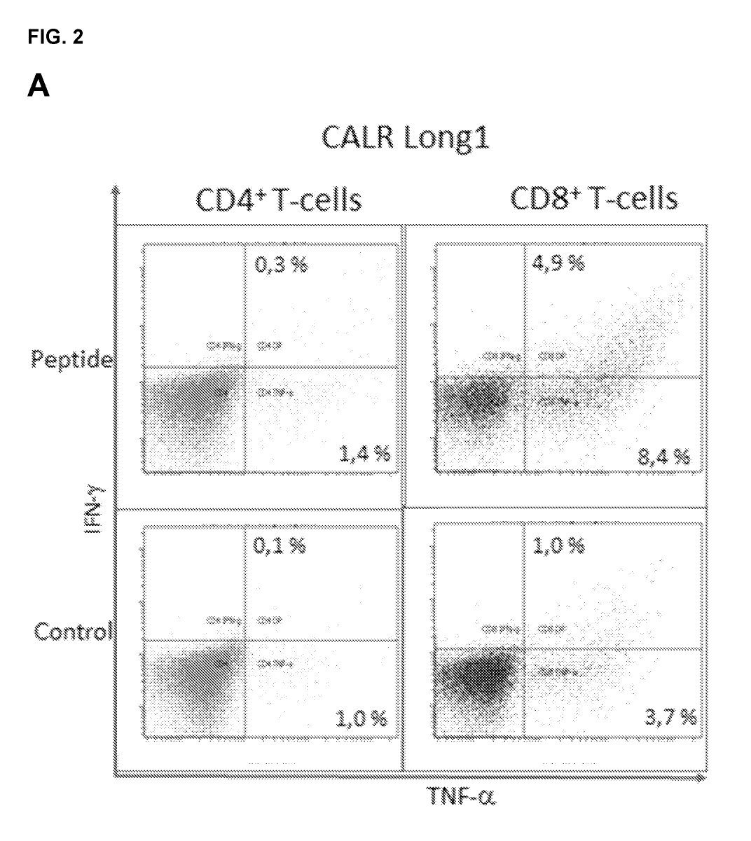

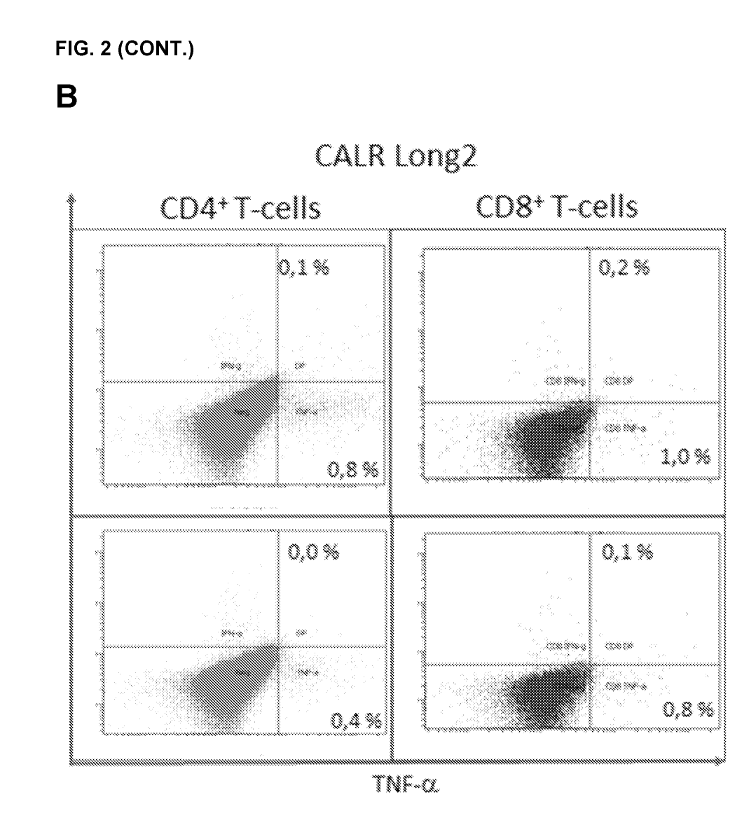

Next, we chose to analyze PBMC from four patients with a response against CALR Long1 and CALR Long2 peptides using intracellular cytokine staining. Although this method is less sensitive than ELISPOT, it allows elucidating which immune cells secrete the cytokine identified in ELISPOT. Hereby, we could determine whether the reacting cells were T-cells and, if so, if the responding cells were CD4 T-cells or CD8 T-cells. In short, cells were stimulated with CALR Long1 or with a scrambled peptide (MRRTMMMMMPRRRRRRKRRSKTRAPRMRK) as control. For surface markers we employed 4 μl NIR, 10 μl CD4 PerCP, 2 μl CD8 Pacific Blue, and 10 μl CD3 FITC and for intracellular staining we used 2 μl anti-TNF-α and anti IFN-γ antibodies conjugated with either PE-Cy7 or APC. Washing, permeabilization and staining procedures followed previously described methods (Munir, Andersen et al., 2013). The results from the intracellular cytokine staining demonstrated a response in 2 of the 4 patients analyzed. One patient (C42) responded to CALR Long1 with a profound CD8 T-cell response and a more modest CD4 T-cell response (FIG. 2A). The other patient (C39) responded to both CALR Long1 (data not shown) and CALR Long2 with a CD4 T-cell response (data not shown). To confirm that the responses identified indeed were T-cell responses against CALR Long1, we generated dendritic cells from patient C42 using previously described methods. Autologous PBMC were stimulated three times with the dendritic cells, which had been pulsed with CALR Long1, and we hereby generated a CALR Long1 specific T-cell culture. Using intracellular cytokine staining we demonstrated that the T-cell culture mounted a strong CD4 T-cell response against CALR Long1 and a somewhat weaker CD8 T-cell response (FIG. 2B). These data show that the immune responses identified in the ELISPOT assays are, indeed, T-cell mediated responses against mutated CALR peptides. The frequent detection of T-cell responses indicate, that the CALR mutations encode highly immunogenic cancer antigens, which represent ideal targets for anti-cancer immune therapy.

In general, a major limitation in targeting mutant antigens in cancer immunotherapy has been that different patients display different antigens. This problem is not likely to limit approaches that target the CALR exon 9 mutations, because all CALR mutations share the 36 amino acid consensus sequence. This short sequence is the optimal size for designing an agent that can identify antigenic peptides. In addition, the two most frequent mutation types (types 1 and 2) are found in more than 80% of the patients, and these types share an additional 10-amino-acid consensus sequence (SEQ ID NO: 11). Here, we describe the immunogenic potential of two CALR consensus sequences; one was a consensus for both the type 1 and type 2 mutations (CALR Long1) and the other was a pan-CALR consensus for mutations in the 36-amino-acid terminal sequence (CALR Long2). Recently, it has been shown that patients with CALR mutated MPN may harbor several types of CALR mutations (Jeromin et al., 2016) and therefore it is of utmost importance, that the immune system mounts an immune response against the 34-amino-acid terminal sequence, hereby targeting all the CALR mutations. Since we describe that the immune system targets both the type 1 and type 2 mutations (CALR Long1) and the 36-amino-acid consensus sequence (CALR Long2), the immune system might actually both eliminate the dominant CALR mutated clone and additional subclones. Thus, our results suggest that CALR exon 9 mutations are viable targets for cancer immune therapies; for example as targets for vaccines or adoptive cell therapies.

### Example 2

Patient

Patient peripheral blood mononuclear cells (PBMCs) were isolated using Lymphoprep (Axis Shield, Oslo, Norway), and were frozen in fetal calf serum with 10% dimethyl sulfoxide (Sigma-Aldrich). The patient provided signed informed consent prior to study participation in accordance with the Helsinki Declaration, and this study was approved by the local ethics committee. To generate CALRLong1 specific-T-cell culture, we chose to work with cells from a patient with a strong CD8⁺ T-cell response and a more modest CD4⁺ T-cell response against the CALRLong1 epitope. The patient is a 74-year old male Caucasian with a CALR type 33 mutation consisting of a 5 bp insertion. He was diagnosed with ET 18 years prior to his inclusion in the project. Fourteen years after his diagnosis, a new bone marrow biopsy revealed that the patient had progressed to post-essential thrombocythemia myelofibrosis. At the time of blood sampling for this project the patient was treated with anagrelide.

Peptides

We choose to work with the following peptides, that were either provided by KJ Ross-Petersen, Klampenborg, Denmark or Schafer-N, Copenhagen, Denmark: CALRLong1 (RRMMRTKMRMRRMRRTRRKMRRKMSPARP, SEQ ID NO: 2), CALRLong2 (TRRKMRRKMSPARPRTSCREACLQGWTEA, SEQ ID NO: 3), CALRLong3 (KMRMRRMRRTRRKMRRKMSP, SEQ ID NO: 15), CALRLong4 (RRMRRTRRKMRRKMSPARPRTSCREACLQGWTEA, SEQ ID NO: 1) and CALRLong5 (RRMMRTKMRMRRMRRTRRKMRRKMSPARPRTSCREACLQGWTEA, SEQ ID NO: 17). As negative controls, we used one of two scrambled peptides:

(SEQ ID NO: 8)
MRRTMMMMMPRRRRRRKRRSKTRAPRMRK
or
(SEQ ID NO: 18)
TSMMRRRRRRKRRKMMKRM.

Generation of a T-Cell Culture Specific for CALRmut Epitopes

Fresh PBMCs were first enriched for CD14₊ cells using MACS CD14 MicroBeads (Miltenyi Biotech GmbH, Bergisch Gladbach, Germany). This was considered day 0. The resulting monocyte-depleted cell culture was termed peripheral blood lymphocytes (PBL). The enriched cells were cultured using CellGro (CellGenix GmbH, Freiburg, Germany), and stimulated with GM-CSF (1000 U/ml) and IL-4 (250 U/ml) (both PeproTech, Rocky Hill, N.J., USA). The next day, to generate fast dendritic cells (fastDC), we treated half of the enriched cells with a maturation cocktail comprising IFN-γ (1000 U/ml) (PeproTech, Rocky Hill, N.J., USA), PolyI:C (20 μg/ml) (Sigma-Aldrich, St. Louis, Mo., USA) and CALRLong1 peptide. PBL were cultured overnight in X-VIVO medium (Lonza Group Ltd., Basel, Switzerland) and then stimulated with half of the fastDC. The other half of the fastDC were frozen for later use.

The other half of the enriched cells were cultured until day 7, at which time these cells were treated with the same maturation cocktail as above, to generate mature dendritic cells (mDC). The next day, these mDC were used to stimulate PBLs. On day 9, IL-2 (120 U/mL) (Novartis, Basel, Switzerland) and IL-7 (40 U/ml) (PeproTech, Rocky Hill, N.J., USA) was added. On day 14, we thawed the fastDC that had been frozen on day 2, and used these cells to stimulate the PBL. On day 16 and day 24 we added, IL-2 and IL-7 in the concentrations mentioned above.

IFN-γ Enrichment and CD4⁺ T-cell Cloning

At day 38 of the T-cell culture, we stimulated the T-cells with CALRLong1 peptide for 4 h and next isolated IFN-γ secreting cells using the MACS IFN-γ secretion assay. The isolated cells were expanded using a rapid expansion protocol. The vast majority of cells that reacted to CALRLong1 peptide stimulation were CD4⁺ T-cells; thus, we isolated CD4⁺ T cells from the REP culture using the EasySep Human CD4 isolation Kit (StemCell Technologies, Inc., Vancouver, BC, Canada). The enriched CD4⁺ T-cells were then cloned by limiting dilution, and growing T-cell clones were rapidly expanded using feeder cells and high dose IL-2 (6000 u/ml). T-cell receptor clonotyping of the cloned cells was performed by denaturing gradient gel electrophoresis as previously described.

Target Cells

As target cells, we used autologous CD14⁺ monocytes and B-cells (BCL). As described above, CD14⁺ monocytes were isolated using the MACS CD14 MicroBead kit. An autologous B-cell line was generated using EBV transfection of donor BCL at the Health Protection Agency Culture Collections, Salisbury, UK.

Intracellular Cytokine Staining

Frozen T-cells were thawed one day prior to assaying, and in experiments examining CD107a expression, a CD107a-PE antibody was added to cells directly after addition of peptide or target cells. After one hour of stimulation, protein-transport inhibitor Brefeldin A (BD Biosciences, San Jose, Calif., USA) was added to the cells which were then stimulated for four h. Cells were stained with CD3-APC-H7, CD4-FITC, CD8-PerCP, IFN-γ-APC, TNF-α-BV421 and Fixable Viability Stain 510 (BD Biosciences, San Jose, Calif., USA), permeabilized and washed with Permeabilization Reagent/Diluent and fixation-permeabilization buffer (AH Diagnostics, Aarhus, Denmark). Cells were acquired with a FACS Canto II (BD Biosciences) and analyzed using FACSDiva software. All effector cell stimulations were run in triplicate wells if not otherwise specified.

siRNA Mediated CALR Silencing

Silencer siRNA duplex for targeted silencing of CALR with a 3′-end dT overhang in the antisense strand were obtained from Invitrogen (Invitrogen, Paisley, UK). The CALR siRNA duplex consisted of the sense sequence GGAGCAGUUUCUGGACGGATT (SEQ ID NO: 19) and the antisense sequence UCCGUCCAGAAACUGCUCCTT (SEQ ID NO: 20). Silencer siRNA duplexes were resuspended in RNase-free water to a concentration of 25 μM and subsequently stored at −20° C.

Autologous CD14⁺ monocytes were cultured with CellGro, GM-CSF (1000 U/ml) and IL-4 (250 U/mL) and transfected with either CALR siRNA or negative-control siRNA (Invitrogen) the next day, using electroporation parameters as described previously (Met et al., 2011). In brief, monocytes were washed twice, suspended in Opti-MEM medium (Invitrogen) and adjusted to a final cell density of 10⁷ cells/ml. The cell suspension (200 μl) was preincubated for 5 min on ice and subsequently, together with 5 μl of CALR siRNA or 10 μl of negative control siRNA transferred to a 2-mm gap electroporation and pulsed using a BTX 830 square-wave electroporator (Harvard Apparatus, Holliston Mass., USA). Electroporation settings were adjusted to a single pulse, 250 V, 2 ms. Immediately after electroporation, monocytes were transferred to prewarmed CellGro with 1000 U/ml GM-CSF and 250 U/ml IL-4. Electroporated monocytes were incubated and used for experimental analysis as specified in the text. Flow cytometric analysis of monocytes transfected with FITC siRNA (BD Biosciences, San Jose, Calif., USA) was used to ensure proper transfection efficiency.

Results

CALRLong1-Specific T-Cells Recognizes Several CALRmut Epitopes

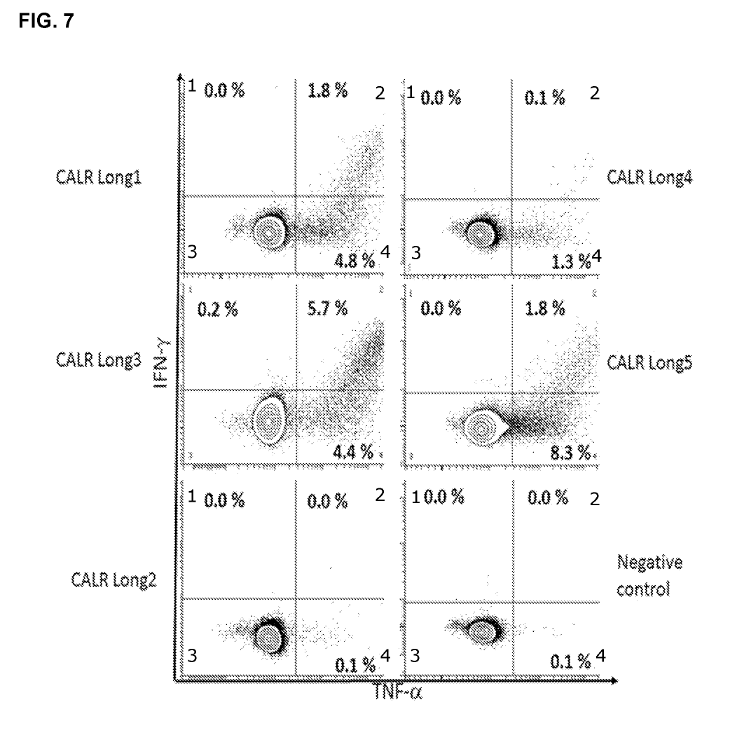

We established a CALRLong1-specific T-cell culture from a CALRmut patient with a strong CD8⁺ T-cell response against CALRLong1 peptide. PBL were stimulated three times with autologous dendritic cells, and T-cells were analyzed using intracellular cytokine staining (ICS). Surprisingly, the CD8⁺ T cells showed no response against CALRLong1 (data not shown). However, CD4⁺ T cells responded to CALRLong1 (FIG. 7). Next, we investigated whether these CALRLong1-specific T-cells were able to recognize other epitopes in the CALRmut C-terminus. The T-cells reacted upon stimulation with both CALRLong3, CALRLong4 and CALRLong5 (FIG. 7), whereas, the T-cells did not respond upon stimulation with CALRLong2 (FIG. 7) suggesting that the response was against the beginning of the mutated sequence. Since CALRLong4 peptide comprises the shared 36 amino acid C-terminus found in all CALRmut patients, we tested PBMCs from the donor for a spontaneous ex-vivo ELISPOT response in a 48 hour assay. Despite a high background, we found a significant response to CALRLong4 peptide in that patient as defined by the rules supplied by Moodie et al. (2010) (not shown).

Cloning of Highly Enriched CALRLong1-Specific CD4⁺ T-Cells

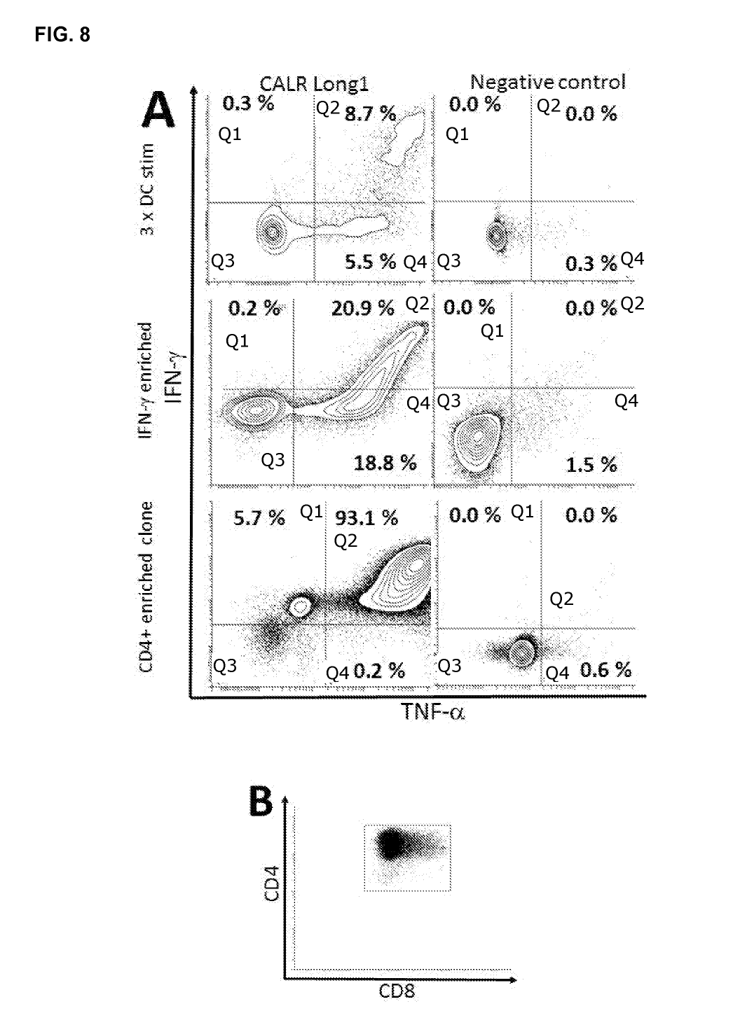

To obtain a CALRLong1-specific CD4⁺ T-cell population of higher purity, we enriched the CALRLong1-specific T cells using the MACS IFN-γ secretion assay. This increased the proportion of CALRLong1 specific CD4⁺ T-cells (FIG. 8A). Since almost 40% of the CD4⁺ T cells responded against CALRLong1 peptide, we next enriched the CD4⁺ T cells in the culture and established CALRLong1-specific CD4⁺ T-cell clones. We used ICS to demonstrate that one of the CD4⁺ T-cell cultures was 100% specific for CALRLong1 (FIG. 8A). Immune phenotyping by flow cytometry revealed that the culture was a pure CD4⁺ T-cell culture (FIG. 8B), and T-cell receptor clonotyping of the CD4⁺ CALRLong1-specific T-cells confirmed that the T-cells were clones (data not shown).

CALRLong1-Specific T Cells are Activated Upon Stimulation with Autologous CALRmut Cells

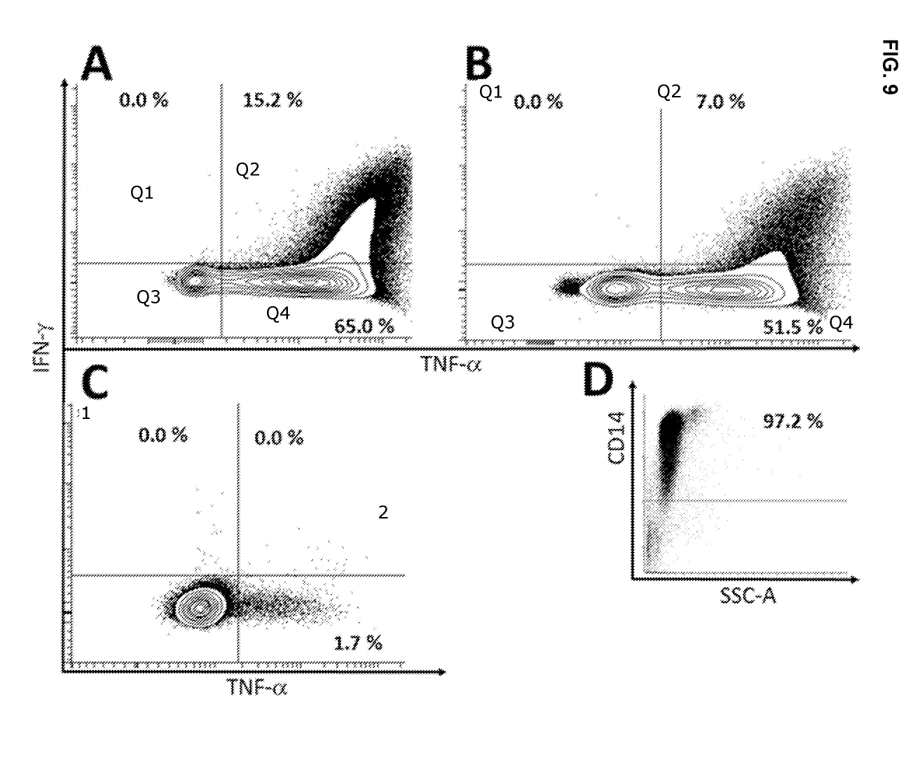

To assess whether CALR exon 9 mutations might be targeted by the immune system, it is crucial to demonstrate that the CALRLong1-specific T cells recognize and are activated upon stimulation with CALRmut cells. As target cells, we used CD14⁺ monocytes with CALRmut allelic burden of 71% and autologous EBV-transformed immortalized B-cells with a CALRmut allelic burden of 0% determined by PCR analysis. The CALRLong1-specific T cells were activated upon stimulation with autologous CD14⁺ monocytes, with approximately 80% of T-cells secreting cytokine at an effector:target ratio of 1:1 (FIG. 9A). Stimulation with B-cells at the same effector:target ratio resulted in cytokine secretion from 1.7% of the T-cells (FIG. 9C). Moreover, fewer T cells were activated when the effector:target ratio was 3:1. (FIG. 9B). Purity analysis of the target cells revealed high enrichment of CD14⁺ monocytes (FIG. 9D).

T-Cell Activation by CALRmut Target Cells is Dependent Upon Antigen Presentation by Target Cells

To prove that T-cell recognition and activation relied on the presence of mutant CALR antigen, we decided to stimulate target cells with IFN-γ (300 U/ml) for 24 h prior to assessment to enhance antigen presentation. Stimulation of CALRmut monocytes with IFN-γ significantly enhanced T-cell activation, however no change in activation was seen in T-cells stimulated with IFN-γ treated B-cells (FIG. 10A). Next, we transfected autologous CD14⁺ sorted myeloid cells with CALR siRNA as described in materials and methods. Stimulating the T-cells with siRNA-transfected target cells 48 h after transfection led to a nearly 50% decrease in the proportion of IFN-γ/TNF-α double-positive T cells compared to negative controls (FIG. 10B). Flow cytometric analysis of myeloid cells transfected with FITC-conjugated siRNA (FIG. 10C) showed proper transfection efficiency.

The CALRmut-Specific Response is HLA II Restricted with HLA-DR as the Restriction Element

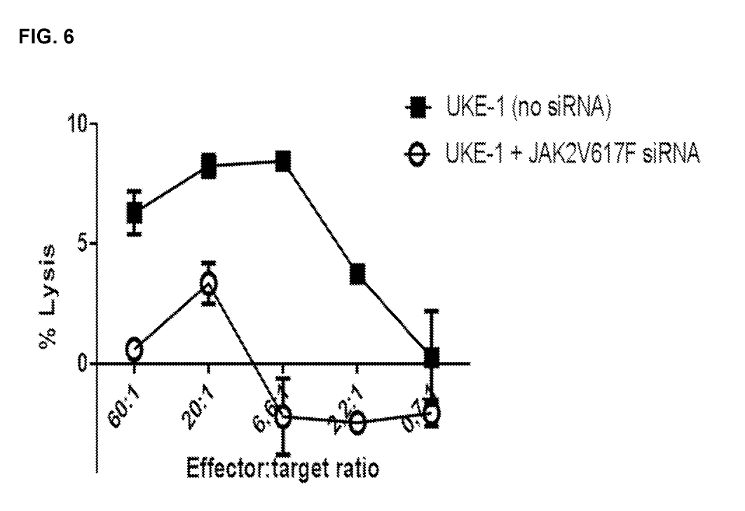

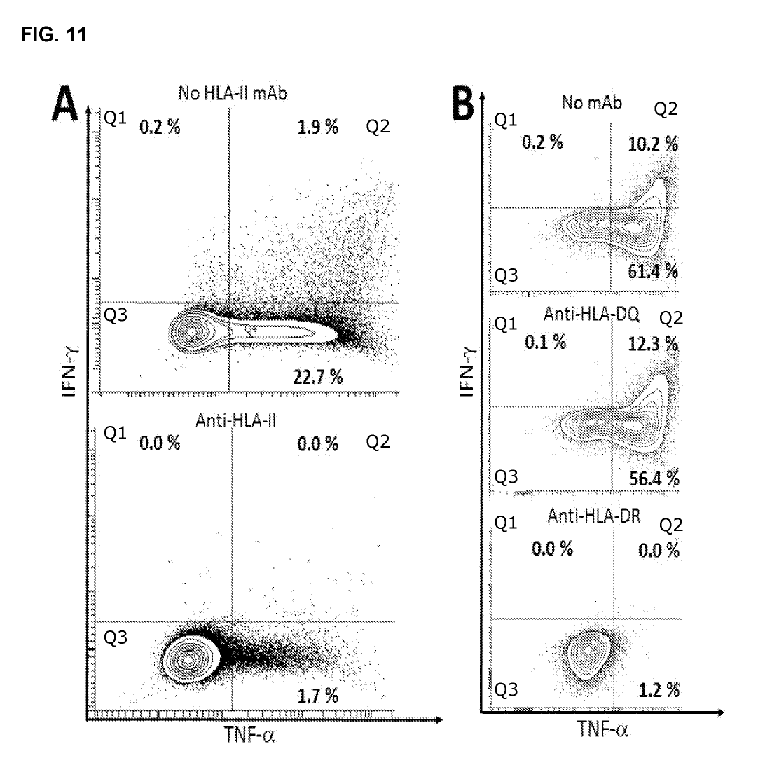

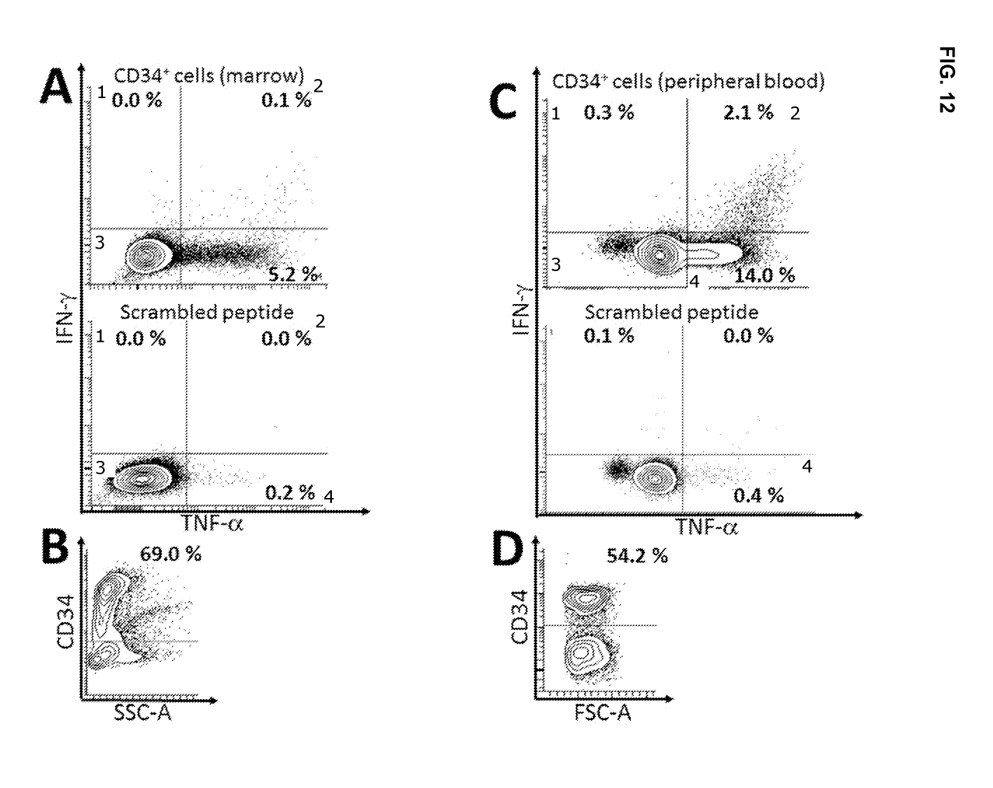

We next investigated whether the recognition of CALRmut target cells by CALRmut-specific T cells depended on CD4-HLA II interaction. We incubated CD14⁺ monocytes with an HLA II blocking monoclonal antibody (Tü39, BioLegend, San Diego, Calif., USA) for 30 min before using the cells. Blocking HLA II on the target cells significantly reduced the proportion of activated T-cells (FIG. 6A). Next, we treated the CALRLong1 specific T-cells with either an HLA-DQ blocking antibody or and HLA-DR blocking antibody (Abcam, Cambridge, UK) for 20 min, and hereafter the cells were stimulated with CALRLong1 peptide. T-cells that were treated with the HLA-DQ antibody (FIG. 11B, middle) were activated just as much as cells that were not treated with an antibody (FIG. 11B, top). However, no reaction was seen in T-cells that had been treated with the HLA-DR specific antibody (FIG. 11B, bottom).

The CD4⁺ CALRLong1-Specific T-Cells Recognize and are Activated by Stimulation with Autologous CD34⁺ Cells

The CALR exon 9 mutations are known to be an early mutational event, and thus CD34⁺ hematopoietic stem cells have been shown to harbor the mutation as well. Thus, we set out to investigate if the CD4⁺ CALRLong1-specific T-cells were able to recognize autologous CD34⁺ cells. We enriched CD34⁺ cells from both freshly aspirated bone marrow and from cryopreserved CD14⁺ depleted PBMC as described above, and used the CD34⁺ cells as target cells. Due to the limited number of target cells, the experiment with the marrow derived CD34⁺ cells was performed in duplicates at an effector:target ratio of 5:1. The experiment with the PBMC derived CD34⁺ cells was performed after the cells had rested for 48 h after enrichment in X-VIVO with 5% human serum. The marrow derived CD34⁺ cells were indeed able to activate the CD4⁺ CALRLong1-specific T-cells at an effector:target ratio of 3:1 (FIG. 12A, top) compared to stimulation with negative control peptide (FIG. 12A, bottom). Stimulation of the T-cells with the PBMC-derived CD34⁺ cells at an effector target ratio of 5:1 showed an even greater amount of activated T-cells (FIG. 12C, top) compared to T-cells stimulated with negative control peptide (FIG. 12C, bottom). The purity of the enriched target cells were >50% as analysed by flow cytometric analysis (FIGS. 12B and 12D).

The CD4⁺ CALRLong1-Specific T-Cells are Cytotoxic to Target Cells Displaying the CALRLong1 Epitope

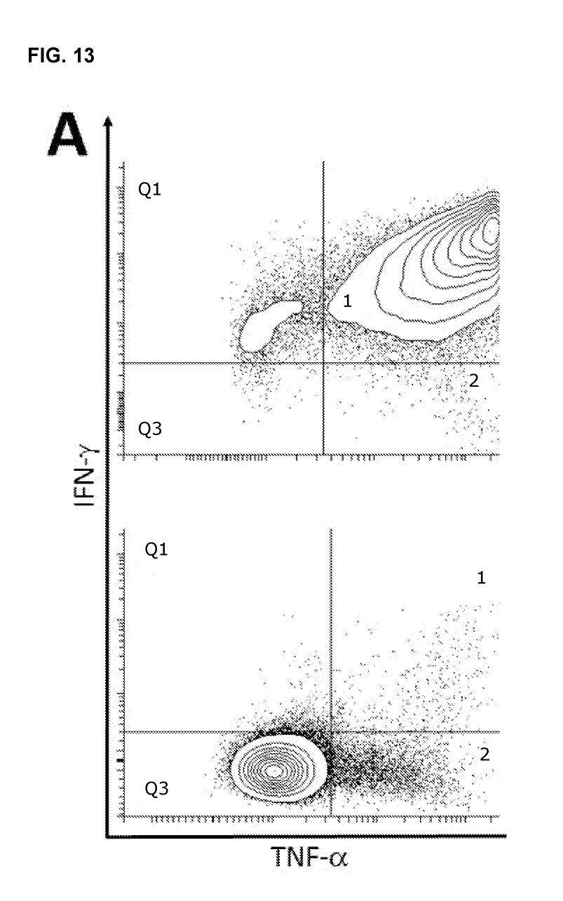

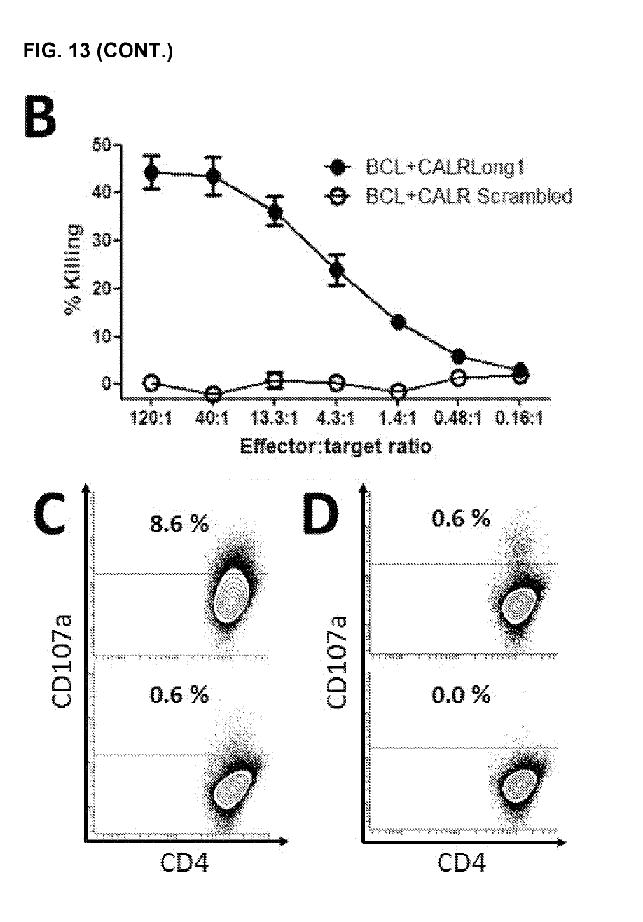

We pulsed autologous B-cells with CALRLong1 peptide or a scrambled control peptide. The CALRLong1-pulsed B-cells were widely recognized by the specific T cells (FIG. 13A). Next, we examined if CD4⁺ CALRLong1-specific T-cells would be cytotoxic to the target cells. In a standard Cr⁵¹-cytotoxicity assay, the CD4⁺ T-cells were indeed shown to be cytotoxic to CALRLong1 pulsed B-cells with a maximum killing effect of 45% at an effector:target ratio of 40:1 (FIG. 13B). Further, we investigated the expression of the degranulation marker CD107a on the CD4⁺ CALRLong1-specific T-cells after stimulation with B-cells pulsed with CALRLong1 or scrambled control peptide, and we showed that CD107a was upregulated on the T-cells that were stimulated with CALRLong1 peptide (FIG. 13C). Concurrently, we showed that a minor fraction of the CD4⁺ T-cells in the CALRLong1-specific bulk culture did upregulate CD107a upon stimulation with CALRLong1 peptide (FIG. 13D) demonstrating that also CD4⁺ T-cells in the bulk culture have cytotoxic capabilities.

### Example 3

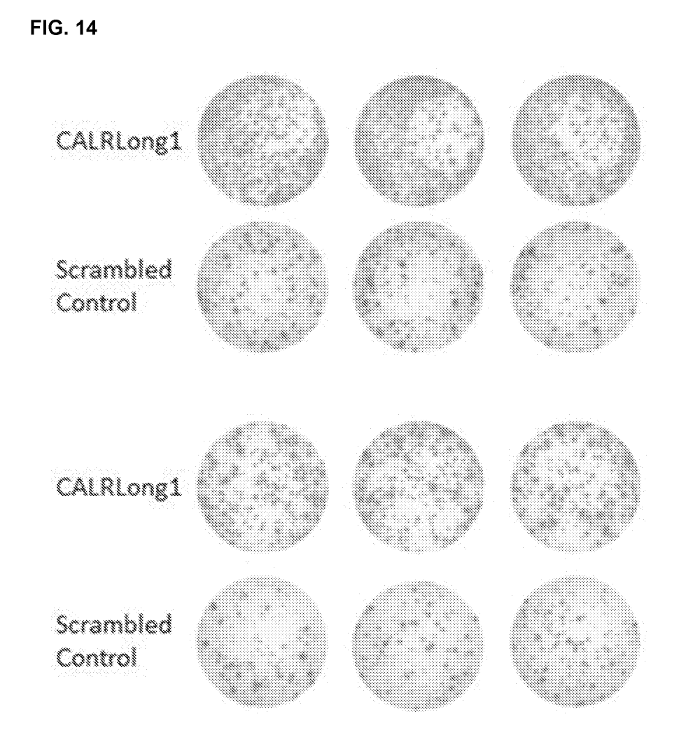

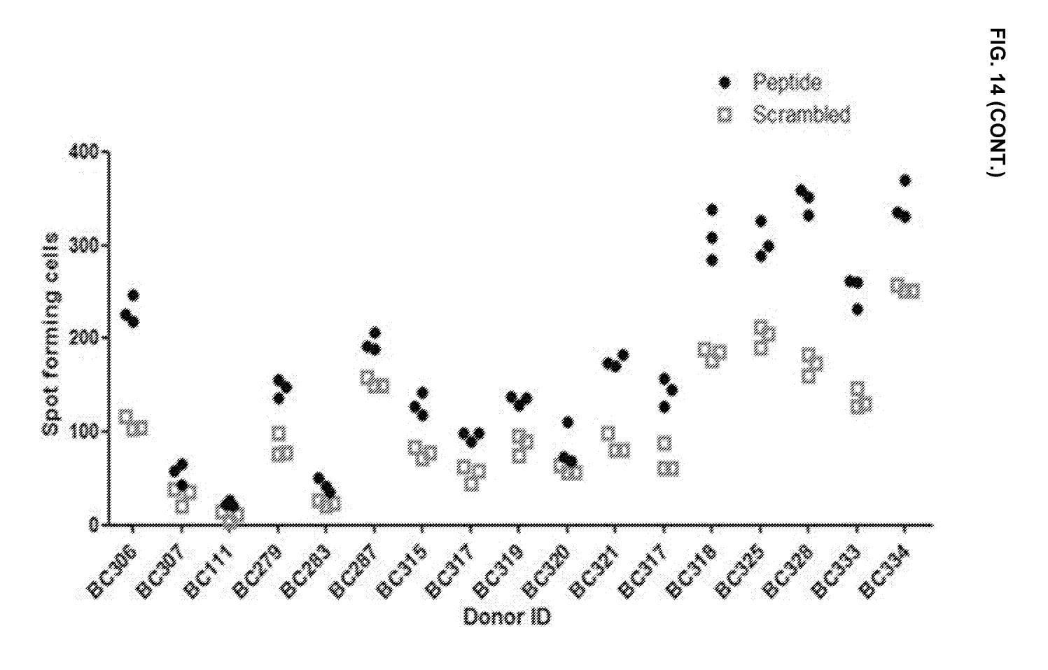

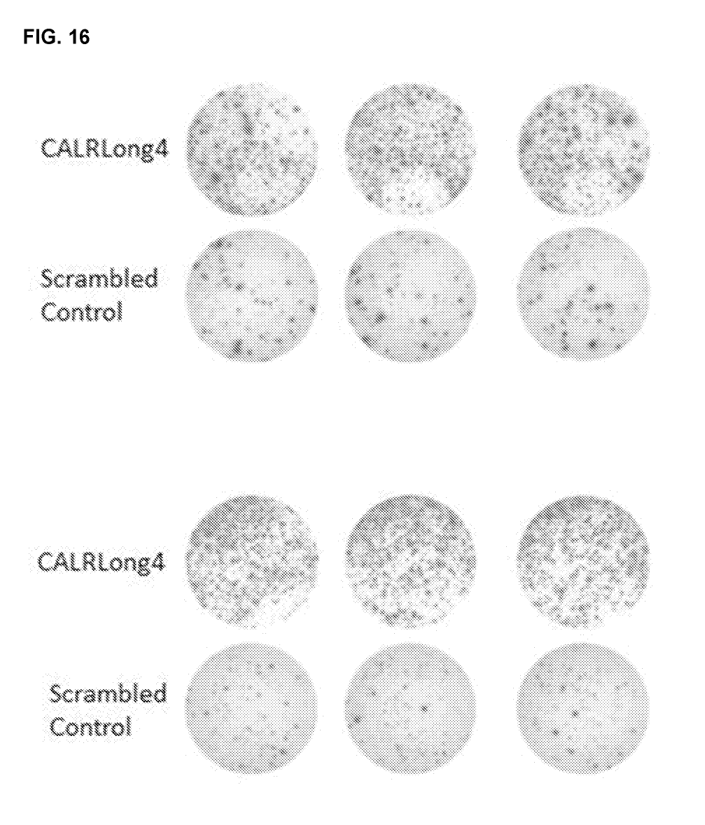

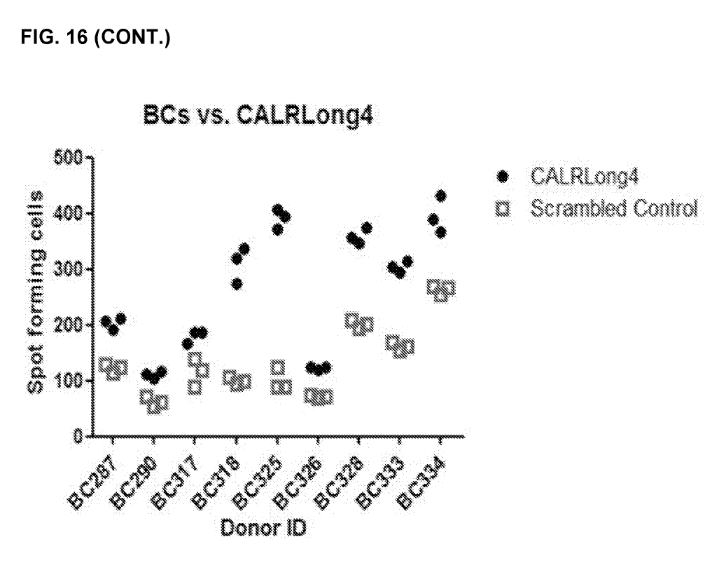

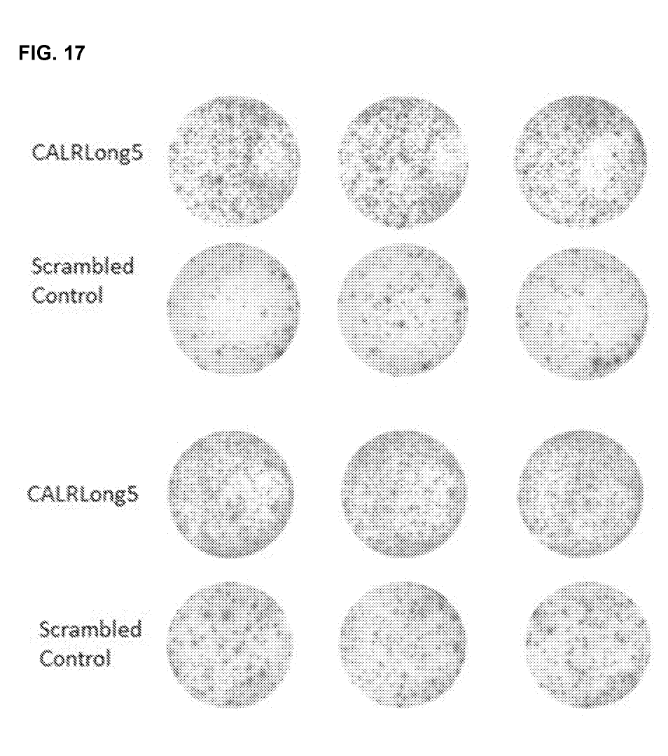

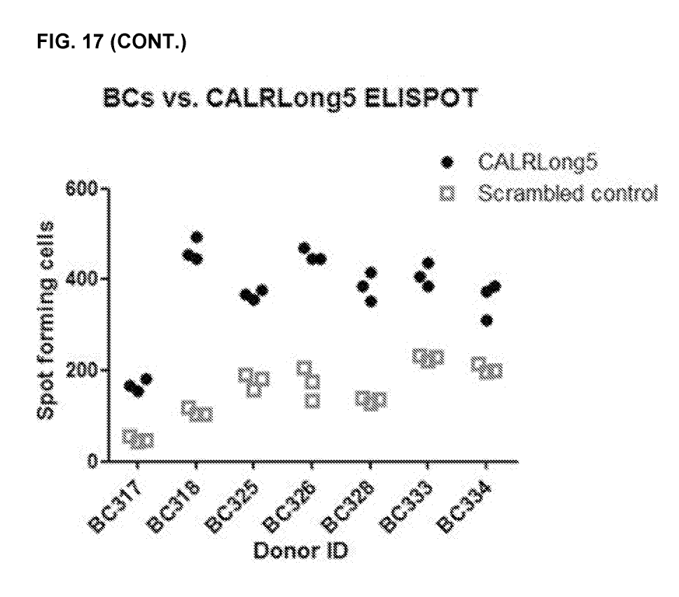

PBMC from healthy donors were analyzed by IFN-γ ELISPOTs as described in Example 1 for immune responses against the CARL-mutant epitopes CALRLong1 (RRMMRTKMRMRRMRRTRRKMRRKMSPARP), CALRLong2 (TRRKMRRKMSPARPRTSCREACLQGWTEA), CALRLong4 (RRMRRTRRKMRRKMSPARPRTSCREACLQGWTEA) and CALRLong5 (RRMMRTKMRMRRMRRTRRKMRRKMSPARPRTSCREACLQGWTEA). The healthy donors displayed strong immune responses against all these CALR-mutant epitopes (FIGS. 14, 15, 16, 17).

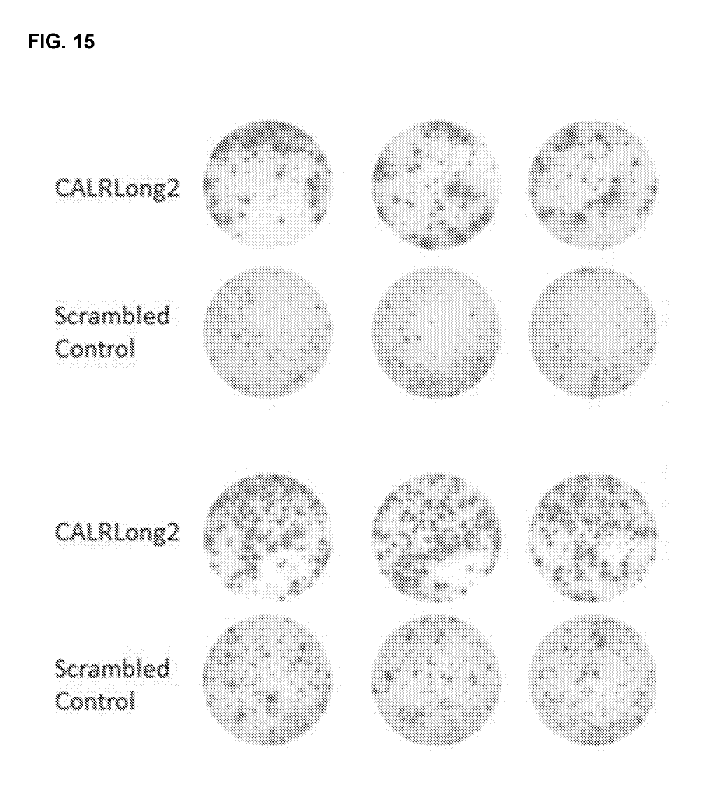

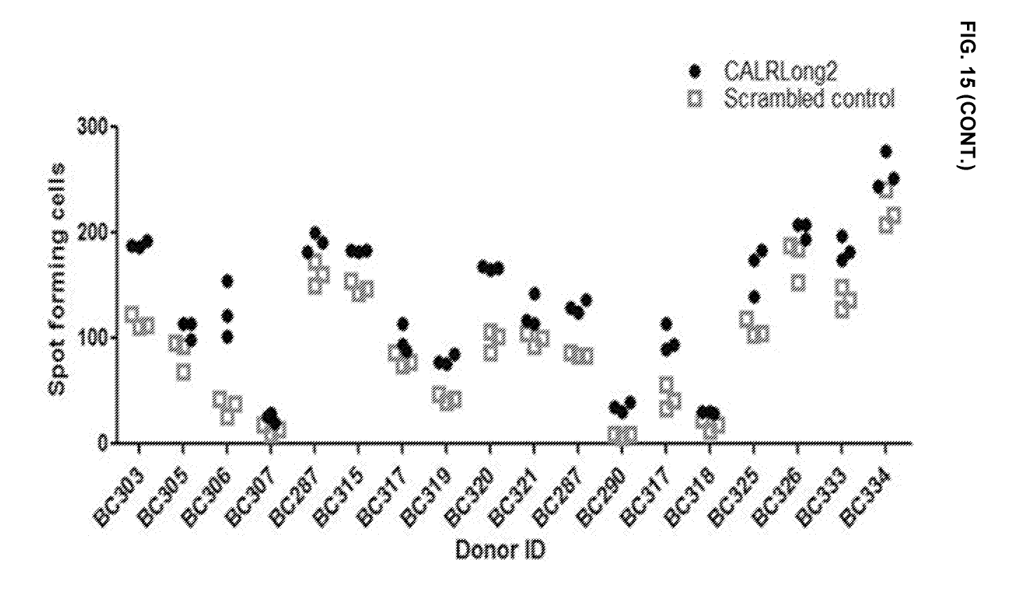

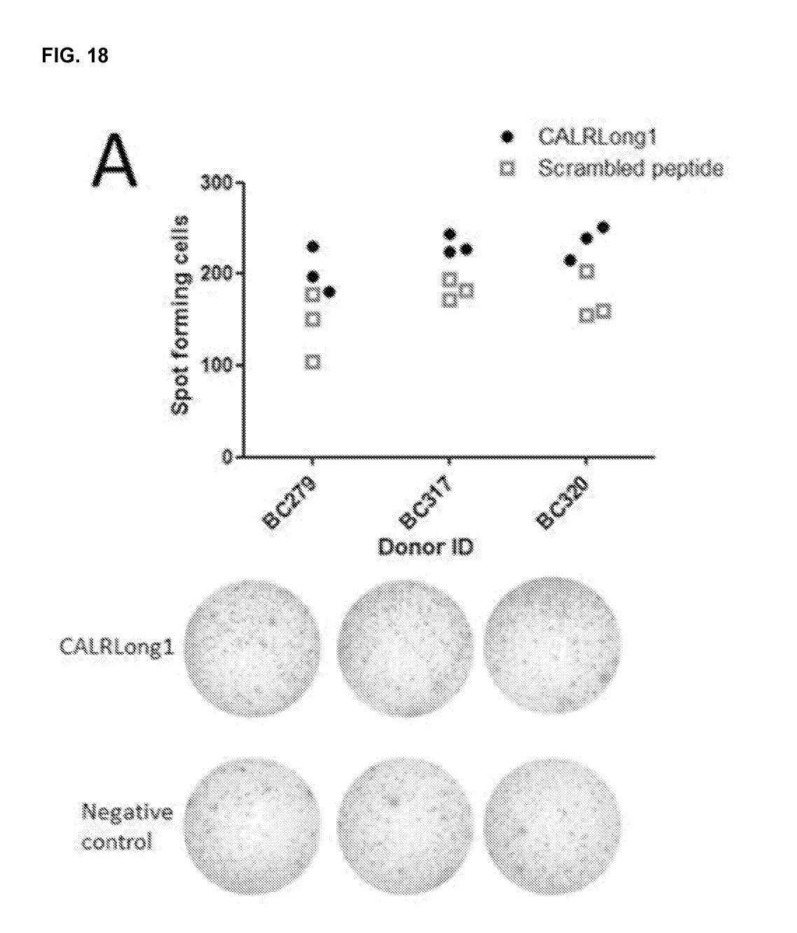

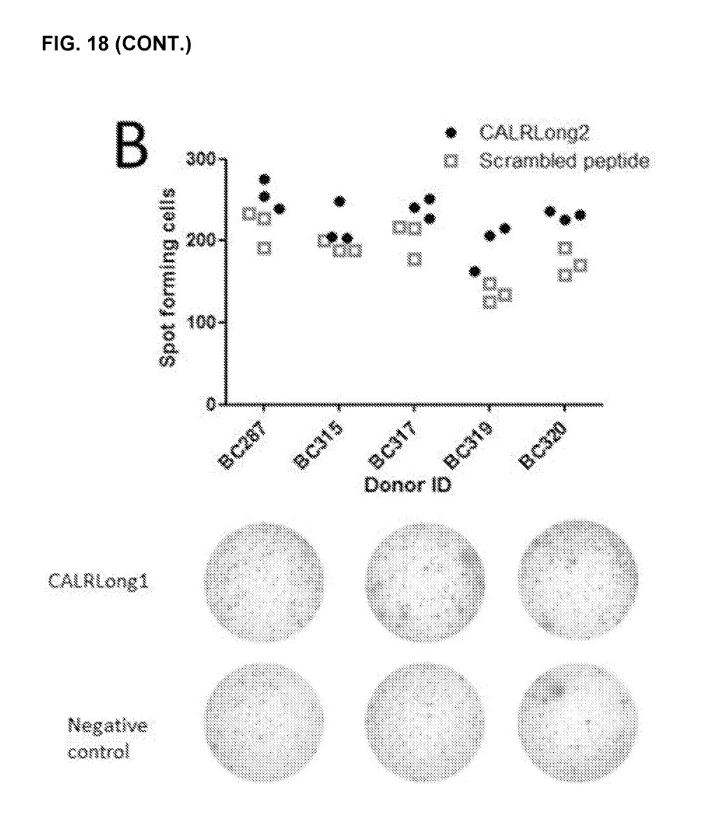

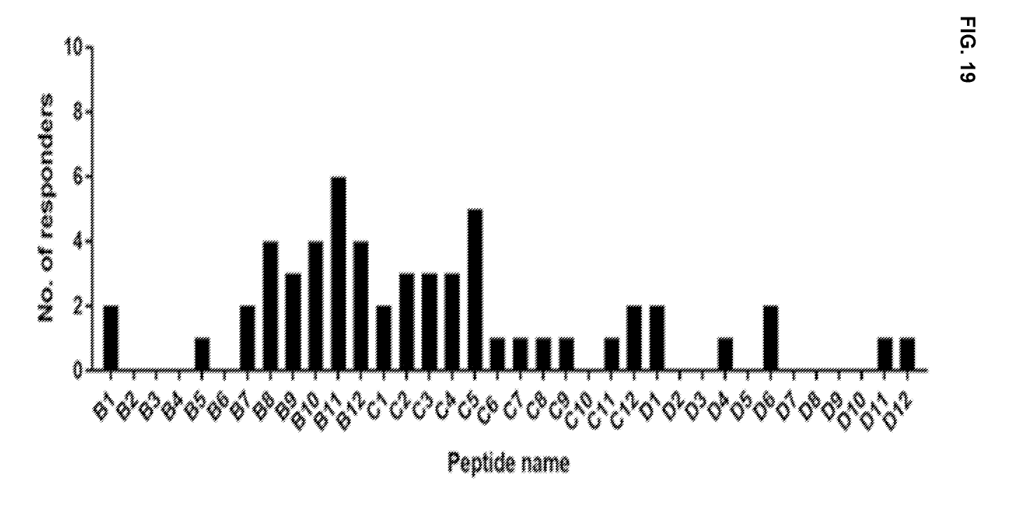

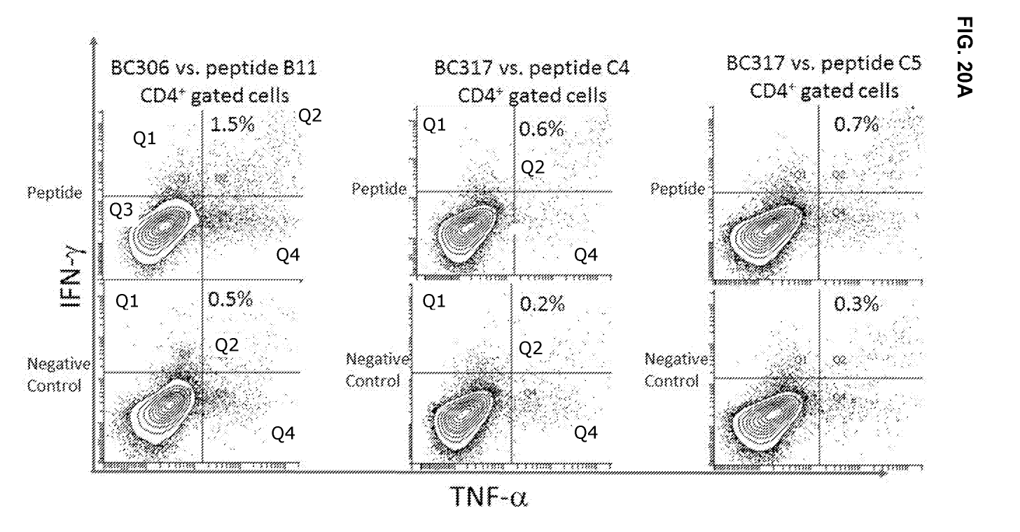

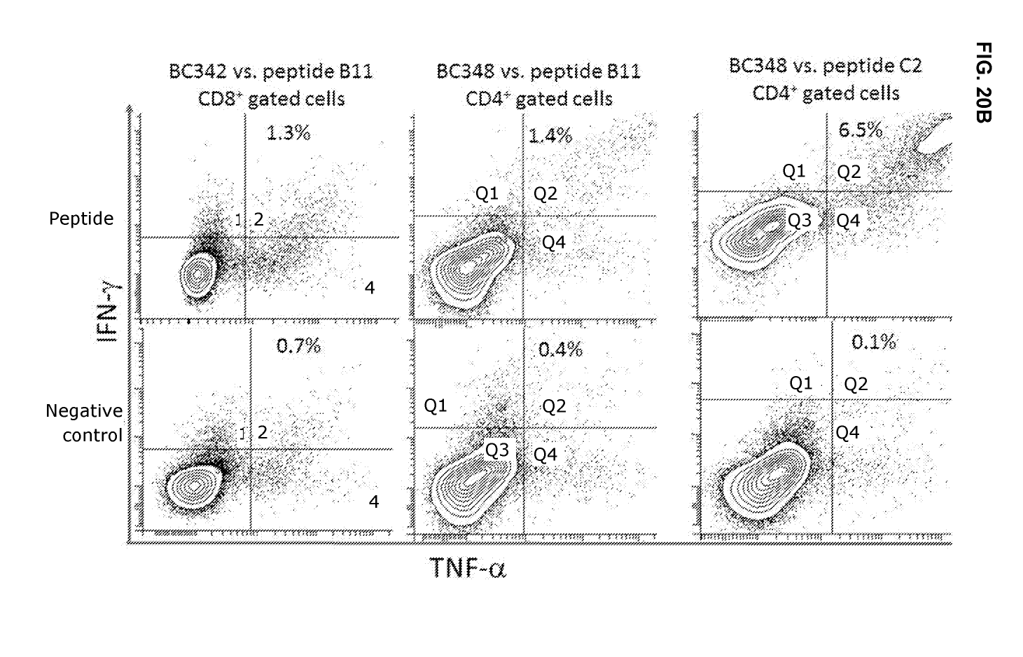

Healthy donors were also analyzed for reactivity against CALRLong1 (FIG. 18A) and CALRLong2 (FIG. 18B) using TNF-α ELISPOTs, and several of the donors displayed responses against the peptide. Of note, all patients with a TNF-α response against CALRLong1 and CALRLong2 did also display an IFN-g response in ELISPOT (FIGS. 14-15). When analyzing cells from healthy donors using intracellular cytokine stain we identified CD4+ T-cell responses in several donors against CALRLong1 peptide (not shown).

After these experiments we concluded that the entire CALR-mutant C-terminus was indeed immunogenic. However, we speculated that there might be an part of the mutant sequence which is more immunogenic than the rest of the sequence. We searched for this “immunogenic hotspot” by dividing the entire 44 amino acid sequence into 36 nonamer epitopes. Using IFN-γ ELISPOT we investigated the spontaneous immune response against these 36 nonamer epitopes in 10 healthy donors. All healthy donors displayed at least one immune response against one of the CALR-mutant nonamer epitopes, but it was apparent that most of the responses against the CALR-mutant epitope were confined to the first half of the CALR-mutant C-terminal (FIG. 19). To elucidate the phenotype of the IFN-g releasing cells we analyzed five donors with a strong response against some of the nonamer epitopes and once again, most of the responses were CD4+ T-cell responses (FIG. 20). However, one donor showed a CD8+ T-cell response against peptide B11 (FIG. 20).

### Example 4—JAK2

T-cells reactive against the mutant JAK2 peptide VLNYGVCFC (SEQ ID NO: 7) were prepared. The establishment of specific T cell cultures against a peptide was described previously (Munir S, Andersen G H, Met Ö, et al. HLA-restricted CTL that are specific for the immune checkpoint ligand PD-L1 occur with high frequency in cancer patients. Cancer Res. 2013). In short, peripheral blood mononuclear cells (PBMCs) from an HLA-A2 positive healthy donor were stimulated with JAK201-loaded autologous dendritic cells followed by stimulation with IL-2, IL-7, and IL-12. Generation of dendritic cells followed our previously published methods. (Munir S, Andersen G H, Met Ö, et al. HLA-restricted CTL that are specific for the immune checkpoint ligand PD-L1 occur with high frequency in cancer patients. Cancer Res. 2013). The HLA-A2 high affinity-binding epitope HIV-1 (ILKEPVHGV; SEQ ID NO: 26) and JAK201 (JAK201 wt) wild-type epitope (VLNYGVCVC; SEQ ID NO: 25) were used as controls. These cells were activated in response to the mutant JAK2 peptide and were capable of killing cells presenting the mutant JAK2 peptide. The T-cells were also activated when presented with cancer cells carrying the JAK2 mutation, and were capable of killing these cancer cells. This was dependent on the cells' ability to express the mutated JAK2 peptide, as shown by experiments performed with siRNA (FIG. 6).

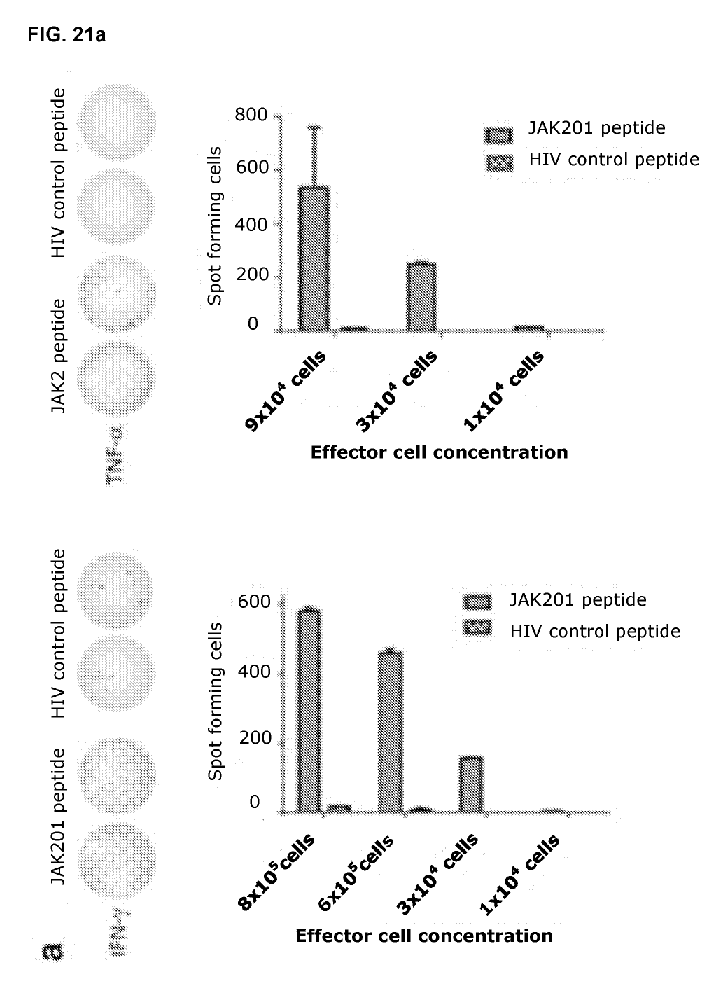

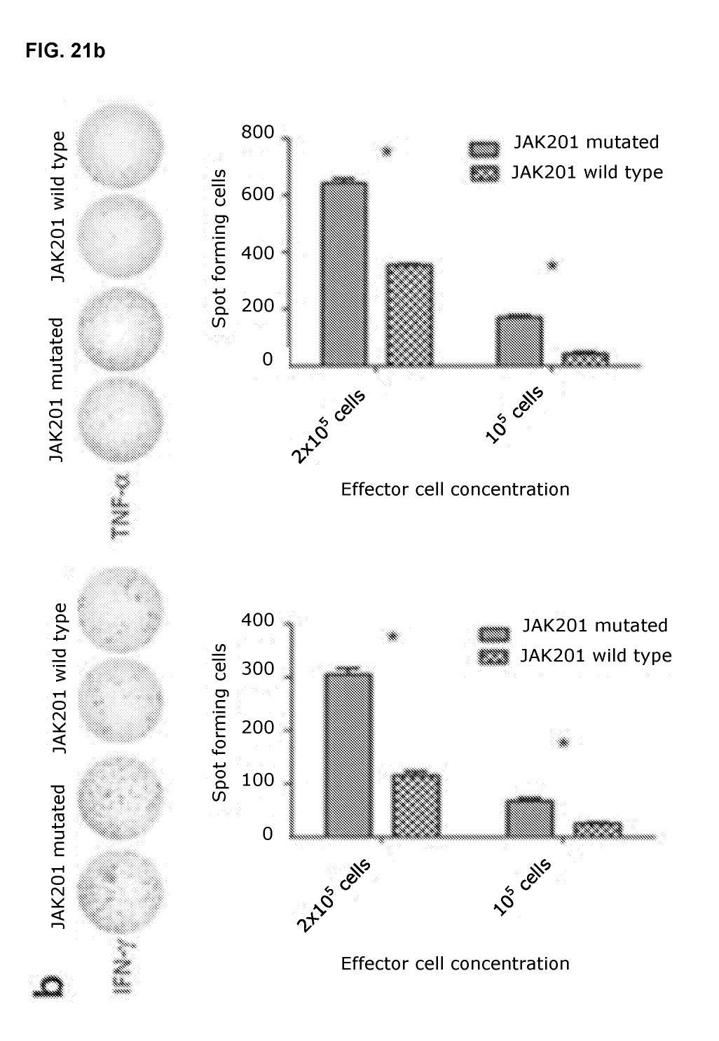

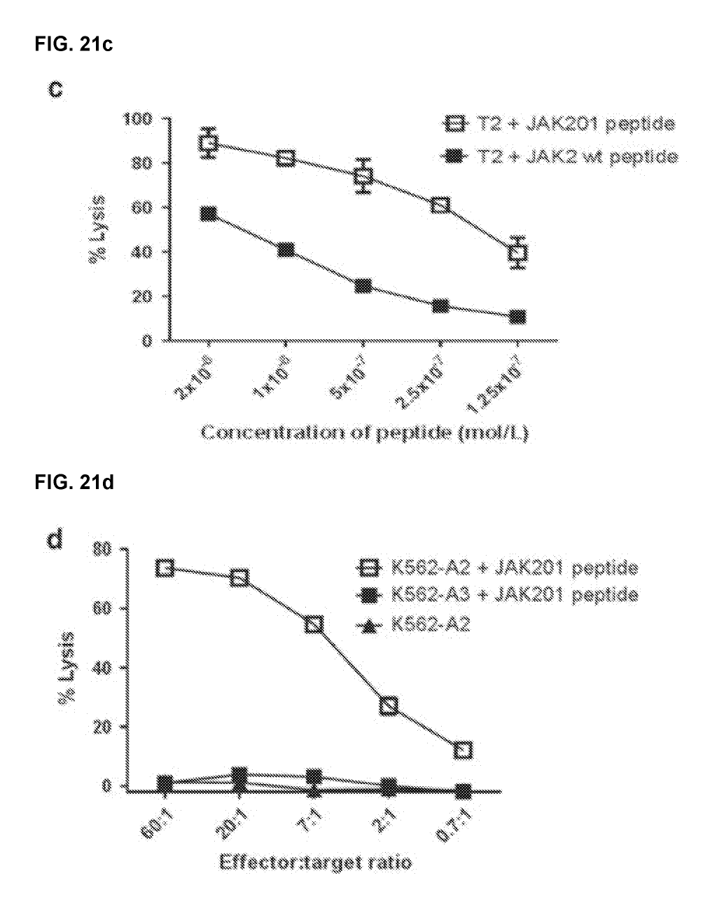

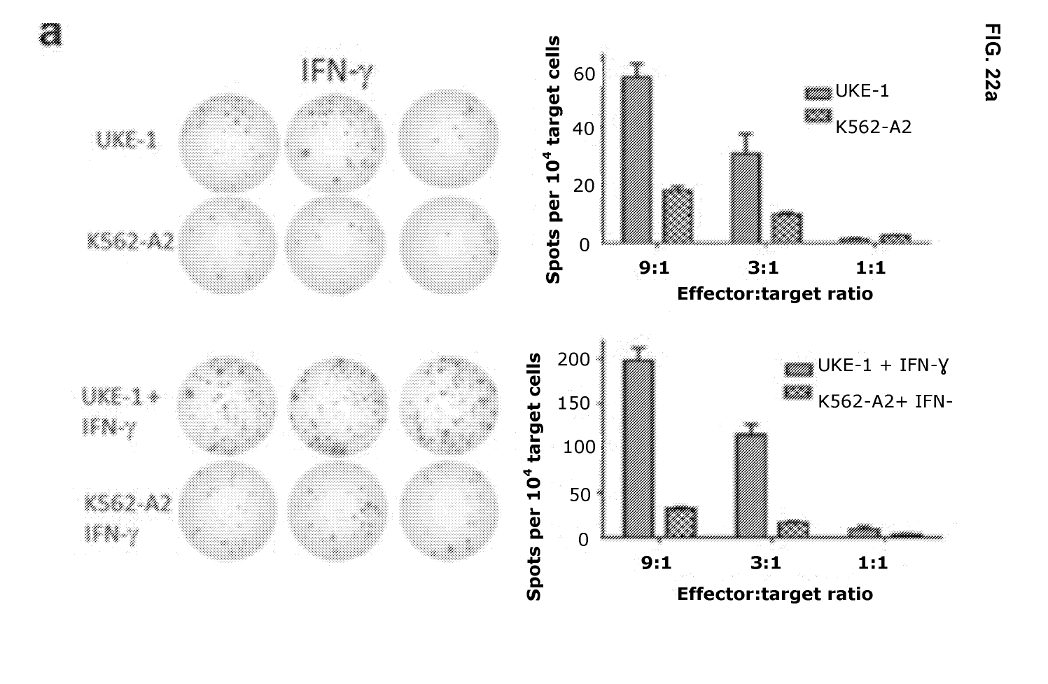

The specificity of the T cells was first analysed by ELISPOT. A significant JAK201 peptide-specific release of both IFN-γ and TNF-α was readily detectable by ELISPOT (FIG. 21a), suggesting that the T cells indeed were specific to the JAK201 peptide. The only difference between the JAK201 mutant peptide (SEQ ID NO: 7) and JAK201 wild-type (SEQ ID NO: 25) peptides is the valine-to-phenylalanine substitution, and, subsequently, we scrutinized the possible cross-reactivity between the two peptides. With ELISPOTs we showed that the specific T cells released significantly more IFN-γ as well as TNF-α when stimulated with JAK201 peptide compared with JAK201 wt peptide (FIG. 21b). This was confirmed by ICS (not shown). Using a Cr⁵¹ cytotoxicity assay, we observed some cross-reactivity between mutant and wild-type peptide. However, the specific T cells required a higher concentration of JAK201 wt peptide compared with JAK201 peptide to become activated to kill target cells, demonstrating that the T cells indeed had a higher affinity for the JAK201 peptide compared with JAK201 wt peptide (FIG. 21c).

Next, using standard Cr⁵¹ cytotoxicity assays, we showed that the CD8+ JAK201-specific T cells were indeed cytotoxic effector cells. Hence, TAP-deficient T2 cells were pulsed with JAK201 or HIV control peptide, and only cells displaying JAK201 peptide on the surface were killed by the T cells (data not shown). We further demonstrated that killing of the target cells was dependent on JAK201 presentation by HLA-A2 as the specific T cells were able to recognize only HLA-A2-transfected K562 cells pulsed with JAK201 peptide, whereas HLA-A2-transfected K562 cells without peptide or HLA-A3-transfected K562 cells pulsed with JAK201 peptide were not recognized (FIG. 21d).

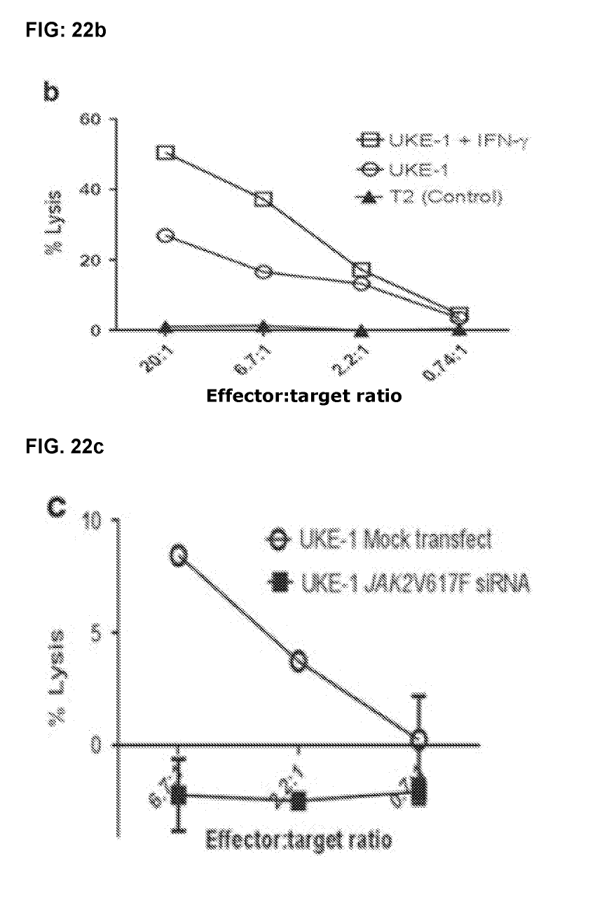

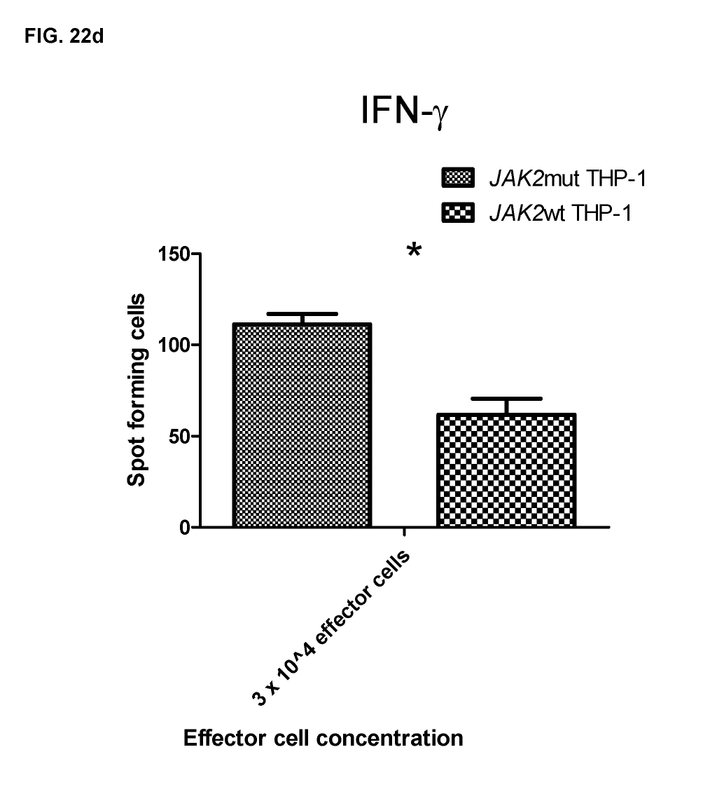

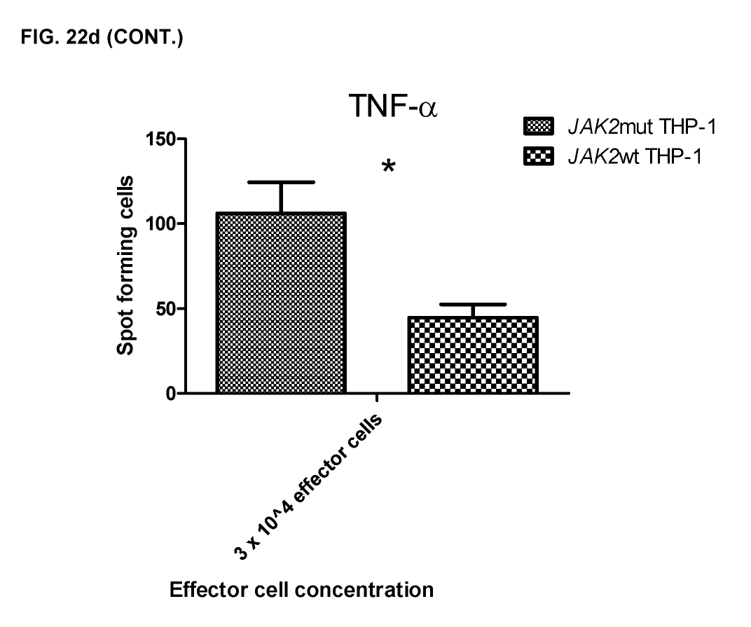

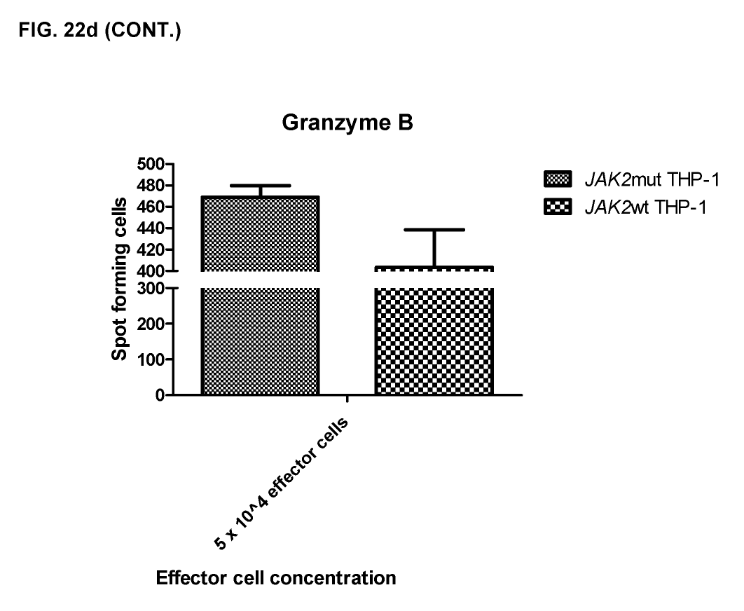

Next, we examined the capacity of JAK201-specific T cells to recognize and kill cancer cells harboring the JAK2V617F mutation. UKE-1, SET-2 and THP-1 are acute myeloid leukemia cancer cell lines that are HLA-A2-positive. THP-1 cells are JAK2 wt, whereas UKE-1 cells are homozygous and SET-2 are heterozygous for the JAK2V617F mutation. We first examined JAK201-specific T-cells reaction towards the UKE-1 cancer cell line. UKE-1 cells were able to activate the JAK201-specific T cells as stimulation of the T cells with UKE-1 cells resulted in the release of IFN-γ as detected by ELISPOT (FIG. 22a). Furthermore, stimulation of the JAK201-specific T cells with UKE-1 cells that had been pretreated with 100 U/ml IFN-γ 2 days before assaying resulted in an even greater release of IFN-γ from the T cells (FIG. 22a). IFN-γ is known to induce the immunoproteasome and to upregulate HLA class I on the cell surface overall enhancing the antigen presentation by the target cell. Next, UKE-1 cells were used as target cells in cytotoxicity assays, UKE-1 cells were efficiently lysed by JAK201-specific T cells, and lysis was augmented after treatment of the UKE-1 cells with IFN-γ (FIG. 22b). Initially, SET-2 cells were not lysed by the T cells, but after stimulation with IFN-γ the cancer cells were likewise lysed by the T cells (not shown).

Then we examined the importance of the intracellular expression of the JAK2V617F mutation for the recognition of target cells by JAK201-specific T cells. Thus to silence the JAK2V617F mutation, we transfected UKE-1 cells with JAK2V617F siRNA and used the transfected cells as target cells, and compared the recognition with mock transfected UKE-1 cells as previously described. Silencer siRNA duplex for targeted silencing of JAK2V617F with 3′-end overhang dT bases in the antisense strand (sense sequence 5′-GAGUAUGUUUCUGUGGAGATT-3′ (SEQ ID NO: 21), antisense sequence 5′-UCUCCACAGAAACAUACUCTT-3′ (SEQ ID NO: 22) for duplex 1; and sense sequence 5′-GGAGUAUGUUUCUGUGGAGTT-3′ (SEQ ID NO: 23), antisense sequence 5′-CUCCACAGAAACAUACUCCTT-3′ (SEQ ID NO: 24) for duplex 2) were obtained from Invitrogen (Invitrogen, Paisley, UK). For JAK2V617F silencing experiments, UKE-1 cells were transfected with JAK2V617F siRNA using electroporation parameters as described previously. Immediately after electroporation, UKE-1 cells were transferred to prewarmed RPMI 1640 containing 10% fetal calf serum, put in incubator and used as target cells in a standard Cr⁵¹ cytotoxicity assay 22 h after transfection. Importantly, killing of the siRNA-transfected UKE-1 cells was abrogated, whereas the mock-transfected cells were still killed by the JAK201-specific T cells (FIG. 22c). This showed that killing of target cells is indeed dependent on the intracellular expression of the JAK2V617F mutation. As an additional control we observed that the JAK201-specific T cells did not kill the JAK2 wt acute myeloid leukemia cancer cell line THP-1 in standard Cr⁵¹ cytotoxicity assays (data not shown).

Finally to confirm the recognition of cells expressing the JAK2V617F mutation, we transfected JAK2 wt THP-1 cells with mRNA encoding the JAK2V617F mutation using neural growth factor receptor (NGFR) encoding mRNA as control and used these cells as target cells in cytokine release and Granzyme B assays. The expression of NGFR in transfected cells was controlled at 20 h after transfection with PE-conjugated anti-NGFR mAb and showed that 74% of the cells were transfected. Using the transfected cells as target cells in ELISPOT assays, we were able to demonstrate that the JAK201-specific T cells specifically released IFN-γ, TNF-α and Granzyme B in response to JAK2V617F-transfected cells, compared with NGFR-transfected cells (FIG. 22d). More than 50% of patients with Philadelphia chromosomenegative chronic MPN harbor the JAK2V617F mutation. This acquired somatic mutation is found exclusively in myeloid malignancies, rendering it a cancer-specific antigen. JAK2V617F is consequently an attractive target for cancer immune therapy.

In the present study, we investigated whether JAK2V617F is specifically recognized by T cells. First, we established a CD8+ T-cell culture specific to an HLA-A2-restricted JAK2 peptide spanning the V617F mutation. These specific T cells released both TNF-α and IFN-γ when stimulated with the mutated JAK2 peptide and when stimulated with cancer cells harboring the JAK2V617F mutation. We further showed that the specific T cells selectively killed cancer cells that harbor the JAK2V617F mutation, and that treatment of the JAK2V617F-mutated cancer cells with IFN-γ augmented killing. Importantly, downregulation of the JAK2V617F mutation by transfection with JAK2V617F short interfering RNA abrogated T cell-mediated killing. Finally, the specific recognition of cells expressing the JAK2V617F mutation was confirmed, since knock in of the JAK2V617F mutation in JAK2 wt leukemia cells stimulated the JAK201-specific T cells to secrete more cytokine and Granzyme B.

In conclusion, the immune system is able to effectively target cancer cells carrying the JAK2V617F mutation, laying a foundation for specific cancer immune therapy as a new treatment modality for JAK2V617F. Hence, vaccination with JAK201 peptide or adoptive cell therapy targeting the JAK2V617F mutation may be ways to target JAK2V617F-positive MPNs. T cells can be engineered to express modified T-cell receptors targeting mutated targets. However, the cross-reactivity between the mutant and wild-type JAK2 epitopes must be further considered before exploiting this option.

### REFERENCES

Andersen M H, Reker S, Kvistborg P, Becker J C, thor Straten P. Spontaneous immunity against Bcl-xL in cancer patients. J Immunol 2005; 175: 2709-14.
Cabagnols X, Defour J P, Ugo V, Ianotto J C, Mossuz P, Mondet J et al. Differential association of calreticulin type 1 and type 2 mutations with myelofibrosis and essential thrombocytemia: relevance for disease evolution. Leukemia 2015; 29: 249-252.
Cox, J. C., and Coulter, A. R. (1997). Adjuvants—a classification and review of their modes of action. Vaccine 15, 248-56
Dunn G P, Bruce A T, Ikeda H, Old L J, Schreiber R D. Cancer immunoediting: from immunosurveillance to tumor escape. Nat Immunol 2002; 3: 991-998.
Hasselbalch H C. Myelofibrosis with myeloid metaplasia: the advanced phase of an untreated disseminated hematological cancer. Time to change our therapeutic attitude with early upfront treatment? Leuk Res 2009; 33: 11-18.
Jeromin S, Kohlmann A, Meggendorfer M, Schindela S, Perglerova K, Nadarajah N et al. Next-generation deep-sequencing detects multiple clones of CALR mutations in patients with BCR-ABL1 negative MPN. Leukemia 2016; 30: 973-976. Klampfl T, Gisslinger H, Harutyunyan A S, Nivarthi H, Rumi E, Milosevic J D et al. Somatic mutations of calreticulin in myeloproliferative neoplasms. N Engl J Med 2013; 369: 2379-2390.
Met Ö, Balslev E, Flyger H, Svane I M. High immunogenic potential of p53 mRNA-transfected dendritic cells in patients with primary breast cancer. Breast Cancer Res Treat 2011; 125: 395-406.
Moodie Z, Price L, Gouttefangeas C, Mander a., Janetzki S, Lower M et al. Response definition criteria for ELISPOT assays revisited. Cancer Immunol Immunother 2010; 59: 1489-1501.
Munir S, Andersen G H, Met Ö, Donia M, Frøsig T M, Larsen S K et al. HLA-restricted CTL that are specific for the immune checkpoint ligand PD-L1 occur with high frequency in cancer patients. Cancer Res 2013; 73: 1764-1776.
Munir S, Andersen G H, Svane I M, Andersen M H. The immune checkpoint regulator PD-L1 is a specific target for naturally occurring CD4(+) T cells. Oncoimmunology 2013; 2: e23991.
Nangalia J, Massie C E, Baxter E J, Nice F L, Gundem G, Wedge D C et al. Somatic CALR mutations in myeloproliferative neoplasms with nonmutated JAK2. N Engl J Med 2013; 369: 2391-2405.
Schumacher T N, Schreiber R D. Neoantigens in cancer immunotherapy. Science 2015; 348: 158 69-74.
Tefferi et al. Revised response criteria for myelofibrosis: International Working Group-Myeloproliferative Neoplasms Research and Treatment (IWG-MRT) and European LeukemiaNet (ELN) consensus report. Blood. 2013, 122; (8): 1395-1398.
Tefferi A, Lasho T L, Finke C, Belachew A, Wassie E, Ketterling R P et al. Type 1 vs type 2 calreticulin mutations in primary myelofibrosis: differences in phenotype and prognostic impact. Leukemia 2014; 28: 1568-1570.
Tefferi A, Wassie E A, Guglielmelli P, Gangat N, Belachew A A, Lasho T L et al. Type 1 vs Type 2 calreticulin mutations in essential thrombocythemia: a collaborative study of 1027 patients. Am J Hematol 2014; 89: E121-E124.

1.-110. (canceled)

111. A pharmaceutical composition comprising:
(a) an immunogenically active polypeptide comprising 8 to 50 consecutive amino acid residues from (i) amino acids 361 to 411 of SEQ ID NO: 9 or 10 or (ii) amino acids 607 to 657 of SEQ ID NO: 6, and
(b) a pharmaceutically acceptable excipient, adjuvant, and/or a preservative.

112. The composition according to claim 111, wherein the immunologically active polypeptide comprises the amino acid sequence of SEQ ID NO: 1, SEQ ID NO: 2, SEQ ID NO: 3, SEQ ID NO: 15, SEQ ID NO: 16, or SEQ ID NO: 17, or a functional homologue thereof having at least 90% sequence identity to SEQ ID NO: 1, SEQ ID NO: 2, SEQ ID NO: 3, SEQ ID NO: 15, SEQ ID NO: 16, or SEQ ID NO: 17.

113. The composition according to claim 111, wherein the immunologically active polypeptide comprises the amino acid sequence of SEQ ID NO: 7, or a functional homologue thereof having at least 90% sequence identity to SEQ ID NO: 7.

114. The composition according to claim 111, which further comprises cells which specifically recognize an exon 9 mutant of calreticulin (CALR), cells which specifically recognize a JAK2V617F mutant, interleukins, IFN-γ, an antibiotic agent, and/or an anti-cancer agent.

115. The composition according to claim 114, wherein the anti-cancer agent is a chemotherapeutic agent selected from Actimide, Azacitidine, Azathioprine, Bleomycin, Carboplatin, Capecitabine, Cisplatin, Chlorambucil, Cyclophosphamide, Cytarabine, Daunorubicin, Docetaxel, Doxifluridine, Doxorubicin, Epirubicin, Etoposide, Fludarabine, Fluorouracil, Gemcitabine, Hydroxyurea, Idarubicin, Irinotecan, Lenalidomide, Leucovorin, Mechlorethamine, Melphalan, Mercaptopurine, Methotrexate, Mitoxantrone, Oxaliplatin, Paclitaxel, Pemetrexed, Revlimid, Temozolomide, Teniposide, Thioguanine, Valrubicin, Vinblastine, Vincristine, Vindesine and Vinorelbine; and/or the antibiotic agent is selected from amoxicillin, penicillin, acyclovir and vidarabine.

116. The composition according to claim 111, further comprising an MHC Class I or Class II-restricted peptide.

117. The composition according to claim 111, further comprising an immunogenically active protein or peptide that is not CALR, an exon 9 mutant of CALR, JAK2, or a JAK2V617F mutant.

118. The composition according to claim 111, wherein the adjuvant is selected from bacterial DNA based adjuvants, oil/surfactant based adjuvants, viral dsRNA based adjuvants, Montanide ISA adjuvant, GM-CSF, and imidazochinilines.

119. The composition according to claim 111, wherein the composition comprises antigen presenting cells comprising the immunogenically active peptide fragment.

120. The composition of claim 119, wherein the antigen presenting cells are dendritic cells.

121. The composition according to claim 111, wherein the immunologically active polypeptide is in an amount effective for the treatment of a myeloproliferative disorder in a human in need thereof, wherein the myeloproliferative disorder is characterized by the expression of an exon 9 mutant of CALR comprising SEQ ID NO: 16 or SEQ ID NO: 1 and/or by the expression of a JAK2V617F mutant of SEQ ID NO: 6.

122. An isolated nucleic acid molecule comprising a nucleic acid sequence encoding an immunogenically active polypeptide comprising 8 to 50 consecutive amino acid residues from (i) amino acids 361 to 411 of SEQ ID NO: 9 or 10 or (ii) amino acids 607 to 657 of SEQ ID NO: 6.

123. A vector comprising the nucleic acid molecule of claim 122 and a nucleic acid sequence encoding a T cell stimulatory polypeptide.

124. A method of treating or preventing a clinical condition characterized by the expression of an exon 9 mutant of CALR and/or the expression of a JAK2V617F mutant, the method comprising administering to a human subject in need thereof an effective amount of the composition of claim 111.

125. The method of claim 124, wherein the clinical condition to be treated or prevented is a cancer or a myeloproliferative disorder.

126. The method of claim 125, wherein the myeloproliferative disorder is selected from essential thrombocythaemia, primary myelofibrosis, polycythemia vera, acute myeloid leukaemia, and chronic myeloid leukaemia.

127. The method of claim 124, further comprising one or more additional treatments selected from chemotherapy, radiotherapy, treatment with immunostimulating substances, gene therapy, treatment with antibodies, and treatment using dendritic cells.

128. The method according to claim 124, wherein the immunogenically active polypeptide is administered to the subject at a dose of from 50 μg to 500 μg.

129. A method of eliciting an immune response against a cancer cell or antigen presenting cell expressing an exon 9 mutant of CALR comprising the amino acid sequence of SEQ ID NO: 16 or SEQ ID NO: 1, which comprises administering the composition of claim 111 to an individual suffering from a clinical condition characterized by expression of the exon 9 mutant of CALR, wherein the immune response comprises one or more of:
(a) a cellular immune response in the individual;
(b) formation of cytotoxic T-cells which specifically recognize the exon 9 mutant of CALR; and/or
(c) production of T-cells having a cytotoxic effect against the cancer cell or antigen presenting cell expressing the exon 9 mutant of CALR.

130. A method of eliciting an immune response against a cancer cell or antigen presenting cell expressing a JAK2V617F mutant comprising the amino acid sequence of SEQ ID NO: 6, which method comprises administering the composition of claim 111 to an individual suffering from a clinical condition characterized by expression of the JAK2V617F mutant comprising the amino acid sequence of SEQ ID NO: 6, wherein the immune response comprises one or more of:
(a) eliciting a cellular immune response in the individual;
(b) eliciting the formation of cytotoxic T-cells which specifically recognize the JAK2V617F mutant; and/or
(c) production of T-cells having a cytotoxic effect against cancer cells expressing a mutant JAK2 protein comprising the amino acid sequence of SEQ ID NO: 7 and/or antigen presenting cells expressing a mutant JAK2 protein comprising the amino acid sequence of SEQ ID NO: 6.


SEQUENCE LISTING (26 sequences)

SEQ ID NO: 1 - protein, 34 aa, Homo sapiens
RRMRRTRRKMRRKMSPARPRTSCREACLQGWTEA

SEQ ID NO: 2 - protein, 29 aa, Homo sapiens
RRMMRTKMRMRRMRRTRRKMRRKMSPARP

SEQ ID NO: 3 - protein, 29 aa, Homo sapiens
TRRKMRRKMSPARPRTSCREACLQGWTEA

SEQ ID NO: 4 - protein, 57 aa, Homo sapiens
QDEEQRLKEEEEDKKRKEEEEAEDKEDDEDKDEDEEDEEDKEEDEEEDVPGQAKDEL

SEQ ID NO: 5 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 6 - protein, 1132 aa, Homo sapiens [sequence omitted]

SEQ ID NO: 7 - protein, 9 aa, Homo sapiens
VLNYGVCFC

SEQ ID NO: 8 - protein, 29 aa, Artificial sequence
  Scrambled peptide
  MISC_FEATURE (1)..(29): Scrmbled peptide used as control
MRRTMMMMMPRRRRRRKRRSKTRAPRMRK

SEQ ID NO: 9 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 10 - protein, 411 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRTRRMMRTKMRMRRMRRTRRKMRRKMSPARPRTSCREACLQGWTEA

SEQ ID NO: 11 - protein, 10 aa, Homo sapiens
RRMMRTKMRM

SEQ ID NO: 12 - protein, 45 aa, Homo sapiens
NCRRMMRTKMRMRRMRRTRRKMRRKMSPARPRTSCREACLQGWTE

SEQ ID NO: 13 - protein, 51 aa, Homo sapiens
QDEEQRTRRMMRTKMRMRRMRRTRRKMRRKMSPARPRTSCREACLQGWTEA

SEQ ID NO: 14 - protein, 43 aa, Homo sapiens
RRMMRTKMRMRRMRRTRRKMRRKMSPARPRTSCREACLQGWTE

SEQ ID NO: 15 - protein, 20 aa, Homo sapiens
KMRMRRMRRTRRKMRRKMSP

SEQ ID NO: 16 - protein, 36 aa, Homo sapiens
RMRRMRRTRRKMRRKMSPARPRTSCREACLQGWTEA

SEQ ID NO: 17 - protein, 44 aa, Homo sapiens
RRMMRTKMRMRRMRRTRRKMRRKMSPARPRTSCREACLQGWTEA

SEQ ID NO: 18 - protein, 19 aa, Artificial sequence
  Scrambled peptide 2
TSMMRRRRRRKRRKMMKRM

SEQ ID NO: 19 - DNA, 21 bp, Artificial sequence
  CALR siRNA sense sequence
ggagcaguuucuggacggatt

SEQ ID NO: 20 - DNA, 21 bp, Artificial sequence
  CALR siRNA antisense sequence
uccguccagaaacugcucctt

SEQ ID NO: 21 - DNA, 21 bp, Artificial sequence
  JAK2V617F duplex 1 sense sequence
gaguauguuucuguggagatt

SEQ ID NO: 22 - DNA, 21 bp, Artificial sequence
  JAK2V617F duplex 1 antisense sequence
ucuccacagaaacauacuctt

SEQ ID NO: 23 - DNA, 21 bp, Artificial sequence
  JAK2V617F duplex 2 sense sequence
ggaguauguuucuguggagtt

SEQ ID NO: 24 - DNA, 21 bp, Artificial sequence
  JAK2V617F duplex 2 antisense sequence
cuccacagaaacauacucctt

SEQ ID NO: 25 - protein, 9 aa, Homo sapiens
VLNYGVCVC

SEQ ID NO: 26 - protein, 9 aa, Homo sapiens
ILKEPVHGV